(19) United States
(12) Patent Application Publication
(10) Pub. No.: US 2018/0049457 A1
(43) Pub. Date: Feb. 22, 2018

(54) ENZYMATIC SYNTHESIS OF SOLUBLE GLUCAN FIBER

(71) Applicant: E I DU PONT DE NEMOURS AND COMPANY, Wilmington, DE (US)

(72) Inventors: Qiong Cheng, Wilmington, DE (US); Robert Dicosimo, Chadds Ford, PA (US); Arthur Ouwehand, Inga (FI); Zheng You, Wilmington, DE (US); Supaporn Suwannakham, Havertown, PA (US); Kevin D. Nagy, Wilmington, DE (US); Mark S. Payne, Wilmington, DE (US); Jahnavi Chandra Prasad, Wilmington, DE (US)

(21) Appl. No.: 15/313,238

(22) PCT Filed: May 22, 2015
(86) PCT No.: PCT/US15/32120

§ 371 Date: Nov. 22, 2016

Related U.S. Application Data
(60) Provisional application No. 62/004,290, filed on May 29, 2014.

(51) Int. Cl. A23L 29/269; C12P 19/08; C12P 19/18; A61K 8/73; A61K 31/716; A61K 8/36

(52) U.S. Cl. CPC A23L 29/269; C12P 19/08; C12P 19/18; A61K 8/73; C12Y 302/01059; A61K 8/36; C08L 2205/16; A61K 2800/10; C12Y 302/01011; A61K 31/716

(57) ABSTRACT

An enzymatically produced soluble α-glucan fiber composition is provided suitable for use as a digestion resistant fiber in food and feed applications. The soluble α-glucan fiber composition can be blended with one or more additional food ingredients to produce fiber-containing compositions. Methods for the production and use of compositions comprising the soluble α-glucan fiber are also provided.

### CROSS-REFERENCE TO RELATED APPLICATIONS

This application claims priority to and the benefit of U.S. provisional application No. 62/004,290, titled “Enzymatic Synthesis of Soluble Glucan Fiber,” filed May 29, 2014, the disclosure of which is incorporated by reference herein in its entirety.

### INCORPORATION BY REFERENCE OF THE SEQUENCE LISTING

The sequence listing provided in the file named “20150515_CL5833WOPCT_SequenceListing_ST25.txt” with a size of 1,026,328 bytes which was created on May 6, 2015 and which is filed herewith, is incorporated by reference herein in its entirety.

### FIELD OF THE INVENTION

This disclosure relates to a soluble α-glucan fiber, compositions comprising the soluble fiber, and methods of making and using the soluble α-glucan fiber. The soluble α-glucan fiber is highly resistant to digestion in the upper gastrointestinal tract, exhibits an acceptable rate of gas production in the lower gastrointestinal tract, is well tolerated as a dietary fiber, and has one or more beneficial properties typically associated with a soluble dietary fiber.

### BACKGROUND OF THE INVENTION

Dietary fiber (both soluble and insoluble) is a nutrient important for health, digestion, and preventing conditions such as heart disease, diabetes, obesity, diverticulitis, and constipation. However, most humans do not consume the daily recommended intake of dietary fiber. The 2010 Dietary Fiber Guidelines for Americans (U.S. Department of Agriculture and U.S. Department of Health and Human Services. Dietary Guidelines for Americans, 2010. 7th Edition, Washington, D.C.: U.S. Government Printing Office, December 2010) reports that the insufficiency of dietary fiber intake is a public health concern for both adults and children. As such, there remains a need to increase the amount of daily dietary fiber intake, especially soluble dietary fiber suitable for use in a variety of food applications.

Historically, dietary fiber was defined as the non-digestible carbohydrates and lignin that are intrinsic and intact in plants. This definition has been expanded to include carbohydrate polymers with three or more monomeric units that are not significantly hydrolyzed by the endogenous enzymes in the upper gastrointestinal tract of humans and which have a beneficial physiological effect demonstrated by generally accepted scientific evidence. Soluble oligosaccharide fiber products (such as oligomers of fructans, glucans, etc.) are currently used in a variety of food applications. However, many of the commercially available soluble fibers have undesirable properties such as low tolerance (causing undesirable effects such as abdominal bloating or gas, diarrhea, etc.), lack of digestion resistance, instability at low pH (e.g., pH 4 or less), high cost or a production process that requires at least one acid-catalyzed heat treatment step to randomly rearrange the more-digestible glycosidic bonds (for example, α-(1,4) linkages in glucans) into more highly-branched compounds with linkages that are more digestion-resistant. A process that uses only naturally occurring enzymes to synthesize suitable glucan fibers from a safe and readily-available substrate, such as sucrose, may be more attractive to consumers.

Various bacterial species have the ability to synthesize dextran oligomers from sucrose. Jeanes et al. (JACS (1954) 76:5041-5052) describe dextrans produced from 96 strains of bacteria. The dextrans were reported to contain a significant percentage (50-97%) of α-(1,6) glycosidic linkages with varying amounts of α-(1,3) and α-(1,4) glycosidic linkages. The enzymes present (both number and type) within the individual strains were not reported, and the dextran profiles in certain strains exhibited variability, where the dextrans produced by each bacterial species may be the product of more than one enzyme produced by each bacterial species.

Glucosyltransferases (glucansucrases; GTFs) belonging to glucoside hydrolase family 70 are able to polymerize the D-glucosyl units of sucrose to form homooligosaccharides or homopolysaccharides. Glucansucrases are further classified by the type of saccharide oligomer formed. For example, dextransucrases are those that produce saccharide oligomers with predominantly α-(1,6) glycosidic linkages (“dextrans”), and mutansucrases are those that tend to produce insoluble saccharide oligomers with a backbone rich in α-(1,3) glycosidic linkages. Mutansucrases are characterized by common amino acids. For example, A. Shimamura et al. (J. Bacteriology, (1994) 176:4845-4850) investigated the structure-function relationship of GTFs from Streptococcus mutans GS5, and identified several amino acid positions which influence the nature of the glucan product synthesized by GTFs where changes in the relative amounts of α-(1,3)- and α-(1,6)-anomeric linkages were produced. Reuteransucrases tend to produce saccharide oligomers rich in α-(1,4), α-(1,6), and α-(1,4,6) glycosidic linkages, and alternansucrases are those that tend to produce saccharide oligomers with a linear backbone comprised of alternating α-(1,3) and α-(1,6) glycosidic linkages. Some of these enzymes are capable of introducing other glycosidic linkages, often as branch points, to varying degrees. V. Monchois et al. (FEMS Microbiol Rev., (1999) 23:131-151) discusses the proposed mechanism of action and structure-function relationships for several glucansucrases. H. Leemhuis et al. (J. Biotechnol., (2013) 163:250-272) describe characteristic three-dimensional structures, reactions, mechanisms, and α-glucan analyses of glucansucrases.

A non-limiting list of patents and published patent applications describing the use of glucansucrases (wild type, truncated or variants thereof) to produce saccharide oligomers has been reported for dextran (U.S. Pat. Nos. 4,649,058 and 7,897,373; and U.S. Patent Appl. Pub. No. 2011-0178289A1), reuteran (U.S. Patent Application Publication No. 2009-0297663A1 and U.S. Pat. No. 6,867,026), alternan and/or maltoalternan oligomers (“MAOs”) (U.S. Pat. Nos. 7,402,420 and 7,524,645; U.S. Patent Appl. Pub. No. 2010-0122378A1; and European Patent EP1151085B1), α-(1,2) branched dextrans (U.S. Pat. No. 7,439,049), and a mixed-linkage saccharide oligomer (lacking an alternan-like backbone) comprising a mix of α-(1,3), α-(1,6), and α-(1,3,6) linkages (U.S. Patent Appl. Pub. No. 2005-0059633A1). U.S. Patent Appl. Pub. No. 2009-0300798A1 to Kol-Jakon et al. discloses genetically modified plant cells expressing a mutansucrase to produce modified starch.

Enzymatic production of isomaltose, isomaltooligosaccharides, and dextran using a combination of a glucosyltransferase and an α-glucanohydrolase has been reported. U.S. Pat. No. 2,776,925 describes a method for enzymatic production of dextran of intermediate molecular weight comprising the simultaneous action of dextransucrase and dextranase. U.S. Pat. No. 4,861,381A describes a method to enzymatically produce a composition comprising 39-80% isomaltose using a combination of a dextransucrase and a dextranase. Goulas et al. (Enz. Microb. Tech (2004) 35:327-338 describes batch synthesis of isomaltooligosaccharides (IMOs) from sucrose using a dextransucrase and a dextranase. U.S. Pat. No. 8,192,956 discloses a method to enzymatically produce isomaltooligosaccharides (IMOs) and low molecular weight dextran for clinical use using a recombinantly expressed hybrid gene comprising a gene encoding an α-glucanase and a gene encoding dextransucrase fused together; wherein the glucanase gene is a gene from Arthrobacter sp., wherein the dextransucrase gene is a gene from Leuconostoc sp.

Hayacibara et al. (Carb. Res. (2004) 339:2127-2137) describe the influence of mutanase and dextranase on the production and structure of glucans formed by glucosyltransferases from sucrose within dental plaque. The reported purpose of the study was to evaluate the production and the structure of glucans synthesized by GTFs in the presence of mutanase and dextranase, alone or in combination, in an attempt to elucidate some of the interactions that may occur during the formation of dental plaque.

Mutanases (glucan endo-1,3-α-glucanohydrolases) are produced by some fungi, including Trichoderma, Aspergillus, Penicillium, and Cladosporium, and by some bacteria, including Streptomyces, Flavobacterium, Bacteroides, Bacillus, and Paenibacillus. W. Suyotha et al., (Biosci, Biotechnol. Biochem., (2013) 77:639-647) describe the domain structure and impact of domain deletions on the activity of an α-1,3-glucanohydrolases from Bacillus circulans KA-304. Y. Hakamada et al. (Biochimie, (2008) 90:525-533) describe the domain structure analysis of several mutanases, and a phylogenetic tree for mutanases is presented. I. Shimotsuura et al, (Appl. Environ. Microbiol., (2008) 74:2759-2765) report the biochemical and molecular characterization of mutanase from Paenibacillus sp. Strain RM1, where the N-terminal domain had strong mutan-binding activity but no mutanase activity, whereas the C-terminal domain was responsible for mutanase activity but had mutan-binding activity significantly lower than that of the intact protein. C. C. Fuglsang et al. (J. Biol. Chem., (2000) 275:2009-2018) describe the biochemical analysis of recombinant fungal mutanases (endoglucanases), where the fungal mutanases are comprised of a NH₂-terminal catalytic domain and a putative COOH-terminal polysaccharide binding domain.

Dextranases (α-1,6-glucan-6-glucanohydrolases) are enzymes that hydrolyzes α-1,6-linkages of dextran. N. Suzuki et al. (J. Biol. Chem., (2012) 287: 19916-19926) describes the crystal structure of Streptococcus mutans dextranase and identifies three structural domains, including domain A that contains the enzyme's catalytic module, and a dextran-binding domain C; the catalytic mechanism was also described relative to the enzyme structure. A. M. Larsson et al. (Structure, (2003) 11:1111-1121) reports the crystal structure of dextranase from Penicillium minioluteum, where the structure is used to define the reaction mechanism. H-K Kang et al. (Yeast, (2005) 22:1239-1248) describes the characterization of a dextranase from Lipomyces starkeyi. T. Igarashi et al. (Microbiol. Immunol., (2004) 48:155-162) describe the molecular characterization of dextranase from Streptococcus rattus, where the conserved region of the amino acid sequence contained two functional domains, catalytic and dextran-binding sites.

Various saccharide oligomer compositions have been reported in the art. For example, U.S. Pat. No. 6,486,314 discloses an α-glucan comprising at least 20, up to about 100,000 α-anhydroglucose units, 38-48% of which are 4-linked anhydroglucose units, 17-28% are 6-linked anhydroglucose units, and 7-20% are 4,6-linked anhydroglucose units and/or gluco-oligosaccharides containing at least two 4-linked anhydroglucose units, at least one 6-linked anhydroglucose unit and at least one 4,6-linked anhydroglucose unit. U.S. Patent Appl. Pub. No. 2010-0284972A1 discloses a composition for improving the health of a subject comprising an α-(1,2)-branched α-(1,6) oligodextran. U.S. Patent Appl. Pub. No. 2011-0020496A1 discloses a branched dextrin having a structure wherein glucose or isomaltooligosaccharide is linked to a non-reducing terminal of a dextrin through an α-(1,6) glycosidic bond and having a DE of 10 to 52. U.S. Pat. No. 6,630,586 discloses a branched maltodextrin composition comprising 22-35% (1,6) glycosidic linkages; a reducing sugars content of <20%; a polymolecularity index (Mp/Mn) of <5; and number average molecular weight (Mn) of 4500 g/mol or less. U.S. Pat. No. 7,612,198 discloses soluble, highly branched glucose polymers, having a reducing sugar content of less than 1%, a level of α-(1,6) glycosidic bonds of between 13 and 17% and a molecular weight having a value of between 0.9×10⁵ and 1.5×10⁵ daltons, wherein the soluble highly branched glucose polymers have a branched chain length distribution profile of 70 to 85% of a degree of polymerization (DP) of less than 15, of 10 to 14% of DP of between 15 and 25 and of 8 to 13% of DP greater than 25.

Saccharide oligomers and/or carbohydrate compositions comprising the oligomers have been described as suitable for use as a source of soluble fiber in food applications (U.S. Pat. No. 8,057,840 and U.S. Patent Appl. Pub. Nos. 2010-0047432A1 and 2011-0081474A1). U.S. Patent Appl. Pub. No. 2012-0034366A1 discloses low sugar, fiber-containing carbohydrate compositions which are reported to be suitable for use as substitutes for traditional corn syrups, high fructose corn syrups, and other sweeteners in food products.

There remains a need to develop new soluble α-glucan fiber compositions that are digestion resistant, exhibit a relatively low level and/or slow rate of gas formation in the lower gastrointestinal tract, are well-tolerated, have low viscosity, and are suitable for use in foods and other applications. Preferably the α-glucan fiber compositions can be enzymatically produced from sucrose using enzymes already associated with safe use in humans.

### SUMMARY OF THE INVENTION

A soluble α-glucan fiber composition is provided that is suitable for use in a variety of applications including, but not limited to, food applications, compositions to improve gastrointestinal health, and personal care compositions. The soluble fiber composition may be directly used as an ingredient in food or may be incorporated into carbohydrate compositions suitable for use in food applications.

A process for producing the soluble α-glucan fiber composition is also provided.

Methods of using the soluble fiber composition or carbohydrate compositions comprising the soluble fiber composition in food applications are also provided. In certain aspects, methods are provided for improving the health of a subject comprising administering the present soluble fiber composition to a subject in an amount effective to exert at least one health benefit typically associated with soluble dietary fiber such as altering the caloric content of food, decreasing the glycemic index of food, altering fecal weight and supporting bowel function, altering cholesterol metabolism, provide energy-yielding metabolites through colonic fermentation, and possibly providing prebiotic effects.

A soluble α-glucan fiber composition is provided comprising, on a dry solids basis, the following:

a. at least 75% α-(1,3) glycosidic linkages;

b. less than 25% α-(1,6) glycosidic linkages;

c. less than 10% α-(1,3,6) glycosidic linkages;

d. a weight average molecular weight of less than 5000 Daltons;

e. a viscosity of less than 0.25 Pascal second (Pa·s) at 12 wt % in water 20° C.;

f. a dextrose equivalence (DE) in the range of 4 to 40; and

g. a digestibility of less than 12% as measured by the Association of Analytical Communities (AOAC) method 2009.01;

h. a solubility of at least 20% (w/w) in water at 25° C.; and

i. a polydispersity index of less than 5.

In another embodiment, a method to produce a soluble α-glucan fiber composition is provided, the method comprising:

a. providing a set of reaction components comprising:
i. sucrose;
ii. at least one glucosyltransferase capable of catalyzing the synthesis of glucan polymers having at least 75% α-(1,3) glycosidic linkages;
iii. at least one α-glucanohydrolase capable of hydrolyzing glucan polymers having one or more α-(1,3) glycosidic linkages or one or more α-(1,6) glycosidic linkages; and
iv. optionally one or more acceptors;

b. combining the set of reaction components under suitable aqueous reaction conditions whereby a product comprising a soluble α-glucan fiber composition is produced; and

c. optionally isolating the soluble α-glucan fiber composition from the product of step (b).

In another embodiment, a method to produce the soluble α-glucan fiber composition described above is provided, the method comprising:

a. providing a set of reaction components comprising:
i. sucrose;
ii. at least one glucosyltransferase capable of catalyzing the synthesis of glucan polymers having at least 75% α-(1,3) glycosidic linkages;
iii. at least one α-glucanohydrolase capable of hydrolyzing glucan polymers having one or more α-(1,3) glycosidic linkages or one or more α-(1,6) glycosidic linkages; and
iv. optionally one or more acceptors;

b. combining the set of reaction components under suitable aqueous reaction conditions to form a single reaction mixture, whereby a product mixture comprising glucose oligomers is formed;

c. isolating the soluble α-glucan fiber composition as described above from the product mixture comprising glucose oligomers; and

d. optionally concentrating the soluble α-glucan fiber composition.

In another embodiment, a method is provided to produce the soluble α-glucan fiber composition as described above, the method comprising:

a. providing a set of reaction components comprising:
i. sucrose;
ii. at least one glucosyltransferase capable of catalyzing the synthesis of glucan polymers having one or more α-(1,3) glycosidic linkages; and
iii. optionally one or more acceptors;

b. combining the set of reaction components under suitable aqueous reaction conditions to form a single reaction mixture, wherein the reaction conditions comprise a reaction temperature greater than 45° C. and less than 55° C., whereby a product mixture comprising glucose oligomers is formed;

c. isolating the soluble α-glucan fiber composition from the product mixture comprising glucose oligomers; and

d. optionally concentrating the soluble α-glucan fiber composition.

In another embodiment, a method is provided to make a blended carbohydrate composition, the method comprising combining the soluble α-glucan fiber composition described above with one or more of the following: a monosaccharide, a disaccharide, glucose, sucrose, fructose, leucrose, corn syrup, high fructose corn syrup, isomerized sugar, maltose, trehalose, panose, raffinose, cellobiose, isomaltose, honey, maple sugar, a fruit-derived sweetener, sorbitol, maltitol, isomaltitol, lactose, nigerose, kojibiose, xylitol, erythritol, dihydrochalcone, stevioside, α-glycosyl stevioside, acesulfame potassium, alitame, neotame, glycyrrhizin, thaumantin, sucralose, L-aspartyl-L-phenylalanine methyl ester, saccharine, maltodextrin, starch, potato starch, tapioca starch, dextran, soluble corn fiber, a resistant maltodextrin, a branched maltodextrin, inulin, polydextrose, a fructooligosaccharide, a galactooligosaccharide, a xylooligosaccharide, an arabinoxylooligosaccharide, a nigerooligosaccharide, a gentiooligosaccharide, hemicellulose, fructose oligomer syrup, an isomaltooligosaccharide, a filler, an excipient, a binder, or any combination thereof.

In another embodiment, a method is provided to make a food product, the method comprising mixing one or more edible food ingredients with the present soluble α-glucan fiber composition as described above, a carbohydrate composition comprising the present soluble α-glucan fiber composition, or a combination thereof.

In another embodiment, a method to reduce the glycemic index of a food or beverage is provided, the method comprising incorporating into a food or beverage the present soluble α-glucan fiber composition.

In another embodiment, a method of inhibiting the elevation of blood-sugar level is provided, the method comprising a step of administering the soluble α-glucan fiber composition to a mammal.

In another embodiment, a method of lowering lipids in a living body is provided, the method comprising a step of administering the soluble α-glucan fiber composition to a mammal.

In another embodiment, a method of treating constipation is provided, the method comprising administering the soluble α-glucan fiber composition to a mammal.

In another embodiment, a method to alter fatty acid production in a mammalian colon is provided, the method comprising a step of administering an effective amount of the soluble α-glucan fiber composition to a mammal; preferably wherein the short chain fatty acid production is increased, the branched chain fatty acid production is decreased, or both.

In another embodiment, a cosmetic composition comprising the soluble α-glucan fiber composition is provided.

In another embodiment, a pharmaceutical composition comprising the soluble α-glucan fiber composition is provided.

In another embodiment, a low cariogenicity composition comprising the soluble α-glucan fiber composition and at least one polyol is provided.

In another embodiment, a use of the soluble α-glucan fiber composition in a food composition suitable for consumption by humans and animals is provided.

In another embodiment, a composition comprising 0.01 to 99 wt % (dry solids basis) of present soluble α-glucan fiber composition and at least one of the following ingredients: a synbiotic, a peptide, a peptide hydrolysate, a protein, a protein hydrolysate, a soy protein, a dairy protein, an amino acid, a polyol, a polyphenol, a vitamin, a mineral, an herbal, an herbal extract, a fatty acid, a polyunsaturated fatty acid (PUFAs), a phytosteroid, betaine, carotenoid, a digestive enzyme, a probiotic organism or any combination thereof is provided.

In another embodiment, a product produced by any of the above methods is provided.

### BRIEF DESCRIPTION OF THE BIOLOGICAL SEQUENCES

The following sequences comply with 37 C.F.R. §§ 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the European Patent Convention (EPC) and the Patent Cooperation Treaty (PCT) Rules 5.2 and 49.5(a-bis), and Section 208 and Annex C of the Administrative Instructions. The symbols and format used for nucleotide and amino acid sequence data comply with the rules set forth in 37 C.F.R. § 1.822.

SEQ ID NO: 1 is a polynucleotide sequence of a terminator sequence.

SEQ ID NO: 2 is a polynucleotide sequence of a linker sequence.

SEQ ID NO: 3 is the amino acid sequence of the Streptococcus salivarius Gtf-J glucosyltransferase as found in GENBANK® gi: 47527.

SEQ ID NO: 4 is the polynucleotide sequence encoding the Streptococcus salivarius mature Gtf-J glucosyltransferase.

SEQ ID NO: 5 is the amino acid sequence of Streptococcus salivarius Gtf-J mature glucosyltransferase (referred to herein as the “7527” glucosyltransferase” or “GTF7527”)).

SEQ ID NO: 6 is the amino acid sequence of Streptococcus salivarius Gtf-L glucosyltransferase as found in GENBANK® gi: 662379.

SEQ ID NO: 7 is the nucleic acid sequence encoding a truncated Streptococcus salivarius Gtf-L (GENBANK® gi: 662379) glucosyltransferase.

SEQ ID NO: 8 is the amino acid sequence of a truncated Streptococcus salivarius Gtf-L glucosyltransferase (also referred to herein as the “2379 glucosyltransferase” or “GTF2379”).

SEQ ID NO: 9 is the amino acid sequence of the Streptococcus mutans NN2025 Gtf-B glucosyltransferase as found in GENBANK® gi: 290580544.

SEQ ID NO: 10 is the nucleic acid sequence encoding a truncated Streptococcus mutans NN2025 Gtf-B (GENBANK® gi: 290580544) glucosyltransferase.

SEQ ID NO: 11 is the amino acid sequence of a truncated Streptococcus mutans NN2025 Gtf-B glucosyltransferase (also referred to herein as the “0544 glucosyltransferase” or “GTF0544”).

SEQ ID NOs: 12-13 are the nucleic acid sequences of primers.

SEQ ID NO: 14 is the amino acid sequence of the Streptococcus sobrinus Gtf-I glucosyltransferase as found in GENBANK® gi: 450874.

SEQ ID NO: 15 is the nucleic acid sequence encoding a truncated Streptococcus sobrinus Gtf-I (GENBANK® gi: 450874) glucosyltransferase.

SEQ ID NO: 16 is the amino acid sequence of a truncated Streptococcus sobrinus Gtf-I glucosyltransferase (also referred to herein as the “0874 glucosyltransferase” or “GTF0874”).

SEQ ID NO: 17 is the amino acid sequence of the Streptococcus sp. C150 Gtf-S glucosyltransferase as found in GENBANK® gi: 495810459 (previously known as GENBANK® gi:. 322373279)

SEQ ID NO: 18 is the nucleic acid sequence encoding a truncated Streptococcus sp. C150 gtf-S (GENBANK® gi: 495810459) glucosyltransferase.

SEQ ID NO: 19 is the amino acid sequence of a truncated Streptococcus sp. C150 Gtf-S glucosyltransferase (also referred to herein as the “0459 glucosyltransferase”, “GTF0459”, “3279 glucosyltransferase” or “GTF3279”).

SEQ ID NO: 20 is the nucleic acid sequence encoding the Paenibacillus humicus mutanase (GENBANK® gi: 257153265 where GENBANK® gi: 257153264 is the corresponding polynucleotide sequence) used in Example 12 for expression in E. coli BL21(DE3).

SEQ ID NO: 21 is the amino acid sequence of the mature Paenibacillus humicus mutanase (GENBANK® gi: 257153264; referred to herein as the “3264 mutanase” or “MUT3264”) used in Example 12 for expression in E. coli BL21(DE3).

SEQ ID NO: 22 is the amino acid sequence of the Paenibacillus humicus mutanase as found in GENBANK® gi: 257153264).

SEQ ID NO: 23 is the nucleic acid sequence encoding the Paenibacillus humicus mutanase used in Example 13 for expression in B. subtilis host BG6006.

SEQ ID NO: 24 is the amino acid sequence of the mature Paenibacillus humicus mutanase used in Example 13 for expression in B. subtilis host BG6006. As used herein, this mutanase may also be referred to herein as “MUT3264”.

SEQ ID NO: 25 is the amino acid sequence of the B. subtilis AprE signal peptide used in the expression vector that was coupled to various enzymes for expression in B. subtilis.

SEQ ID NO: 26 is the nucleic acid sequence encoding the Penicillium marneffei ATCC® 18224™ mutanase.

SEQ ID NO: 27 is the amino acid sequence of the Penicillium marneffei ATCC® 18224™ mutanase (GENBANK® gi: 212533325; also referred to herein as the “3325 mutanase” or “MUT3325”).

SEQ ID NO: 28 is the nucleic acid sequence encoding the Aspergillus nidulans FGSC A4 mutanase.

SEQ ID NO: 29 is the amino acid sequence of the Aspergillus nidulans FGSC A4 mutanase (GENBANK® gi: 259486505; also referred to herein as the “6505 mutanase” or “MUT6505”).

SEQ ID NOs: 30-52 are the nucleic acid sequences of various primers used in Example 17.

SEQ ID NO: 53 is the nucleic acid sequence encoding a Hypocrea tawa mutanase.

SEQ ID NO: 54 is the amino acid sequence of the Hypocrea tawa mutanase as disclosed in U.S. Patent Appl. Pub. No. 2011-0223117A1 (also referred to herein as the “H. tawa mutanase”).

SEQ ID NO: 55 is the nucleic acid sequence encoding the Trichoderma konilangbra mutanase.

SEQ ID NO: 56 is the amino acid sequence of the Trichoderma konilangbra mutanase as disclosed in U.S. Patent Appl. Pub. No. 2011-0223117A1 (also referred to herein as the “T. konilangbra mutanase”).

SEQ ID NO: 57 is the nucleic acid sequence encoding the Trichoderma reesei RL-P37 mutanase.

SEQ ID NO: 58 is the amino acid sequence of the Trichoderma reesei RL-P37 mutanase as disclosed in U.S. Patent Appl. Pub. No. 2011-0223117A1 (also referred to herein as the “T. reesei 592 mutanase”).

SEQ ID NO: 59 is the polynucleotide sequence of plasmid pTrex3.

SEQ ID NO: 60 is the nucleic acid sequence encoding a truncated Streptococcus oralis glucosyltransferase (GENBANK® gi:7684297).

SEQ ID NO: 61 is the amino acid sequence of the truncated Streptococcus oralis glucosyltransferase encoded by SEQ ID NO: 60, and which is referred to herein as “GTF4297”.

SEQ ID NO: 62 is the nucleic acid sequence encoding a truncated version of a Streptococcus mutans glucosyltransferase (GENBANK® gi:3130088).

SEQ ID NO: 63 is the amino acid sequence of the truncated Streptococcus mutans glucosyltransferase encoded by SEQ ID NO: 62, which is referred to herein as “GTF0088”.

SEQ ID NO: 64 is the nucleic acid sequence encoding a truncated version of a Streptococcus mutans glucosyltransferase (GENBANK® gi:24379358).

SEQ ID NO: 65 is the amino acid sequence of the truncated Streptococcus mutans glucosyltransferase encoded by SEQ ID NO: 64, which is referred to herein as “GTF9358”.

SEQ ID NO: 66 is the nucleic acid sequence encoding a truncated version of a Streptococcus gallolyticus glucosyltransferase (GENBANK® gi:32597842).

SEQ ID NO: 67 is the amino acid sequence of the truncated Streptococcus gallolyticus glucosyltransferase encoded by SEQ ID NO: 66, which is referred to herein as “GTF7842”.

SEQ ID NO: 68 is the amino acid sequence of a Lactobacillus reuteri glucosyltransferase as found in GENBANK® gi:51574154.

SEQ ID NO: 69 is the nucleic acid sequence encoding a truncated version of the Lactobacillus reuteri glucosyltransferase (GENBANK® gi:51574154).

SEQ ID NO: 70 is the amino acid sequence of the truncated Lactobacillus reuteri glucosyltransferase encoded by SEQ ID NO: 69, which is referred to herein as “GTF4154”.

SEQ ID NO: 71 is the amino acid sequence of a Streptococcus downei GTF-S glucosyltransferase as found in GENBANK® gi: 121729 (precursor with the native signal sequence) also referred to herein as “GTF1729”.

SEQ ID NO: 72 is the amino acid sequence of a Streptococcus criceti HS-6 GTF-S glucosyltransferase as found in GENBANK® gi: 357235604 (precursor with the native signal sequence) also referred to herein as “GTF5604”. The same amino acid sequence is reported under GENBANK® gi:4691428 for a glucosyltransferase from Streptococcus criceti. As such, this particular amino acid sequence is also referred to herein as “GTF1428”.

SEQ ID NO: 73 is the amino acid sequence of a Streptococcus criceti HS-6 glucosyltransferase derived from GENBANK® gi: 357236477 (also referred to herein as “GTF6477”) where the native signal sequence was substituted with the AprE signal sequence for expression in Bacillus subtilis.

SEQ ID NO: 74 is the amino acid sequence of a Streptococcus criceti HS-6 glucosyltransferase derived from GENBANK® gi: 357236477 (also referred to herein as “GTF6477-V1” or “357236477-V1”) where the native signal sequence was substituted with the AprE signal sequence for expression in Bacillus subtilis and contains a single amino acid substitution.

SEQ ID NO: 75 is the amino acid sequence of a Streptococcus salivarius M18 glucosyltransferase derived from GENBANK® gi: 345526831(also referred to herein as “GTF6831”) where the native signal sequence was substituted with the AprE signal sequence for expression in Bacillus subtilis.

SEQ ID NO: 76 is the amino acid sequence of a Lactobacillus animalis KCTC 3501 glucosyltransferase derived from GENBANK® gi: 335358117 (also referred to herein as “GTF8117”) where the native signal sequence was substituted with the AprE signal sequence for expression in Bacillus subtilis.

SEQ ID NO: 77 is the amino acid sequence of a Streptococcus gordonii glucosyltransferase derived from GENBANK® gi: 1054877 (also referred to herein as “GTF4877”) where the native signal sequence was substituted with the AprE signal sequence for expression in Bacillus subtilis.

SEQ ID NO: 78 is the amino acid sequence of a Streptococcus sobrinus glucosyltransferase derived from GENBANK® gi: 22138845 (also referred to herein as “GTF8845”) where the native signal sequence was substituted with the AprE signal sequence for expression in Bacillus subtilis.

SEQ ID NO: 79 is the amino acid sequence of the Streptococcus downei glucosyltransferase as found in GENBANK® gi: 121724.

SEQ ID NO: 80 is the nucleic acid sequence encoding a truncated Streptococcus downei (GENBANK® gi: 121724) glucosyltransferase.

SEQ ID NO: 81 is the amino acid sequence of the truncated Streptococcus downei glucosyltransferase encoded by SEQ ID NO: 80 (also referred to herein as the “1724 glucosyltransferase” or “GTF1724”).

SEQ ID NO: 82 is the amino acid sequence of the Streptococcus dentirousetti glucosyltransferase as found in GENBANK® gi: 167735926.

SEQ ID NO: 83 is the nucleic acid sequence encoding a truncated Streptococcus dentirousetti (GENBANK® gi: 167735926) glucosyltransferase.

SEQ ID NO: 84 is the amino acid sequence of the truncated Streptococcus dentirousetti glucosyltransferase encoded by SEQ ID NO: 83 (also referred to herein as the “5926 glucosyltransferase” or “GTF5926”).

SEQ ID NO: 85 is the amino acid sequence of the dextran dextrinase (EC 2.4.1.2) expressed by a strain Gluconobacter oxydans referred to herein as “DDase” (see JP2007181452(A)).

SEQ ID NO: 86 is the nucleic acid sequence encoding the GTF0459 amino acid sequence of SEQ ID NO: 19.

SEQ ID NO: 87 is the nucleic acid sequence encoding a truncated form of GTF0470, a GTF0459 homolog.

SEQ ID NO: 88 is the amino acid sequence encoded by SEQ ID NO: 87.

SEQ ID NO: 89 is the nucleic acid sequence encoding a truncated form of GTF07317, a GTF0459 homolog.

SEQ ID NO: 90 is the amino acid sequence encoded by SEQ ID NO: 89.

SEQ ID NO: 91 is the nucleic acid sequence encoding a truncated form of GTF1645, a GTF0459 homolog.

SEQ ID NO: 92 is the amino acid sequence encoded by SEQ ID NO: 91.

SEQ ID NO: 93 is the nucleic acid sequence encoding a truncated form of GTF6099, a GTF0459 homolog.

SEQ ID NO: 94 is the amino acid sequence encoded by SEQ ID NO: 93.

SEQ ID NO: 95 is the nucleic acid sequence encoding a truncated form of GTF8467, a GTF0459 homolog.

SEQ ID NO: 96 is the amino acid sequence encoded by SEQ ID NO: 95.

SEQ ID NO: 97 is the nucleic acid sequence encoding a truncated form of GTF8487, a GTF0459 homolog.

SEQ ID NO: 98 is the amino acid sequence encoded by SEQ ID NO: 97.

SEQ ID NO: 99 is the nucleic acid sequence encoding a truncated form of GTF06549, a GTF0459 homolog.

SEQ ID NO: 100 is the amino acid sequence encoded by SEQ ID NO: 99.

SEQ ID NO: 101 is the nucleic acid sequence encoding a truncated form of GTF3879, a GTF0459 homolog.

SEQ ID NO: 102 is the amino acid sequence encoded by SEQ ID NO: 101.

SEQ ID NO: 103 is the nucleic acid sequence encoding a truncated form of GTF4336, a GTF0459 homolog.

SEQ ID NO: 104 is amino acid sequence encoded by SEQ ID NO: 103.

SEQ ID NO: 105 is the nucleic acid sequence encoding a truncated form of GTF4491, a GTF0459 homolog.

SEQ ID NO: 106 is the amino acid sequence encoded by SEQ ID NO: 105.

SEQ ID NO: 107 is the nucleic acid sequence encoding a truncated form of GTF3808, a GTF0459 homolog.

SEQ ID NO: 108 is the amino acid sequence encoded by SEQ ID NO: 107.

SEQ ID NO: 109 is the nucleic acid sequence encoding a truncated form of GTF0974, a GTF0459 homolog.

SEQ ID NO: 110 is the amino acid sequence encoded by SEQ ID NO: 109.

SEQ ID NO: 111 is the nucleic acid sequence encoding a truncated form of GTF0060, a GTF0459 homolog.

SEQ ID NO: 112 is the amino acid sequence encoded by SEQ ID NO: 111.

SEQ ID NO: 113 is the nucleic acid sequence encoding a truncated form of GTF0487, a GTF0459 non-homolog.

SEQ ID NO: 114 is the amino acid sequence encoded by SEQ ID NO: 113.

SEQ ID NO: 115 is the nucleic acid sequence encoding a truncated form of GTF5360, a GTF0459 non-homolog.

SEQ ID NO: 116 is the amino acid sequence encoded by SEQ ID NO: 115.

SEQ ID NOs: 117, 119, 121, and 123 are nucleotide sequences encoding T5 C-terminal truncations of GTF0974, GTF4336, GTF4491, and GTF3808, respectively.

SEQ ID NOs: 118, 120, 122, and 124 are amino acid sequences of T5 C-terminal truncations of GTF0974, GTF4336, GTF4491, and GTF3808, respectively.

SEQ ID NO: 125 is the nucleotide sequence encoding a T5 C-terminal truncation of GTF0459.

SEQ ID NO: 126 is the amino acid sequence encoded by the nucleotide sequence of SEQ ID NO: 125.

SEQ ID NO: 127 is the nucleotide sequence encoding a T4 C-terminal truncation of GTF0974.

SEQ ID NO: 128 is the amino acid sequence encoded by the nucleotide sequence of SEQ ID NO: 127.

SEQ ID NO: 129 is the nucleotide sequence encoding a T4 C-terminal truncation of GTF4336.

SEQ ID NO: 130 is the amino acid sequence encoded by the nucleotide sequence of SEQ ID NO: 129.

SEQ ID NO: 131 is the nucleotide sequence encoding a T4 C-terminal truncation of GTF4491.

SEQ ID NO: 132 is the amino acid sequence encoded by the nucleotide sequence of SEQ ID NO: 131.

SEQ ID NO: 133 is the nucleotide sequence encoding a T6 C-terminal truncation of GTF0459.

SEQ ID NO: 134 is the amino acid sequence encoded by SEQ ID NO: 133.

SEQ ID NO: 135 is the nucleotide sequence encoding a T1 C-terminal truncation of GTF0974.

SEQ ID NO: 136 is the amino acid sequence encoded by SEQ ID NO: 135.

SEQ ID NO: 137 is the nucleotide sequence encoding a T2 C-terminal truncation of GTF0974.

SEQ ID NO: 138 is the amino acid sequence encoded by SEQ ID NO: 137.

SEQ ID NO: 139 is the nucleotide sequence encoding a T6 C-terminal truncation of GTF0974.

SEQ ID NO: 140 is the amino acid sequence encoded by SEQ ID NO: 139.

SEQ ID NO: 141 is the nucleotide sequence encoding a T1 C-terminal truncation of GTF4336.

SEQ ID NO: 142 is the amino acid sequence encoded by SEQ ID NO: 141.

SEQ ID NO: 143 is the nucleotide sequence encoding a T2 C-terminal truncation of GTF4336.

SEQ ID NO: 144 is the amino acid sequence encoded by SEQ ID NO; 143.

SEQ ID NO: 145 is the nucleotide sequence encoding a T6 C-terminal truncation of GTF4336.

SEQ ID NO: 146 is the amino acid sequence encoded by SEQ ID NO: 145.

SEQ ID NO: 147 is the nucleotide sequence encoding a T1 C-terminal truncation of GTF4991.

SEQ ID NO: 148 is the amino acid sequence encoded by SEQ ID NO: 147.

SEQ ID NO: 149 is the nucleotide sequence encoding a T2 C-terminal truncation of GTF4991.

SEQ ID NO: 150 is the amino acid sequence encoded by SEQ ID NO: 149.

SEQ ID NO: 151 is the nucleotide sequence encoding a T6 C-terminal truncation of GTF4991.

SEQ ID NO: 152 is the amino acid sequence encoded by SEQ ID NO: 151.

SEQ ID NO: 153 is an amino acid consensus sequence based on the alignment of GTF0459 and its identified homologs.

### DETAILED DESCRIPTION OF THE INVENTION

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein, the term “obtainable from” shall mean that the source material (for example, sucrose) is capable of being obtained from a specified source, but is not necessarily limited to that specified source.

As used herein, the term “effective amount” will refer to the amount of the substance used or administered that is suitable to achieve the desired effect. The effective amount of material may vary depending upon the application. One of skill in the art will typically be able to determine an effective amount for a particular application or subject without undo experimentation.

As used herein, the term “isolated” means a substance in a form or environment that does not occur in nature. Non-limiting examples of isolated substances include (1) any non-naturally occurring substance, (2) any substance including, but not limited to, any host cell, enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated.

As used herein, the terms “very slow to no digestibility”, “little or no digestibility”, and “low to no digestibility” will refer to the relative level of digestibility of the soluble glucan fiber as measured by the Association of Official Analytical Chemists International (AOAC) method 2009.01 (“AOAC 2009.01”; McCleary et al. (2010) J. AOAC Int., 93(1), 221-233); where little or no digestibility will mean less than 12% of the soluble glucan fiber composition is digestible, preferably less than 5% digestible, more preferably less than 1% digestible on a dry solids basis (d.s.b.). In another aspect, the relative level of digestibility may be alternatively be determined using AOAC 2011.25 (Integrated Total Dietary Fiber Assay) (McCleary et al., (2012) J. AOAC Int., 95 (3), 824-844.

As used herein, term “water soluble” will refer to the present glucan fiber composition comprised of fibers that are soluble at 20 wt % or higher in pH 7 water at 25° C.

As used herein, the terms “soluble fiber”, “soluble glucan fiber”, “α-glucan fiber”, “cane sugar fiber”, “glucose fiber”, “beet sugar fiber”, “soluble dietary fiber”, and “soluble glucan fiber composition” refer to the present fiber composition comprised of water soluble glucose oligomers having a glucose polymerization degree of 3 or more that is digestion resistant (i.e., exhibits very slow to no digestibility) with little or no absorption in the human small intestine and is at least partially fermentable in the lower gasterointestinal tract. Digestibility of the soluble glucan fiber composition is measured using AOAC method 2009.01. The present soluble glucan fiber composition is enzymatically synthesized from sucrose (α-D-Glucopyranosyl β-D-fructofuranoside; CAS #57-50-1) obtainable from, for example, sugarcane and/or sugar beets. In one embodiment, the present soluble α-glucan fiber composition is not alternan or maltoalternan oligosaccharide.

As used herein, “weight average molecular weight” or “M_{w}” is calculated as M_{w}=ΣNᵢMᵢ²/ΣNᵢMᵢ; where Mᵢ is the molecular weight of a chain and Nᵢ is the number of chains of that molecular weight. The weight average molecular weight can be determined by technics such as static light scattering, small angle neutron scattering, X-ray scattering, and sedimentation velocity.

As used herein, “number average molecular weight” or “Mₙ” refers to the statistical average molecular weight of all the polymer chains in a sample. The number average molecular weight is calculated as Mₙ=ΣNᵢMᵢ/ΣNᵢ where Mᵢ is the molecular weight of a chain and Nᵢ is the number of chains of that molecular weight. The number average molecular weight of a polymer can be determined by technics such as gel permeation chromatography, viscometry via the (Mark-Houwink equation), and colligative methods such as vapor pressure osmometry, end-group determination or proton NMR.

As used herein, “polydispersity index”, “PDI”, “heterogeneity index”, and “dispersity” refer to a measure of the distribution of molecular mass in a given polymer (such as a glucose oligomer) sample and can be calculated by dividing the weight average molecular weight by the number average molecular weight (PDI=M_{w}/Mₙ).

It shall be noted that the terms “glucose” and “glucopyranose” as used herein are considered as synonyms and used interchangeably. Similarly the terms “glucosyl” and “glucopyranosyl” units are used herein are considered as synonyms and used interchangeably.

As used herein, “glycosidic linkages” or “glycosidic bonds” will refer to the covalent the bonds connecting the sugar monomers within a saccharide oligomer (oligosaccharides and/or polysaccharides). Example of glycosidic linkage may include α-linked glucose oligomers with 1,6-α-D-glycosidic linkages (herein also referred to as α-D-(1,6) linkages or simply “α-(1,6)” linkages); 1,3-α-D-glycosidic linkages (herein also referred to as α-D-(1,3) linkages or simply “α-(1,3)” linkages; 1,4-α-D-glycosidic linkages (herein also referred to as α-D-(1,4) linkages or simply “α-(1,4)” linkages; 1,2-α-D-glycosidic linkages (herein also referred to as α-D-(1,2) linkages or simply “α-(1,2)” linkages; and combinations of such linkages typically associated with branched saccharide oligomers.

As used herein, the terms “glucansucrase”, “glucosyltransferase”, “glucoside hydrolase type 70”, “GTF”, and “GS” will refer to transglucosidases classified into family 70 of the glycoside-hydrolases typically found in lactic acid bacteria such as Streptococcus, Leuconostoc, Weisella or Lactobacillus genera (see Carbohydrate Active Enames database; “CAZy”; Cantarel et al., (2009) Nucleic Acids Res 37:D233-238). The GTF enzymes are able to polymerize the D-glucosyl units of sucrose to form homooligosaccharides or homopolysaccharides. Glucosyltransferases can be identified by characteristic structural features such as those described in Leemhuis et al. (J. Biotechnology (2013) 162:250-272) and Monchois et al. (FEMS Micro. Revs. (1999) 23:131-151). Depending upon the specificity of the GTF enzyme, linear and/or branched glucans comprising various glycosidic linkages may be formed such as α-(1,2), α-(1,3), α-(1,4) and α-(1,6). Glucosyltransferases may also transfer the D-glucosyl units onto hydroxyl acceptor groups. A non-limiting list of acceptors include carbohydrates, alcohols, polyols or flavonoids. Specific acceptors may also include maltose, isomaltose, isomaltotriose, and methyl-α-D-glucan. The structure of the resultant glucosylated product is dependent upon the enzyme specificity. A non-limiting list of glucosyltransferase sequences is provided as amino acid SEQ ID NOs: 3, 5, 6, 8, 9, 11, 14, 16, 17, 19, 61, 63, 65, 67, 68, 70, 72, 73, 74, 75, 76, 77, 78, 79, 81, 82, 84, 88, 90, 92, 94, 96, 98, 100, 102, 104, 106, 108, 110, and 112. In one aspect, the glucosyltransferase is expressed in a truncated and/or mature form. Non-limiting examples of truncated glucosyltransferase amino acid sequences include SEQ ID NOs: 118, 120, 122, 124, 126, 128, 130, 132, 134, 136, 138, 140, 142, 144, 146, 148, 150, and 152.

As used herein, the term “isomaltooligosaccharide” or “IMO” refers to glucose oligomers comprised essentially of α-D-(1,6) glycosidic linkage typically having an average size of DP 2 to 20. Isomaltooligosaccharides can be produced commercially from an enzymatic reaction of α-amylase, pullulanase, β-amylase, and α-glucosidase upon corn starch or starch derivative products. Commercially available products comprise a mixture of isomaltooligosaccharides (DP ranging from 3 to 8, e.g., isomaltotriose, isomaltotetraose, isomaltopentaose, isomaltohexaose, isomaltoheptaose, isomaltooctaose) and may also include panose.

As used herein, the term “dextran” refers to water soluble α-glucans comprising at least 95% α-D-(1,6) glycosidic linkages (typically with up to 5% α-D-(1,3) glycosidic linkages at branching points) that are more than 10% digestible as measured by the Association of Official Analytical Chemists International (AOAC) method 2009.01 (“AOAC 2009.01”). Dextrans often have an average molecular weight above 1000 kDa. As used herein, enzymes capable of synthesizing dextran from sucrose may be described as “dextransucrases” (EC 2.4.1.5).

As used herein, the term “mutan” refers to water insoluble α-glucans comprised primarily (50% or more of the glycosidic linkages present) of 1,3-α-D glycosidic linkages and typically have a degree of polymerization (DP) that is often greater than 9. Enzymes capable of synthesizing mutan or α-glucan oligomers comprising greater than 50% 1,3-α-D glycosidic linkages from sucrose may be described as “mutansucrases” (EC 2.4.1.-) with the proviso that the enzyme does not produce alternan.

As used herein, the term “alternan” refers to α-glucans having alternating 1,3-α-D glycosidic linkages and 1,6-α-D glycosidic linkages over at least 50% of the linear oligosaccharide backbone. Enzymes capable of synthesizing alternan from sucrose may be described as “alternansucrases” (EC 2.4.1.140).

As used herein, the term “reuteran” refers to soluble α-glucan comprised 1,4-α-D-glycosidic linkages (typically >50%); 1,6-α-D-glycosidic linkages; and 4,6-disubstituted α-glucosyl units at the branching points. Enzymes capable of synthesizing reuteran from sucrose may be described as “reuteransucrases” (EC 2.4.1.-).

As used herein, the terms “α-glucanohydrolase” and “glucanohydrolase” will refer to an enzyme capable of hydrolyzing an α-glucan oligomer. As used herein, the glucanohydrolase may be defined by the endohydrolysis activity towards certain α-D-glycosidic linkages. Examples may include, but are not limited to, dextranases (EC 3.2.1.11; capable of endohydrolyzing α-(1,6)-linked glycosidic bonds), mutanases (EC 3.2.1.59; capable of endohydrolyzing α-(1,3)-linked glycosidic bonds), and alternanases (EC 3.2.1.-; capable of endohydrolytically cleaving alternan). Various factors including, but not limited to, level of branching, the type of branching, and the relative branch length within certain α-glucans may adversely impact the ability of an α-glucanohydrolase to endohydrolyze some glycosidic linkages.

As used herein, the term “dextranase” (α-1,6-glucan-6-glucanohydrolase; EC 3.2.1.11) refers to an enzyme capable of endohydrolysis of 1,6-α-D-glycosidic linkages (the linkage predominantly found in dextran). Dextranases are known to be useful for a number of applications including the use as ingredient in dentifrice for prevent dental caries, plaque and/or tartar and for hydrolysis of raw sugar juice or syrup of sugar canes and sugar beets. Several microorganisms are known to be capable of producing dextranases, among them fungi of the genera Penicillium, Paecilomyces, Aspergillus, Fusarium, Spicaria, Verticillium, Helminthosporium and Chaetomium; bacteria of the genera Lactobacillus, Streptococcus, Cellvibrio, Cytophaga, Brevibacterium, Pseudomonas, Corynebacterium, Arthrobacter and Flavobacterium, and yeasts such as Lipomyces starkeyi. Food grade dextranases are commercially available. An example of a food grade dextrinase is DEXTRANASE® Plus L, an enzyme from Chaetomium erraticum sold by Novozymes A/S, Bagsvaerd, Denmark.

As used herein, the term “mutanase” (glucan endo-1,3-α-glucosidase; EC 3.2.1.59) refers to an enzyme which hydrolytically cleaves 1,3-α-D-glycosidic linkages (the linkage predominantly found in mutan). Mutanases are available from a variety of bacterial and fungal sources. A non-limiting list of mutanases is provided as amino acid sequences 21, 22, 24, 27, 29, 54, 56, and 58.

As used herein, the term “alternanase” (EC 3.2.1.-) refers to an enzyme which endo-hydrolytically cleaves alternan (U.S. Pat. No. 5,786,196 to Cote et al.).

As used herein, the term “wild type enzyme” will refer to an enzyme (full length and active truncated forms thereof) comprising the amino acid sequence as found in the organism from which was obtained and/or annotated. The enzyme (full length or catalytically active truncation thereof) may be recombinantly produced in a microbial host cell. The enzyme is typically purified prior to being used as a processing aid in the production of the present soluble α-glucan fiber composition. In one aspect, a combination of at least two wild type enzymes simultaneously present in the reaction system is used in order to obtain the present soluble glucan fiber composition. In another aspect, under certain reaction conditions (for example, a reaction temperature around 47° C. to 50° C.) it may be possible to use a single wild type glucosyltransferase to produce the soluble glucan fiber disclosed herein (see Examples 38, 44, and 45). In another aspect, the present method comprises a single reaction chamber comprising at least one glucosyltransferase capable of forming a soluble α-glucan fiber composition comprising 50% or more α-(1,3) glycosidic linkages (such as a mutansucrase) and at least one α-glucanohydrolase having endohydrolysis activity for the α-glucan synthesized from the glucosyltransferase(s) present in the reaction system.

As used herein, the terms “substrate” and “suitable substrate” will refer to a composition comprising sucrose. In one embodiment, the substrate composition further comprises one or more suitable acceptors, such as maltose, isomaltose, isomaltotriose, and methyl-α-D-glucan, to name a few. In one embodiment, a combination of at least one glucosyltransferase capable of forming glucose oligomers is used in combination with at least one α-glucanohydrolase in the same reaction mixture (i.e., they are simultaneously present and active in the reaction mixture). As such, the “substrate” for the α-glucanohydrolase is the glucose oligomers concomitantly being synthesized in the reaction mixture by the glucosyltransferase from sucrose. In one aspect, a two-enzyme method (i.e., at least one glucosyltransferase (GTF) and at least one α-glucanohydrolase) where the enzymes are not used concomitantly in the reaction mixture is excluded, by proviso, from the methods disclosed herein.

As used herein, the terms “suitable enzymatic reaction mixture”, “suitable reaction components”, “suitable aqueous reaction mixture”, and “reaction mixture”, refer to the materials (suitable substrate(s)) and water in which the reactants come into contact with the enzyme(s). The suitable reaction components may be comprised of a plurality of enzymes. In one aspect, the suitable reaction components comprises at least one glucansucrase enzyme. In a further aspect, the suitable reaction components comprise at least one glucansucrase and at least one α-glucanohydrolase.

As used herein, “one unit of glucansucrase activity” or “one unit of glucosyltransferase activity” is defined as the amount of enzyme required to convert 1 μmol of sucrose per minute when incubated with 200 g/L sucrose at pH 5.5 and 37° C. The sucrose concentration was determined using HPLC.

As used herein, “one unit of dextranase activity” is defined as the amount of enzyme that forms 1 μmol reducing sugar per minute when incubated with 0.5 mg/mL dextran substrate at pH 5.5 and 37° C. The reducing sugars were determined using the PAHBAH assay (Lever M., (1972), A New Reaction for Colorimetric Determination of Carbohydrates, Anal. Biochem. 47, 273-279).

As used herein, “one unit of mutanase activity” is defined as the amount of enzyme that forms 1 μmol reducing sugar per minute when incubated with 0.5 mg/mL mutan substrate at pH 5.5 and 37° C. The reducing sugars were determined using the PAHBAH assay (Lever M., supra).

As used herein, the term “enzyme catalyst” refers to a catalyst comprising an enzyme or combination of enzymes having the necessary activity to obtain the desired soluble glucan fiber composition. In certain embodiments, a combination of enzyme catalysts may be required to obtain the desired soluble glucan fiber composition. The enzyme catalyst(s) may be in the form of a whole microbial cell, permeabilized microbial cell(s), one or more cell components of a microbial cell extract(s), partially purified enzyme(s) or purified enzyme(s). In certain embodiments the enzyme catalyst(s) may also be chemically modified (such as by pegylation or by reaction with cross-linking reagents). The enzyme catalyst(s) may also be immobilized on a soluble or insoluble support using methods well-known to those skilled in the art; see for example, Immobilization of Enzymes and Cells; Gordon F. Bickerstaff, Editor; Humana Press, Totowa, N.J., USA; 1997.

As used herein, “pharmaceutically-acceptable” means that the compounds or compositions in question are suitable for use in contact with the tissues of humans and other animals without undue toxicity, incompatibility, instability, irritation, allergic response, and the like, commensurate with a reasonable benefit/risk ratio.

As used herein, the term “oligosaccharide” refers to homopolymers containing between 3 and about 30 monosaccharide units linked by α-glycosidic bonds.

As used herein the term “polysaccharide” refers to homopolymers containing greater than 30 monosaccharide units linked by α-glycosidic bonds.

As used herein, the term “food” is used in a broad sense herein to include a variety of substances that can be ingested by humans including, but not limited to, beverages, dairy products, baked goods, energy bars, jellies, jams, cereals, dietary supplements, and medicinal capsules or tablets.

As used herein, the term “pet food” or “animal feed” is used in a broad sense herein to include a variety of substances that can be ingested by nonhuman animals and may include, for example, dog food, cat food, and feed for livestock.

A “subject” is generally a human, although as will be appreciated by those skilled in the art, the subject may be a non-human animal. Thus, other subjects may include mammals, such as rodents (including mice, rats, hamsters and guinea pigs), cats, dogs, rabbits, cows, horses, goats, sheep, pigs, and primates (including monkeys, chimpanzees, orangutans and gorillas).

The term “cholesterol-related diseases”, as used herein, includes but is not limited to conditions which involve elevated levels of cholesterol, in particular non-high density lipid (non-HDL) cholesterol in plasma, e.g., elevated levels of LDL cholesterol and elevated HDL/LDL ratio, hypercholesterolemia, and hypertriglyceridemia, among others. In patients with hypercholesteremia, lowering of LDL cholesterol is among the primary targets of therapy. In patients with hypertriglyceridemia, lower high serum triglyceride concentrations are among the primary targets of therapy. In particular, the treatment of cholesterol-related diseases as defined herein comprises the control of blood cholesterol levels, blood triglyceride levels, blood lipoprotein levels, blood glucose, and insulin sensitivity by administering the present glucan fiber or a composition comprising the present glucan fiber.

As used herein, “personal care products” means products used in the cosmetic treatment hair, skin, scalp, and teeth, including, but not limited to shampoos, body lotions, shower gels, topical moisturizers, toothpaste, tooth gels, mouthwashes, mouthrinses, anti-plaque rinses, and/or other topical treatments. In some particularly preferred embodiments, these products are utilized on humans, while in other embodiments, these products find cosmetic use with non-human animals (e.g., in certain veterinary applications).

As used herein, the terms “isolated nucleic acid molecule”, “isolated polynucleotide”, and “isolated nucleic acid fragment” will be used interchangeably and refer to a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or as | Xaa | X |
| defined herein | | |

It would be recognized by one of ordinary skill in the art that modifications of amino acid sequences disclosed herein can be made while retaining the function associated with the disclosed amino acid sequences. For example, it is well known in the art that alterations in a gene which result in the production of a chemically equivalent amino acid at a given site may not affect the functional properties of the encoded protein. For example, any particular amino acid in an amino acid sequence disclosed herein may be substituted for another functionally equivalent amino acid. For the purposes of the present invention substitutions are defined as exchanges within one of the following five groups:
1. Small aliphatic, nonpolar or slightly polar residues: Ala, Ser, Thr (Pro, Gly);
2. Polar, negatively charged residues and their amides: Asp, Asn, Glu, Gln;
3. Polar, positively charged residues: His, Arg, Lys;
4. Large aliphatic, nonpolar residues: Met, Leu, Ile, Val (Cys); and
5. Large aromatic residues: Phe, Tyr, and Trp.
   Thus, a codon for the amino acid alanine, a hydrophobic amino acid, may be substituted by a codon encoding another less hydrophobic residue (such as glycine) or a more hydrophobic residue (such as valine, leucine, or isoleucine). Similarly, changes which result in substitution of one negatively charged residue for another (such as aspartic acid for glutamic acid) or one positively charged residue for another (such as lysine for arginine) can also be expected to produce a functionally equivalent product. In many cases, nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products.

As used herein, the term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes.

As used herein, “synthetic genes” can be assembled from oligonucleotide building blocks that are chemically synthesized using procedures known to those skilled in the art. These building blocks are ligated and annealed to form gene segments that are then enzymatically assembled to construct the entire gene. “Chemically synthesized”, as pertaining to a DNA sequence, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequences to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

As used herein, “gene” refers to a nucleic acid molecule that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may include regulatory sequences and coding sequences that are derived from different sources, or regulatory sequences and coding sequences derived from the same source, but arranged in a manner different from that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

As used herein, “coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding sites, and stem-loop structures.

As used herein, the term “operably linked” refers to the association of nucleic acid sequences on a single nucleic acid molecule so that the function of one is affected by the other. For example, a promoter is operably linked with a coding sequence when it is capable of affecting the expression of that coding sequence, i.e., the coding sequence is under the transcriptional control of the promoter. Coding sequences can be operably linked to regulatory sequences in sense or antisense orientation.

As used herein, the term “expression” refers to the transcription and stable accumulation of sense (mRNA) or antisense RNA derived from the nucleic acid molecule of the invention. Expression may also refer to translation of mRNA into a polypeptide.

As used herein, “transformation” refers to the transfer of a nucleic acid molecule into the genome of a host organism, resulting in genetically stable inheritance. In the present invention, the host cell's genome includes chromosomal and extrachromosomal (e.g., plasmid) genes. Host organisms containing the transformed nucleic acid molecules are referred to as “transgenic”, “recombinant” or “transformed” organisms.

As used herein, the term “sequence analysis software” refers to any computer algorithm or software program that is useful for the analysis of nucleotide or amino acid sequences. “Sequence analysis software” may be commercially available or independently developed. Typical sequence analysis software will include, but is not limited to, the GCG suite of programs (Wisconsin Package Version 9.0, Accelrys Software Corp., San Diego, Calif.), BLASTP, BLASTN, BLASTX (Altschul et al., J. Mol. Biol. 215:403-410 (1990)), and DNASTAR (DNASTAR, Inc. 1228 S. Park St. Madison, Wis. 53715 USA), CLUSTALW (for example, version 1.83; Thompson et al., Nucleic Acids Research, 22(22):4673-4680 (1994)), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.), Vector NTI (Informax, Bethesda, Md.) and Sequencher v. 4.05. Within the context of this application it will be understood that where sequence analysis software is used for analysis, that the results of the analysis will be based on the “default values” of the program referenced, unless otherwise specified. As used herein “default values” will mean any set of values or parameters set by the software manufacturer that originally load with the software when first initialized.

### Structural and Functional Properties of the Soluble α-Glucan Fiber Composition Disclosed Herein

Human gastrointestinal enzymes readily recognize and digest linear α-glucan oligomers having a substantial amount of α-(1,4) glycosidic bonds. Replacing these linkages with alternative linkages such as α-(1,2); α-(1,3); and α-(1,6) typically reduces the digestibility of the α-glucan oligomers. Increasing the degree of branching (using alternative linkages) may also reduce the relative level of digestibility.

The present soluble α-glucan fiber composition was prepared from cane sugar (sucrose) using one or more enzymatic processing aids that have essentially the same amino acid sequences as found in nature (or active truncations thereof) from microorganisms which having a long history of exposure to humans (microorganisms naturally found in the oral cavity or found in foods such a beer, fermented soybeans, etc.). The soluble fibers have slow to no digestibility, exhibit high tolerance (i.e., as measured by an acceptable amount of gas formation), low viscosity (enabling use in a broad range of food applications), and are at least partially fermentable by gut microflora, providing possible prebiotic effects (for example, increasing the number and/or activity of bifidobacteria and lactic acid bacteria reported to be associated with providing potential prebiotic effects).

The soluble α-glucan fiber composition disclosed herein is characterized by the following combination of parameters:

a. at least 75% α-(1,3) glycosidic linkages;

b. less than 25% α-(1,6) glycosidic linkages;

c. less than 10% α-(1,3,6) glycosidic linkages;

d. a weight average molecular weight (Mw) of less than 5000 Daltons;

e. a viscosity of less than 0.25 Pascal second (Pa·s) at 12 wt % in water 20° C.;

f. a dextrose equivalence (DE) in the range of 4 to 40; and

g. a digestibility of less than 12% as measured by the Association of Analytical Communities (AOAC) method 2009.01;

h. a solubility of at least 20% (w/w) in pH 7 water at 25° C.; and

i. a polydispersity index (PDI) of less than 5.

The soluble α-glucan fiber composition disclosed herein comprises at least 75%, preferably at least 80%, more preferably at least 85%, even more preferably at least 90%, and most preferably at least 95% α-(1,3) glycosidic linkages.

In certain embodiments, in addition to the α-(1,3) glycosidic linkage embodiments described above, the soluble α-glucan fiber composition further comprises less than 25%, preferably less than 10%, more preferably 5% or less, and even more preferably less than 1% α-(1,6) glycosidic linkages.

In certain embodiments, in addition to the α-(1,3) and α-(1,6) glycosidic linkage content described above, the soluble α-glucan fiber composition further comprises less than 10%, preferably less than 5%, and most preferably less than 2.5% α-(1,3,6) glycosidic linkages.

In a preferred embodiment, the soluble α-glucan fiber composition comprises 93 to 97% α-(1,3) glycosidic linkages and less than 3% α-(1,6) glycosidic linkages and has a weight-average molecular weight corresponding to a DP of 3 to 7 mixture. In a further preferred embodiment, the soluble α-glucan fiber composition comprises about 95% α-(1,3) glycosidic linkages and about 1% α-(1,6) glycosidic linkages and has a weight-average molecular weight corresponding to a DP of 3 to 7 mixture. In certain further embodiments, the soluble α-glucan fiber composition further comprises 1 to 3% α-(1,3,6) linkages; preferably about 2% α-(1,3,6) linkages.

In certain emodiments, in addition to the above mentioned α-(1,3), α-(1,6), and/or α-(1,3,6) glycosidic linkage amounts, the soluble α-glucan fiber composition further comprises less than 5%, preferably less than 1%, and most preferably less than 0.5% α-(1,4) glycosidic linkages.

In another embodiment, in addition to the above mentioned glycosidic linkage amounts, the α-glucan fiber composition comprises a weight average molecular weight (M_{w}) of less than 5000 Daltons, preferably less than 2500 Daltons, more preferably between 500 and 2500 Daltons, and most preferably about 500 to about 2000 Daltons.

In another embodiment, in addition to any combination of the above features, the α-glucan fiber composition comprises a viscosity of less than 250 centipoise (0.25 Pascal second (Pa·s), preferably less than 10 centipoise (cP) (0.01 Pascal second (Pa·s)), preferably less than 7 cP (0.007 Pa·s), more preferably less than 5 cP (0.005 Pa·s), more preferably less than 4 cP (0.004 Pa·s), and most preferably less than 3 cP (0.003 Pa·s) at 12 wt % in water at 20° C.

The soluble α-glucan composition has a digestibility of less than 10%, preferably less than 9%, 8%, 7%, 6%, 5%, 4%, 3%, 2% or 1% digestible as measured by the Association of Analytical Communities (AOAC) method 2009.01. In another aspect, the relative level of digestibility may be alternatively determined using AOAC 2011.25 (Integrated Total Dietary Fiber Assay) (McCleary et al., (2012) J. AOAC Int., 95 (3), 824-844.

In addition to any of the above embodiments, in certain embodiments, the soluble α-glucan fiber composition has a solubility of at least 20% (w/w), preferably at least 30%, 40%, 50%, 60% or 70% in pH 7 water at 25° C.

In certain embodiments, the soluble α-glucan fiber composition comprises a reducing sugar content of less than 10 wt %, preferably less than 5 wt %, and most preferably 1 wt % or less.

In certain embodiments, the soluble α-glucan fiber composition comprises a caloric content of less than 4 kcal/g, preferably less than 3 kcal/g, more preferably less than 2.5 kcal/g, and most preferably about 2 kcal/g or less.

### Compositions Comprising Glucan Fibers

Depending upon the desired application, the soluble α-glucan fibers/fiber composition may be formulated (e.g., blended, mixed, incorporated into, etc.) with one or more other materials suitable for use in foods, personal care products and/or pharmaceuticals. As such, the present disclosure includes compositions comprising the soluble α-glucan fiber composition. The term “compositions comprising the soluble α-glucan fiber composition” in this context may include, for example, a nutritional or food composition, such as food products, food supplements, dietary supplements (for example, in the form of powders, liquids, gels, capsules, sachets or tables) or functional foods. In certain embodiments, “compositions comprising the soluble α-glucan fiber composition” includes personal care products, cosmetics, and pharmaceuticals.

The present soluble α-glucan fibers/fiber composition may be directly included as an ingredient in a desired product (e.g., foods, personal care products, etc.) or may be blended with one or more additional food grade materials to form a carbohydrate composition that is used in the desired product (e.g., foods, personal care products, etc.). The amount of the soluble α-glucan fiber composition incorporated into the carbohydrate composition may vary according to the application. As such, the present invention comprises a carbohydrate composition comprising the soluble α-glucan fiber composition. In certain embodiments, the carbohydrate composition comprises 0.01 to 99 wt % (dry solids basis), preferably 0.1 to 90 wt %, more preferably 1 to 90%, and most preferably 5 to 80 wt % of the soluble α-glucan fiber composition described above.

The term “food” as used herein is intended to encompass food for human consumption as well as for animal consumption. By “functional food” it is meant any fresh or processed food claimed to have a health-promoting and/or disease-preventing and/or disease-(risk)-reducing property beyond the basic nutritional function of supplying nutrients. Functional food may include, for example, processed food or foods fortified with health-promoting additives. Examples of functional food are foods fortified with vitamins, or fermented foods with live cultures.

A carbohydrate composition comprising the soluble α-glucan fiber composition may contain other materials known in the art for inclusion in nutritional compositions, such as water or other aqueous solutions, fats, sugars, starch, binders, thickeners, colorants, flavorants, odorants, acidulants (such as lactic acid or malic acid, among others), stabilizers, or high intensity sweeteners, or minerals, among others.

Examples of suitable food products include bread, breakfast cereals, biscuits, cakes, cookies, crackers, yogurt, kefir, miso, natto, tempeh, kimchee, sauerkraut, water, milk, fruit juice, vegetable juice, carbonated soft drinks, non-carbonated soft drinks, coffee, tea, beer, wine, liquor, alcoholic drink, snacks, soups, frozen desserts, fried foods, pizza, pasta products, potato products, rice products, corn products, wheat products, dairy products, hard candies, nutritional bars, cereals, dough, processed meats and cheeses, yoghurts, ice cream confections, milk-based drinks, salad dressings, sauces, toppings, desserts, confectionery products, cereal-based snack bars, prepared dishes, and the like. The carbohydrate composition comprising the present α-glucan fiber may be in the form of a liquid, powder, tablet, cube, granule, gel, or syrup.

In certain embodiments, the carbohydrate composition according to the invention comprises at least two fiber sources (i.e., at least one additional fiber source beyond the soluble α-glucan fiber composition). In certain embodiments, one fiber source is the soluble α-glucan fiber and the second fiber source is an oligo- or polysaccharide, selected from the group consisting of resistant/branched maltodextrins/fiber dextrins (such as NUTRIOSE® from Roquette Freres, Lestrem, France; FIBERSOL-2® from ADM-Matsutani LLC, Decatur, Ill.), polydextrose (LITESSE® from Danisco—DuPont Nutrition & Health, Wilmington, Del.), soluble corn fiber (for example, PROMITOR® from Tate & Lyle, London, UK), isomaltooligosaccharides (IMOs), alternan and/or maltoalternan oligosaccharides (MAOs) (for example, FIBERMALT™ from Aevotis GmbH, Potsdam, Germany; SUCROMALT™ (from Cargill Inc., Minneapolis, Minn.), pullulan, resistant starch, inulin, fructooligosaccharides (FOS), galactooligosaccharides (GOS), xylooligosaccharides, arabinoxylooligosaccharides, nigerooligosaccharides, gentiooligosaccharides, hemicellulose and fructose oligomer syrup.

The soluble α-glucan fiber can be added to foods as a replacement or supplement for conventional carbohydrates. As such, in certain embodiments, the invention is a food product comprising the soluble α-glucan fiber. In certain embodiments, the soluble α-glucan fiber composition in the food product is produced by a process disclosed herein.

The soluble α-glucan fiber composition may be used in a carbohydrate composition and/or food product comprising one or more high intensity artificial sweeteners including, but not limited to stevia, aspartame, sucralose, neotame, acesulfame potassium, saccharin, and combinations thereof. The soluble α-glucan fiber may be blended with sugar substitutes such as brazzein, curculin, erythritol, glycerol, glycyrrhizin, hydrogenated starch hydrolysates, inulin, isomalt, lactitol, mabinlin, maltitol, maltooligosaccharide, maltoalternan oligosaccharides (such as XTEND® SUCROMALT™, available from Cargill Inc., Minneapolis, Minn.), mannitol, miraculin, a mogroside mix, monatin, monellin, osladin, pentadin, sorbitol, stevia, tagatose, thaumatin, xylitol, and any combination thereof.

In certain embodiments, a food product containing the soluble α-glucan fiber composition will have a lower glycemic response, lower glycemic index, and lower glycemic load than a similar food product in which a conventional carbohydrate is used. Further, because the soluble α-glucan fiber is characterized by very low to no digestibility in the human stomach or small intestine, in certain embodiments, the caloric content of the food product is reduced. The soluble α-glucan fiber may be used in the form of a powder, blended into a dry powder with other suitable food ingredients or may be blended or used in the form of a liquid syrup comprising the dietary fiber (also referred to herein as an “soluble fiber syrup”, “fiber syrup” or simply the “syrup”). The “syrup” can be added to food products as a source of soluble fiber. It can increase the fiber content of food products without having a negative impact on flavor, mouth feel, or texture.

The fiber syrup can be used in food products alone or in combination with bulking agents, such as sugar alcohols or maltodextrins, to reduce caloric content and/or to enhance nutritional profile of the product. The fiber syrup can also be used as a partial replacement for fat in food products.

The fiber syrup can be used in food products as a tenderizer or texturizer, to increase crispness or snap, to improve eye appeal, and/or to improve the rheology of dough, batter, or other food compositions. The fiber syrup can also be used in food products as a humectant, to increase product shelf life, and/or to produce a softer, moister texture. It can also be used in food products to reduce water activity or to immobilize and manage water. Additional uses of the fiber syrup may include: replacement of an egg wash and/or to enhance the surface sheen of a food product, to alter flour starch gelatinization temperature, to modify the texture of the product, and to enhance browning of the product.

The fiber syrup can be used in a variety of types of food products. One type of food product in which the present syrup can be very useful is bakery products (i.e., baked foods), such as cakes, brownies, cookies, cookie crisps, muffins, breads, and sweet doughs. Conventional bakery products can be relatively high in sugar and high in total carbohydrates. The use of the fiber syrup as an ingredient in bakery products can help lower the sugar and carbohydrate levels, as well as reduce the total calories, while increasing the fiber content of the bakery product.

There are two main categories of bakery products: yeast-raised and chemically-leavened. In yeast-raised products, like donuts, sweet doughs, and breads, the fiber-containing syrup can be used to replace sugars, but a small amount of sugar may still be desired due to the need for a fermentation substrate for the yeast or for crust browning. The fiber syrup can be added with other liquids as a direct replacement for non-fiber containing syrups or liquid sweeteners. The dough would then be processed under conditions commonly used in the baking industry including being mixed, fermented, divided, formed or extruded into loaves or shapes, proofed, and baked or fried. The product can be baked or fried using conditions similar to traditional products. Breads are commonly baked at temperatures ranging from 420° F. to 520° F. (216-271° C.). for 20 to 23 minutes and doughnuts can be fried at temperatures ranging from 400-415° F. (204-213° C.), although other temperatures and times could also be used.

Chemically leavened products typically have more sugar and may contain have a higher level of the carbohydrate compositions and/or edible syrups comprising the soluble α-glucan fiber. A finished cookie can contain 30% sugar, which could be replaced, entirely or partially, with carbohydrate compositions and/or syrups comprising the present glucan fiber composition. These products could have a pH of 4-9.5, for example. The moisture content can be between 2-40%, for example.

The carbohydrate compositions and/or fiber-containing syrups are readily incorporated and may be added to the fat at the beginning of mixing during a creaming step or in any method similar to the syrup or dry sweetener that it is being used to replace. The product would be mixed and then formed, for example by being sheeted, rotary cut, wire cut, or through another forming process. The products would then be baked under typical baking conditions, for example at 200-450° F. (93-232° C.).

Another type of food product in which the carbohydrate compositions and/or fiber-containing syrups can be used is breakfast cereal. For example, fiber-containing syrups could be used to replace all or part of the sugar in extruded cereal pieces and/or in the coating on the outside of those pieces. The coating is typically 30-60% of the total weight of the finished cereal piece. The syrup can be applied in a spray or drizzled on, for example.

Another type of food product in which the soluble α-glucan fiber composition (optionally used in the form of a carbohydrate composition and/or fiber-containing syrup) can be used is dairy products. Examples of dairy products in which it can be used include yogurt, yogurt drinks, milk drinks, flavored milks, smoothies, ice cream, shakes, cottage cheese, cottage cheese dressing, and dairy desserts, such as quarg and the whipped mousse-type products. This would include dairy products that are intended to be consumed directly (such as packaged smoothies) as well as those that are intended to be blended with other ingredients (such as blended smoothies). It can be used in pasteurized dairy products, such as ones that are pasteurized at a temperature from 160° F. to 285° F. (71-141° C.).

Another type of food product in which the composition comprising the soluble α-glucan fiber composition can be used is confections. Examples of confections in which it can be used include hard candies, fondants, nougats and marshmallows, gelatin jelly candies or gummies, jellies, chocolate, licorice, chewing gum, caramels and toffees, chews, mints, tableted confections, and fruit snacks. In fruit snacks, a composition comprising the soluble α-glucan fiber could be used in combination with fruit juice. The fruit juice would provide the majority of the sweetness, and the composition comprising the soluble α-glucan fiber would reduce the total sugar content and add fiber. Compositions comprising the soluble α-glucan fiber can be added to the initial candy slurry and heated to the finished solids content. The slurry could be heated from 200-305° F. (93-152° C.) to achieve the finished solids content. Acid could be added before or after heating to give a finished pH of 2-7. The composition comprising the glucan fiber could be used as a replacement for 0-100% of the sugar and 1-100% of the corn syrup or other sweeteners present.

Another type of food product in which a composition comprising the soluble α-glucan fiber composition can be used is jams and jellies. Jams and jellies are made from fruit. A jam contains fruit pieces, while jelly is made from fruit juice. The composition comprising the present fiber can be used in place of sugar or other sweeteners as follows: weigh fruit and juice into a tank; premix sugar, the soluble α-glucan fiber-containing composition and pectin; add the dry composition to the liquid and cook to a temperature of 214-220° F. (101-104° C.); hot fill into jars and retort for 5-30 minutes.

Another type of food product in which a composition comprising the soluble α-glucan fiber composition (such as a fiber-containing syrup) can be used is beverages. Examples of beverages in which it can be used include carbonated beverages, fruit juices, concentrated juice mixes (e.g., margarita mix), clear waters, and beverage dry mixes. The use of the soluble α-glucan fiber may overcome the clarity problems that result when other types of fiber are added to beverages. A complete replacement of sugars may be possible (which could be, for example, being up to 12% or more of the total formula).

Another type of food product is high solids fillings. Examples of high solids fillings include fillings in snack bars, toaster pastries, donuts, and cookies. The high solids filling could be an acid/fruit filling or a savory filling, for example. The soluble α-glucan fiber composition could be added to products that would be consumed as is, or products that would undergo further processing, by a food processor (additional baking) or by a consumer (bake stable filling). In certain embodiments, the high solids fillings would have a solids concentration between 67-90%. The solids could be entirely replaced with a composition comprising the soluble α-glucan fiber or it could be used for a partial replacement of the other sweetener solids present (e.g., replacement of current solids from 5-100%). Typically fruit fillings would have a pH of 2-6, while savory fillings would be between 4-8 pH. Fillings could be prepared cold or heated at up to 250° F. (121° C.) to evaporate to the desired finished solids content.

Another type of food product in which the soluble α-glucan fiber composition or a carbohydrate composition (comprising the α-glucan fiber composition) can be used is extruded and sheeted snacks. Examples of extruded and sheeted can be used include puffed snacks, crackers, tortilla chips, and corn chips. In preparing an extruded piece, a composition comprising the present glucan fiber would be added directly with the dry products. A small amount of water would be added in the extruder, and then it would pass through various zones ranging from 100° F. to 300° F. (38-149° C.). The dried product could be added at levels from 0-50% of the dry products mixture. A syrup comprising the soluble α-glucan fiber could also be added at one of the liquid ports along the extruder. The product would come out at either a low moisture content (5%) and then baked to remove the excess moisture, or at a slightly higher moisture content (10%) and then fried to remove moisture and cook out the product. Baking could be at temperatures up to 500° F. (260° C.). for 20 minutes. Baking would more typically be at 350° F. (177° C.) for 10 minutes. Frying would typically be at 350° F. (177° C.) for 2-5 minutes. In a sheeted snack, the composition comprising the soluble α-glucan fiber could be used as a partial replacement of the other dry ingredients (for example, flour). The soluble α-glucan fiber could be from 0-50% of the dry weight. The product would be dry mixed, and then water added to form cohesive dough. The product mix could have a pH from 5 to 8. The dough would then be sheeted and cut and then baked or fried. Baking could be at temperatures up to 500° F. (260° C.) for 20 minutes. Frying would typically be at 350° F. (177° C.) for 2-5 minutes. Another potential benefit from the use of a composition comprising the soluble α-glucan fiber is a reduction of the fat content of fried snacks by as much as 15% when it is added as an internal ingredient or as a coating on the outside of a fried food.

Another type of food product in which a fiber-containing syrup can be used is gelatin desserts. The ingredients for gelatin desserts are often sold as a dry mix with gelatin as a gelling agent. The sugar solids could be replaced partially or entirely with a composition comprising the present glucan fiber in the dry mix. The dry mix can then be mixed with water and heated to 212° F. (100° C.). to dissolve the gelatin and then more water and/or fruit can be added to complete the gelatin dessert. The gelatin is then allowed to cool and set. Gelatin can also be sold in shelf stable packs. In that case the stabilizer is usually carrageenan-based. As stated above, a composition comprising the soluble α-glucan fiber could be used to replace up to 100% of the other sweetener solids. The dry ingredients are mixed into the liquids and then pasteurized and put into cups and allowed to cool and set.

Another type of food product in which a composition comprising the soluble α-glucan fiber can be used is snack bars. Examples of snack bars in which it can be used include breakfast and meal replacement bars, nutrition bars, granola bars, protein bars, and cereal bars. It could be used in any part of the snack bars, such as in the high solids filling, the binding syrup or the particulate portion. A complete or partial replacement of sugar in the binding syrup may be possible. The binding syrup is typically from 50-90% solids and applied at a ratio ranging from 10% binding syrup to 90% particulates, to 70% binding syrup to 30% particulates. The binding syrup is made by heating a solution of sweeteners, bulking agents and other binders (like starch) to 160-230° F. (71-110° C.) (depending on the finished solids needed in the syrup). The syrup is then mixed with the particulates to coat the particulates, providing a coating throughout the matrix. A composition comprising the soluble α-glucan fiber could also be used in the particulates themselves. This could be an extruded piece, directly expanded or gun puffed. It could be used in combination with another grain ingredient, corn meal, rice flour or other similar ingredient.

Another type of food product in which a composition comprising the soluble α-glucan fiber syrup can be used is cheese, cheese sauces, and other cheese products. Examples of cheese, cheese sauces, and other cheese products in which it can be used include lower milk solids cheese, lower fat cheese, and calorie reduced cheese. In block cheese, it can help to improve the melting characteristics, or to decrease the effect of the melt limitation added by other ingredients such as starch. It could also be used in cheese sauces, for example as a bulking agent, to replace fat, milk solids, or other typical bulking agents.

Another type of food product in which a composition comprising the soluble α-glucan fiber can be used is films that are edible and/or water soluble. Examples of films in which it can be used include films that are used to enclose dry mixes for a variety of foods and beverages that are intended to be dissolved in water, or films that are used to deliver color or flavors such as a spice film that is added to a food after cooking while still hot. Other film applications include, but are not limited to, fruit and vegetable leathers, and other flexible films.

In another embodiment, compositions comprising the soluble α-glucan fiber can be used is soups, syrups, sauces, and dressings. A typical dressing could be from 0-50% oil, with a pH range of 2-7. It could be cold processed or heat processed. It would be mixed, and then stabilizer would be added. The composition comprising the soluble α-glucan fiber could easily be added in liquid or dry form with the other ingredients as needed. The dressing composition may need to be heated to activate the stabilizer. Typical heating conditions would be from 170-200° F. (77-93° C.) for 1-30 minutes. After cooling, the oil is added to make a pre-emulsion. The product is then emulsified using a homogenizer, colloid mill, or other high shear process.

Sauces can have from 0-10% oil and from 10-50% total solids, and can have a pH from 2-8. Sauces can be cold processed or heat processed. The ingredients are mixed and then heat processed. The composition comprising the soluble α-glucan fiber could easily be added in liquid or dry form with the other ingredients as needed. Typical heating would be from 170-200° F. (77-93° C.) for 1-30 minutes.

Soups are more typically 20-50% solids and in a more neutral pH range (4-8). They can be a dry mix, to which a dry composition comprising the soluble α-glucan fiber could be added, or a liquid soup which is canned and then retorted. In soups, resistant corn syrup could be used up to 50% solids, though a more typical usage would be to deliver 5 g of fiber/serving.

Another type of food product in which a composition comprising the soluble α-glucan fiber composition can be used is coffee creamers. Examples of coffee creamers in which it can be used include both liquid and dry creamers. A dry blended coffee creamer can be blended with commercial creamer powders of the following fat types: soybean, coconut, palm, sunflower, or canola oil, or butterfat. These fats can be non-hydrogenated or hydrogenated. The composition comprising the soluble α-glucan fiber composition can be added as a fiber source, optionally together with fructo-oligosaccharides, polydextrose, inulin, maltodextrin, resistant starch, sucrose, and/or conventional corn syrup solids. The composition can also contain high intensity sweeteners, such as sucralose, acesulfame potassium, aspartame, or combinations thereof. These ingredients can be dry blended to produce the desired composition.

A spray dried creamer powder is a combination of fat, protein and carbohydrates, emulsifiers, emulsifying salts, sweeteners, and anti-caking agents. The fat source can be one or more of soybean, coconut, palm, sunflower, or canola oil, or butterfat. The protein can be sodium or calcium caseinates, milk proteins, whey proteins, wheat proteins, or soy proteins. The carbohydrate could be a composition comprising the present α-glucan fiber composition alone or in combination with fructooligosaccharides, polydextrose, inulin, resistant starch, maltodextrin, sucrose, corn syrup or any combination thereof. The emulsifiers can be mono- and diglycerides, acetylated mono- and diglycerides, or propylene glycol monoesters. The salts can be trisodium citrate, monosodium phosphate, disodium phosphate, trisodium phosphate, tetrasodium pyrophosphate, monopotassium phosphate, and/or dipotassium phosphate. The composition can also contain high intensity sweeteners, such as those describe above. Suitable anti-caking agents include sodium silicoaluminates or silica dioxides. The products are combined in slurry, optionally homogenized, and spray dried in either a granular or agglomerated form.

Liquid coffee creamers are simply a homogenized and pasteurized emulsion of fat (either dairy fat or hydrogenated vegetable oil), some milk solids or caseinates, corn syrup, and vanilla or other flavors, as well as a stabilizing blend. The product is usually pasteurized via HTST (high temperature short time) at 185° F. (85° C.) for 30 seconds, or UHT (ultra-high temperature), at 285° F. (141° C.) for 4 seconds, and homogenized in a two stage homogenizer at 500-3000 psi (3.45-20.7 MPa) first stage, and 200-1000 psi (1.38-6.89 MPa) second stage. The coffee creamer is usually stabilized so that it does not break down when added to the coffee.

Another type of food product in which a composition comprising the soluble α-glucan fiber composition (such as a fiber-containing syrup) can be used is food coatings such as icings, frostings, and glazes. In icings and frostings, the fiber-containing syrup can be used as a sweetener replacement (complete or partial) to lower caloric content and increase fiber content. Glazes are typically about 70-90% sugar, with most of the rest being water, and the fiber-containing syrup can be used to entirely or partially replace the sugar. Frosting typically contains about 2-40% of a liquid/solid fat combination, about 20-75% sweetener solids, color, flavor, and water. The fiber-containing syrup can be used to replace all or part of the sweetener solids, or as a bulking agent in lower fat systems.

Another type of food product in which the fiber-containing syrup can be used is pet food, such as dry or moist dog food. Pet foods are made in a variety of ways, such as extrusion, forming, and formulating as gravies. The fiber-containing syrup could be used at levels of 0-50% in each of these types.

Another type of food product in which a composition comprising the soluble α-glucan fiber composition, such as a syrup, can be used is fish and meat. Conventional corn syrup is already used in some meats, so a fiber-containing syrup can be used as a partial or complete substitute. For example, the syrup could be added to brine before it is vacuum tumbled or injected into the meat. It could be added with salt and phosphates, and optionally with water binding ingredients such as starch, carrageenan, or soy proteins. This would be used to add fiber, a typical level would be 5 g/serving which would allow a claim of excellent source of fiber.

Personal Care and/or Pharmaceutical Compositions Comprising the Present Soluble Fiber

The soluble α-glucan fiber and/or compositions comprising the soluble α-glucan fiber may be used in personal care products. For example, one may be able to use such materials as a humectants, hydrocolloids or possibly thickening agents. The present fibers and/or compositions comprising the present fibers may be used in conjunction with one or more other types of thickening agents if desired, such as those disclosed in U.S. Pat. No. 8,541,041, the disclosure of which is incorporated herein by reference in its entirety.

Personal care products herein include, but are not limited to, for example, skin care compositions, cosmetic compositions, antifungal compositions, and antibacterial compositions. Personal care products herein may be in the form of, for example, lotions, creams, pastes, balms, ointments, pomades, gels, liquids, combinations of these and the like. The personal care products disclosed herein can include at least one active ingredient. An active ingredient is generally recognized as an ingredient that produces an intended pharmacological effect.

In certain embodiments, a skin care product can be applied to skin for addressing skin damage related to a lack of moisture. A skin care product may also be used to address the visual appearance of skin (e.g., reduce the appearance of flaky, cracked, and/or red skin) and/or the tactile feel of the skin (e.g., reduce roughness and/or dryness of the skin while improved the softness and subtleness of the skin). A skin care product typically may include at least one active ingredient for the treatment or prevention of skin ailments, providing a cosmetic effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these. A skin care product may include one or more natural moisturizing factors such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example. Other ingredients that may be included in a skin care product include, without limitation, glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil.

A personal care product, as used herein, can also be in the form of makeup or other product including, but not limited to, a lipstick, mascara, rouge, foundation, blush, eyeliner, lip liner, lip gloss, other cosmetics, sunscreen, sun block, nail polish, mousse, hair spray, styling gel, nail conditioner, bath gel, shower gel, body wash, face wash, shampoo, hair conditioner (leave-in or rinse-out), cream rinse, hair dye, hair coloring product, hair shine product, hair serum, hair anti-frizz product, hair split-end repair product, lip balm, skin conditioner, cold cream, moisturizer, body spray, soap, body scrub, exfoliant, astringent, scruffing lotion, depilatory, permanent waving solution, antidandruff formulation, antiperspirant composition, deodorant, shaving product, pre-shaving product, after-shaving product, cleanser, skin gel, rinse, toothpaste, or mouthwash, for example.

A pharmaceutical product, as used herein, can be in the form of an emulsion, liquid, elixir, gel, suspension, solution, cream, capsule, tablet, sachet or ointment, for example. Also, a pharmaceutical product herein can be in the form of any of the personal care products disclosed herein. A pharmaceutical product can further comprise one or more pharmaceutically acceptable carriers, diluents, and/or pharmaceutically acceptable salts. The present fibers and/or compositions comprising the present fibers can also be used in capsules, encapsulants, tablet coatings, and as an excipients for medicaments and drugs.

### Enzymatic Synthesis of the Soluble α-Glucan Fiber Composition

Methods are provided to enzymatically produce a soluble α-glucan fiber composition. In an embodiment, the method comprises the use of at least one recombinantly produced glucosyltransferase belonging to the glucoside hydrolase type 70 family (E.C. 2.4.1.-), and which is capable of catalyzing the synthesis of a digestion resistant soluble α-glucan fiber composition using sucrose as a substrate. Glycoside hydrolase family 70 enzymes are transglucosidases produced by lactic acid bacteria such as Streptococcus, Leuconostoc, Weisella or Lactobacillus genera (see Carbohydrate Active Enzymes database; “CAZy”; Cantarel et al., (2009) Nucleic Acids Res 37:D233-238). The recombinantly expressed glucosyltransferase(s) preferably have an amino acid sequence identical to that found in nature (i.e., the same as the full length sequence as found in the source organism or a catalytically active truncation thereof).

GTF enzymes are able to polymerize the D-glucosyl units of sucrose to form homooligosaccharides or homopolysaccharides. Depending upon the specificity of the GTF enzyme, linear and/or branched glucans comprising various glycosidic linkages are formed such as α-(1,2), α-(1,3), α-(1,4) and α-(1,6). Glucosyltransferases may also transfer the D-glucosyl units onto hydroxyl acceptor groups. A non-limiting list of acceptors include carbohydrates, alcohols, polyols or flavonoids. The structure of the resultant glucosylated product is dependent upon the enzyme specificity.

In the present invention, the D-glucopyranosyl donor is sucrose. As such the reaction is:

Sucrose+GTF [symbol] α-D-(Glucose)ₙ+D-Fructose+GTF

The type of glycosidic linkage predominantly formed is used to name/classify the glucosyltransferase enzyme. Examples include dextransucrases (α-(1,6) linkages; EC 2.4.1.5), mutansucrases (α-(1,3) linkages; EC 2.4.1.-), alternansucrases (alternating α(1,3)-α(1,6) backbone; EC 2.4.1.140), and reuteransucrases (mix of α-(1,4) and α-(1,6) linkages; EC 2.4.1.-).

In one aspect, the glucosyltransferase (GTF) is capable of forming glucans having 50% or more α-(1,3) glycosidic linkages with the proviso that the glucan product is not an alternan (i.e., the enzyme is not an alternansucrase). In a preferred aspect, the glucosyltransferase is a mutansucrase (EC 2.4.1.-). As described above, amino acid residues which influence mutansucrase function have previously been characterized. See, A. Shimamura et al. (J. Bacteriology, (1994) 176:4845-4850).

The glucosyltransferase is preferably a glucosyltransferase capable of producing a glucan with at least 75% α-(1,3) glycosidic linkages. In certain embodiments, the glucosyltransferase comprises an amino acid sequence having at least 90% sequence identity, including at least 95%, at least 96%, at least 97%, at least 98%, at least 99%, or which is identical to SEQ ID NO: 153. In certain embodiments, the glucosyltransferase comprising an amino acid sequence with 90% or greater sequence identity to SEQ ID NO: 153 is GTF-S, a homolog thereof, a truncation thereof, or a truncation of a homolog thereof. In certain embodiments, the glucosyltransferase comprises an amino acid sequence selected from the group consisting of SEQ ID NOs: 3, 5, 17, 19, 88, 90, 92, 94, 96, 98, 100, 102, 104, 106, 108, 110, 112, and any combination thereof. However, it should be noted that some wild type sequences may be found in nature in a truncated form. As such, and in a further embodiment, the glucosyltransferase suitable for use may be a truncated form of the wild type sequence. In a further embodiment, the truncated glucosyltransferase comprises a sequence derived from the full length wild type amino acid sequence selected from the group consisting of SEQ ID NOs: 3 and 17. In another embodiment, the glucosyltransferase may be truncated and will have an amino acid sequence selected from the group consisting of SEQ ID NOs: 5 and 19. In another embodiment, the glucosyltransferase comprises SEQ ID NO: 5. In yet another embodiment, the glucosyltransferase is truncated and is derived from SEQ ID NO: 19. In certain other embodiments, the truncated glucosyltransferase comprises an amino acid sequence selected from the group consisting of SEQ ID NOs: 118, 120, 122, 124, 126, 128, 130, 132, 134, 136, 138, 140, 142, 144, 146, 148, 150, and 152.

The concentration of the catalyst in the aqueous reaction formulation depends on the specific catalytic activity of the catalyst, and is chosen to obtain the desired rate of reaction. The weight of each catalyst (either a single glucosyltransferase or individually a glucosyltransferase and α-glucanohydrolase) reactions typically ranges from 0.0001 mg to 20 mg per mL of total reaction volume, preferably from 0.001 mg to 10 mg per mL. The catalyst may also be immobilized on a soluble or insoluble support using methods well-known to those skilled in the art; see for example, Immobilization of Enzymes and Cells; Gordon F. Bickerstaff, Editor; Humana Press, Totowa, N.J., USA; 1997. The use of immobilized catalysts permits the recovery and reuse of the catalyst in subsequent reactions. The enzyme catalyst may be in the form of whole microbial cells, permeabilized microbial cells, microbial cell extracts, partially-purified or purified enzymes, and mixtures thereof.

The pH of the final reaction formulation is from about 3 to about 8, preferably from about 4 to about 8, more preferably from about 5 to about 8, even more preferably about 5.5 to about 7.5, and yet even more preferably about 5.5 to about 6.5. The pH of the reaction may optionally be controlled by the addition of a suitable buffer including, but not limited to, phosphate, pyrophosphate, bicarbonate, acetate, or citrate. The concentration of buffer, when employed, is typically from 0.1 mM to 1.0 M, preferably from 1 mM to 300 mM, most preferably from 10 mM to 100 mM.

The sucrose concentration initially present when the reaction components are combined is at least 50 g/L, preferably 50 g/L to 600 g/L, more preferably 100 g/L to 500 g/L, more preferably 150 g/L to 450 g/L, and most preferably 250 g/L to 450 g/L. The substrate for the α-glucanohydrolase (when present) will be the members of the glucose oligomer population formed by the glucosyltransferase. As the glucose oligomers present in the reaction system may act as acceptors, the exact concentration of each species present in the reaction system will vary. Additionally, other acceptors may be added (i.e., external acceptors) to the initial reaction mixture such as maltose, isomaltose, isomaltotriose, and methyl-α-D-glucan, to name a few.

The length of the reaction may vary and may often be determined by the amount of time it takes to use all of the available sucrose substrate. In one embodiment, the reaction is conducted until at least 90%, preferably at least 95% and most preferably at least 99% of the sucrose initially present in the reaction mixture is consumed. In another embodiment, the reaction time is 1 hour to 168 hours, preferably 1 hour to 72 hours, and most preferably 1 hour to 24 hours.

Single Enzyme Method (Glucosyltransferase) using Elevated Reaction Temperature

The optimum temperature for many GH70 family glucosyltransferases is often between 25° C. and 35° C. with rapid inactivation often observed at temperatures exceeding 55° C.-60° C. However, it has been discovered that certain glucosyltransferases may be capable of producing the desired soluble α-glucan fiber composition from sucrose when the reaction is conducted at elevated temperatures (defined herein as a temperature of at least 45° C. yet below the inactivation temperature of the enzyme under the reaction conditions employed).

In one aspect, the glucosyltransferase is capable of producing the soluble α-glucan fiber from sucrose when the reaction is conducted at a temperature of at least 45° C., but below the temperature where the enzyme is thermally inactivated under the reaction conditions employed. In a further aspect, the temperature for running the glucosyltransferase reaction is conducted at a temperature of at least 47° C. but less than the inactivation temperature of the specified enzyme under the reaction conditions employed. In one aspect, the upper limit of the reaction temperature is equal to or less than 55° C. In another embodiment, the reaction temperature is 47° C. to 52° C. In a further aspect, the glucosyltransferase used in the single enzyme method comprises an amino acid sequence derived from a polypeptide having an amino acid sequence selected from the group consisting of SEQ ID NO: 3 and 5. In a preferred aspect, the glucosyltransferase is derived from the Streptococcus salivarius GtfJ glucosyltransferase (GENBANK® gi: 47527; SEQ ID NO: 3). In a further preferred embodiment, the glucosyltransferase is SEQ ID NO: 3 or a catalytically active truncation retaining the glucosyltransferase activity thereof.

### Soluble Glucan Fiber Synthesis—Reaction Systems Comprising a Glucosyltransferase (Gtf) and an α-Glucanohydrolase

A method is provided to enzymatically produce the soluble α-glucan fibers using at least one α-glucanohydrolase in combination (i.e., concomitantly in the reaction mixture) with at least one of the above glucosyltransferases. The simultaneous use of the two enzymes produces a different product profile (i.e., the profile of the soluble fiber composition) when compared to a sequential application of the same enzymes (i.e., first synthesizing the glucan polymer from sucrose using a glucosyltransferase and then subsequently treating the glucan polymer with an α-glucanohydrolase). In one embodiment, a glucan fiber synthesis method based on sequential application of a glucosyltransferase with an α-glucanohydrolase is specifically excluded.

Similar to the glucosyltransferases, an α-glucanohydrolase may be defined by the endohydrolysis activity towards certain α-D-glycosidic linkages. α-glucanohydrolases useful in the methods disclosed herein can be identified by their characteristic domain structures, for example, those domain structures identified for mutanases and dextranases described above. Examples may include, but are not limited to, dextranases (capable of hydrolyzing α-(1,6)-linked glycosidic bonds; E.C. 3.2.1.11), mutanases (capable of hydrolyzing α-(1,3)-linked glycosidic bonds; E.C. 3.2.1.59), mycodextranases (capable of endohydrolysis of (1→4)-α-D-glucosidic linkages in α-D-glucans containing both (1→3)- and (1→4)-bonds; EC 3.2.1.61), glucan 1,6-α-glucosidase (EC 3.2.1.70), and alternanases (capable of endohydrolytically cleaving alternan; E.C. 3.2.1.-; see U.S. Pat. No. 5,786,196). Various factors including, but not limited to, level of branching, the type of branching, and the relative branch length within certain α-glucans may adversely impact the ability of an α-glucanohydrolase to endohydrolyze some glycosidic linkages.

In one embodiment, the α-glucanohydrolase is a dextranase (EC 3.2.1.11), a mutanase (EC 3.1.1.59) ora combination thereof. In one embodiment, the dextranase is a food grade dextranase from Chaetomium erraticum. In a further embodiment, the dextranase from Chaetomium erraticum is DEXTRANASE® PLUS L, available from Novozymes A/S, Denmark.

In another embodiment, the α-glucanohydrolase is at least one mutanase (EC 3.1.1.59). Mutanases useful in the methods disclosed herein can be identified by their characteristic structure. See, e.g., Y. Hakamada et al. (Biochimie, (2008) 90:525-533). In one embodiment, the mutanase is one obtainable from the genera Penicillium, Paenibacillus, Hypocrea, Aspergillus, and Trichoderma. In a further embodiment, the mutanase is from Penicillium marneffei ATCC 18224 or Paenibacillus Humicus. In one embodiment, the mutanase comprises an amino acid sequence selected from SEQ ID NOs 21, 22, 24, 27, 29, 54, 56, 58, and any combination thereof. In yet a further embodiment, the mutanase comprises an amino acid sequence selected from SEQ ID NO: 21, 22, 24, 27 and any combination thereof. In another embodiment, the above mutanases may be a catalytically active truncation so long as the mutanase activity is retained

The temperature of the enzymatic reaction system comprising concomitant use of at least one glucosyltransferase and at least one α-glucanohydrolase may be chosen to control both the reaction rate and the stability of the enzyme catalyst activity. The temperature of the reaction may range from just above the freezing point of the reaction formulation (approximately 0° C.) to about 60° C., with a preferred range of 5° C. to about 55° C., and a more preferred range of reaction temperature of from about 20° C. to about 47° C.

The ratio of glucosyltransferase to α-glucanohydrolase (v/v) may vary depending upon the selected enzymes. In one embodiment, the ratio of glucosyltransferase to α-glucanohydrolase (v/v) ranges from 1:0.01 to 0.01:1.0. In another embodiment, the ratio of glucosyltransferase to α-glucanohydrolase (units of activity/units of activity) may vary depending upon the selected enzymes. In still further embodiments, the ratio of glucosyltransferase to α-glucanohydrolase (units of activity/units of activity) ranges from 1:0.01 to 0.01:1.0.

In one embodiment, a method is provided to produce a soluble α-glucan fiber composition comprising:

1. providing a set of reaction components comprising:
a. sucrose;
b. at least one glucosyltransferase capable of catalyzing the synthesis of glucan polymers having at least 75% α-(1,3) glycosidic linkages;
c. at least one α-glucanohydrolase capable of hydrolyzing glucan polymers having one or more α-(1,3) glycosidic linkages or one or more α-(1,6) glycosidic linkages; and
d. optionally one more acceptors; and

2. combining the set of reaction components under suitable aqueous reaction conditions whereby a soluble α-glucan fiber composition is produced.

In a preferred embodiment, the at least one glucosyltransferase and the at least one α-glucanohydrolase are concomitantly present in the reaction to produce the soluble α-glucan fiber composition.

In one embodiment, the at least one glucosyltransferase capable of catalyzing the synthesis of glucan polymers having one or more α-(1,3) glycosidic linkages is a mutansucrase.

In another embodiment, the at least one α-glucanohydrolase capable of hydrolyzing glucan polymers having one or more α-(1,3) glycosidic linkages or one or more α-(1,6) glycosidic linkages is an endomutanase.

In a preferred embodiment, the set of reaction components comprises the concomitant use of a mutansucrase and a mutanase.

The method to produce a soluble α-glucan fiber may further comprise one or more additional steps to obtain the soluble α-glucan fiber composition. As such, and in a further embodiment, a method is provided comprising:
1. providing a set of reaction components comprising:
   a) sucrose;
   b) at least one glucosyltransferase capable of catalyzing the synthesis of glucan polymers having at least 75% α-(1,3) glycosidic linkages;
   c) at least one α-glucanohydrolase capable of hydrolyzing glucan polymers having one or more α-(1,3) glycosidic linkages or one or more α-(1,6) glycosidic linkages; and
   d) optionally one or more acceptors;
2. combining the set of reaction components under suitable aqueous reaction conditions whereby a product mixture comprising a soluble α-glucan fiber composition is produced;
3. isolating the soluble α-glucan fiber composition from the product mixture of step 2; and
4. optionally concentrating the soluble α-glucan fiber composition.

### Methods to Identify Substantially Similar Enzymes Having the Desired Activity

The skilled artisan recognizes that substantially similar enzyme sequences may also be used in the present compositions and methods so long as the desired activity is retained (i.e., glucosyltransferase activity capable of forming glucans having the desired glycosidic linkages or α-glucanohydrolases having endohydrolytic activity towards the target glycosidic linkage(s)). For example, it has been demonstrated that catalytically active truncations may be prepared and used so long as the desired activity is retained (or even improved in terms of specific activity). In one embodiment, substantially similar sequences are defined by their ability to hybridize, under highly stringent conditions with the nucleic acid molecules associated with sequences exemplified herein. In another embodiment, sequence alignment algorithms may be used to define substantially similar enzymes based on the percent identity to the DNA or amino acid sequences provided herein.

As used herein, a nucleic acid molecule is “hybridizable” to another nucleic acid molecule, such as a cDNA, genomic DNA, or RNA, when a single strand of the first molecule can anneal to the other molecule under appropriate conditions of temperature and solution ionic strength. Hybridization and washing conditions are well known and exemplified in Sambrook, J. and Russell, D., T. Molecular Cloning: A Laboratory Manual, Third Edition, Cold Spring Harbor Laboratory Press, Cold Spring Harbor (2001). The conditions of temperature and ionic strength determine the “stringency” of the hybridization. Stringency conditions can be adjusted to screen for moderately similar molecules, such as homologous sequences from distantly related organisms, to highly similar molecules, such as genes that duplicate functional enzymes from closely related organisms. Post-hybridization washes typically determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of conditions uses higher temperatures in which the washes are identical to those above except for the temperature of the final two 30 min washes in 0.2×SSC, 0.5% SDS was increased to 60° C. Another preferred set of highly stringent hybridization conditions is 0.1×SSC, 0.1% SDS, 65° C. and washed with 2×SSC, 0.1% SDS followed by a final wash of 0.1×SSC, 0.1% SDS, 65° C.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (Sambrook, J. and Russell, D., T., supra). For hybridizations with shorter nucleic acids, i.e., oligonucleotides, the position of mismatches becomes more important, and the length of the oligonucleotide determines its specificity. In one aspect, the length for a hybridizable nucleic acid is at least about 10 nucleotides. Preferably, a minimum length for a hybridizable nucleic acid is at least about 15 nucleotides in length, more preferably at least about 20 nucleotides in length, even more preferably at least 30 nucleotides in length, even more preferably at least 300 nucleotides in length, and most preferably at least 800 nucleotides in length. Furthermore, the skilled artisan will recognize that the temperature and wash solution salt concentration may be adjusted as necessary according to factors such as length of the probe.

As used herein, the term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the number of matching nucleotides or amino acids between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Methods to determine identity and similarity are codified in publicly available computer programs. Sequence alignments and percent identity calculations may be performed using the Megalign program of the LASERGENE bioinformatics computing suite (DNASTAR Inc., Madison, Wis.), the AlignX program of Vector NTI v. 7.0 (Informax, Inc., Bethesda, Md.), or the EMBOSS Open Software Suite (EMBL-EBI; Rice et al., Trends in Genetics 16, (6):276-277 (2000)). Multiple alignment of the sequences can be performed using the CLUSTAL method (such as CLUSTALW; for example version 1.83) of alignment (Higgins and Sharp, CABIOS, 5:151-153 (1989); Higgins et al., Nucleic Acids Res. 22:4673-4680 (1994); and Chenna et al., Nucleic Acids Res 31 (13):3497-500 (2003)), available from the European Molecular Biology Laboratory via the European Bioinformatics Institute) with the default parameters. Suitable parameters for CLUSTALW protein alignments include GAP Existence penalty=15, GAP extension=0.2, matrix=Gonnet (e.g., Gonnet250), protein ENDGAP=−1, protein GAPDIST=4, and KTUPLE=1. In one embodiment, a fast or slow alignment is used with the default settings where a slow alignment is preferred. Alternatively, the parameters using the CLUSTALW method (e.g., version 1.83) may be modified to also use KTUPLE=1, GAP PENALTY=10, GAP extension=1, matrix=BLOSUM (e.g., BLOSUM64), WINDOW=5, and TOP DIAGONALS SAVED=5.

In one aspect, suitable isolated nucleic acid molecules encode a polypeptide having an amino acid sequence that is at least about 20%, preferably at least 30%, 40%, 50%, 60%, 70%, 80%, 85%, 90%, 91%, 92%, 93%, 94%, 95%, 96%, 97%, 98%, or 99% identical to an amino acid sequence reported herein. In another aspect, suitable isolated nucleic acid molecules encode a polypeptide having an amino acid sequence that is at least about 20%, preferably at least 30%, 40%, 50%, 60%, 70%, 80%, 85%, 90%, 91%, 92%, 93%, 94%, 95%, 96%, 97%, 98%, or 99% identical to an amino acid sequence reported herein; with the proviso that the polypeptide retains the respective activity (i.e., glucosyltransferase or α-glucanohydrolase activity). In certain embodiments, glucosyltransferases which retain the activity include those glucosyltransfereases which comprise an amino acid sequence which is at least 90% identical to SEQ ID NO: 153.

### Gas Production

A rapid rate of gas production in the lower gastrointestinal tract gives rise to gastrointestinal discomfort such as flatulence and bloating, whereas if gas production is gradual and low, the body can more easily cope. For example, inulin gives a boost of gas production which is rapid and high when compared to the disclosed soluble α-glucan fiber composition at an equivalent dosage (grams soluble fiber), whereas the disclosed soluble α-glucan fiber composition preferably has a rate of gas release that is lower than that of inulin at an equivalent dosage.

In one embodiment, consumption of food products containing the disclosed soluble α-glucan fiber composition results in a rate of gas production that is well tolerated for food applications. In one embodiment, the relative rate of gas production is no more than the rate observed for inulin under similar conditions, preferably the same or less than inulin, more preferably less than inulin, and most preferably much less than inulin at an equivalent dosage. In another embodiment, the relative rate of gas formation is measured over 3 hours or 24 hours using the methods described herein. In a preferred aspect, the rate of gas formation is at least 1%, preferably 2%, 3%, 4%, 5%, 6%, 7%, 8%, 9%, 10%, 15%, 20%, 25% or at least 30% less than the rate observed for inulin under the same reaction conditions.

### Beneficial Physiological Properties

### Short Chain Fatty Acid Production

Use of the compounds according to the present invention may facilitate the production of energy yielding metabolites through colonic fermentation. Use of compounds according to the invention may facilitate the production of short chain fatty acids (SCFAs), such as propionate and/or butyrate. SCFAs are known to lower cholesterol. Consequently, the compounds of the invention may lower the risk of developing high cholesterol. The disclosed soluble α-glucan fiber composition may stimulate the production of SCFAs, especially proprionate and/or butyrate, in fermentation studies. As the production of SCFAs or the increased ratio of SCFA to acetate is beneficial for the control of cholesterol levels in a mammal in need thereof, the disclosed soluble α-glucan fiber composition may be of particular interest to nutritionists and consumers for the prevention and/or treatment of cardiovascular risks. Thus, in another aspect, the disclosure provides a method for improving the health of a subject comprising administering a composition comprising the disclosed soluble α-glucan fiber composition to a subject in an amount effective to exert a beneficial effect on the health of said subject, such as for treating cholesterol-related diseases. In addition, it is generally known that SCFAs lower the pH in the gut and this helps calcium absorption. Thus, compounds according to the present disclosure may also affect mineral absorption. This means that they may also improve bone health, or prevent or treat osteoporosis by lowering the pH due to SCFA increases in the gut. The production of SCFA may increase viscosity in small intestine which reduces the re-absorption of bile acids; increasing the synthesis of bile acids from cholesterol and reduces circulating low density lipoprotein (LDL) cholesterol.

An “effective amount” of a compound or composition as defined herein refers to an amount effective, at dosages and for periods of time necessary, to achieve a desired beneficial physiological effect, such as lowering of blood cholesterol, increasing short chain fatty acid production or preventing or treating a gastrointestinal disorder. For instance, the amount of a composition administered to a subject will vary depending upon factors such as the subject's condition, the subject's body weight, the age of the subject, and whether a composition is the sole source of nutrition. The effective amount may be readily set by a medical practitioner or dietician. In general, a sufficient amount of the composition is administered to provide the subject with up to about 50 g of dietary fiber (insoluble and soluble) per day; for example about 25 g to about 35 g of dietary fiber per day. The amount of the disclosed soluble α-glucan fiber composition that the subject receives is preferably in the range of about 0.1 g to about 50 g per day, more preferably in the rate of 0.5 g to 20 g per day, and most preferably 1 to 10 g per day. A compound or composition as defined herein may be taken in multiple doses, for example 1 to 5 times, spread out over the day or acutely, or may be taken in a single dose. A compound or composition as defined herein may also be fed continuously over a desired period. In certain embodiments, the desired period is at least one week or at least two weeks or at least three weeks or at least one month or at least six months.

In a preferred embodiment, the disclosure provides a method for decreasing blood triglyceride levels in a subject in need thereof by administering a compound or a composition as defined herein to a subject in need thereof. In another preferred embodiment, the invention provides a method for decreasing low density lipoprotein levels in a subject in need thereof by administering a compound or a composition as defined herein to a subject in need thereof. In another preferred embodiment, the disclosure provides a method for increasing high density lipoprotein levels in a subject in need thereof by administering a compound or a composition as defined herein to a subject in need thereof.

### Attenuation of Postprandial Blood Glucose Concentrations/Glycemic Response

The presence of bonds other than α-(1,4) backbone linkages in the disclosed soluble α-glucan fiber composition provides improved digestion resistance as enzymes of the human digestion track may have difficultly hydrolyzing such bonds and/or branched linkages. The presence of branches provides partial or complete indigestibility to glucan fibers, and therefore virtually no or a slower absorption of glucose into the body, which results in a lower glycemic response. Accordingly, the disclosure provides a soluble α-glucan fiber composition for the manufacture of food and drink compositions resulting in a lower glycemic response. For example, these compounds can be used to replace sugar or other rapidly digestible carbohydrates, and thereby lower the glycemic load of foods, reduce calories, and/or lower the energy density of foods. Also, the stability of the soluble α-glucan fiber composition possessing these types of bonds allows them to be easily passed through into the large intestine where they may serve as a substrate specific for the colonic microbial flora.

### Improvement of Gut Health

In a further embodiment, compounds as disclosed herein may be used for the treatment and/or improvement of gut health. The soluble α-glucan fiber composition is preferably slowly fermented in the gut by the gut microflora. Preferably, the present compounds exhibit in an in vitro gut model a tolerance no worse than inulin or other commercially available fibers such as PROMITOR® (soluble corn fiber, Tate & Lyle), NUTRIOSE® (soluble corn fiber or dextrin, Roquette), or FIBERSOL®-2 (digestion-resistant maltodextrin, Archer Daniels Midland Company & Matsutani Chemical), (i.e., similar level of gas production), preferably an improved tolerance over one or more of the commercially available fibers, i.e. the fermentation of the present glucan fiber results in less gas production than inulin in 3 hours or 24 hours, thereby lowering discomfort, such as flatulence and bloating, due to gas formation. In one aspect, the disclosure also relates to a method for moderating gas formation in the gastrointestinal tract of a subject by administering a compound or a composition as disclosed herein to a subject in need thereof, so as to decrease gut pain or gut discomfort due to flatulence and bloating. In further embodiments, compositions as disclosed herein provide subjects with improved tolerance to food fermentation, and may be combined with fibers, such as inulin or FOS, GOS, or lactulose to improve tolerance by lowering gas production.

In another embodiment, compounds as disclosed herein may be administered to improve laxation or improve regularity by increasing stool bulk.

### Prebiotics and Probiotics

The soluble α-glucan fiber composition(s) may be useful as prebiotics, or as “synbiotics” when used in combination with probiotics, as discussed below. By “prebiotic” it is meant a food ingredient that beneficially affects the subject by selectively stimulating the growth and/or activity of one or a limited number of bacteria in the gastrointestinal tract, particularly the colon, and thus improves the health of the host. Examples of prebiotics include fructooligosaccharides, inulin, polydextrose, resistant starch, soluble corn fiber, glucooligosaccharides and galactooligosaccharides, arabinoxylan-oligosaccharides, lactitol, and lactulose.

In another embodiment, compositions comprising the soluble α-glucan fiber composition further comprise at least one probiotic organism. By “probiotic organism” it is meant living microbiological dietary supplements that provide beneficial effects to the subject through their function in the digestive tract. In order to be effective the probiotic micro-organisms must be able to survive the digestive conditions, and they must be able to colonize the gastrointestinal tract at least temporarily without any harm to the subject. Only certain strains of microorganisms have these properties. Preferably, the probiotic microorganism is selected from the group comprising Lactobacillus spp., Bifidobacterium spp., Bacillus spp., Enterococcus spp., Escherichia spp., Streptococcus spp., and Saccharomyces spp. Specific microorganisms include, but are not limited to Bacillus subtilis, Bacillus cereus, Bifidobacterium bificum, Bifidobacterium breve, Bifidobacterium infantis, Bifidobacterium lactis, Bifidobacterium longum, Bifidobacterium thermophilum, Enterococcus faecium, Enterococcus faecium, Lactobacillus acidophilus, Lactobacillus bulgaricus, Lactobacillus casei, Lactobacillus lactis, Lactobacillus plantarum, Lactobacillus reuteri, Lactobacillus rhamnosus, Streptococcus faecium, Streptococcus mutans, Streptococcus thermophilus, Saccharomyces boulardii, Torulopsia, Aspergillus oryzae, and Streptomyces among others, including their vegetative spores, non-vegetative spores (Bacillus) and synthetic derivatives. More preferred probiotic microorganisms include, but are not limited to members of three bacterial genera: Lactobacillus, Bifidobacterium and Saccharomyces. In a preferred embodiment, the probiotic microorganism is Lactobacillus, Bifidobacterium, and a combination thereof

The probiotic organism can be incorporated into the composition as a culture in water or another liquid or semisolid medium in which the probiotic remains viable. In another technique, a freeze-dried powder containing the probiotic organism may be incorporated into a particulate material or liquid or semi-solid material by mixing or blending.

In a preferred embodiment, the composition comprises a probiotic organism in an amount sufficient to delivery at least 1 to 200 billion viable probiotic organisms, preferably 1 to 100 billion, and most preferably 1 to 50 billion viable probiotic organisms. The amount of probiotic organisms delivery as describe above is may be per dosage and/or per day, where multiple dosages per day may be suitable for some applications. Two or more probiotic organisms may be used in a composition.

### Methods to Obtain the Enzymatically-Produced Soluble α-Glucan Fiber Composition

Any number of common purification techniques may be used to obtain the soluble α-glucan fiber composition from the reaction system including, but not limited to centrifugation, filtration, fractionation, chromatographic separation, dialysis, evaporation, precipitation, dilution or any combination thereof, preferably by dialysis or chromatographic separation, most preferably by dialysis (ultrafiltration).

### Recombinant Microbial Expression

The genes and gene products of the instant sequences may be produced in heterologous host cells, particularly in the cells of microbial hosts. Preferred heterologous host cells for expression of the instant genes and nucleic acid molecules are microbial hosts that can be found within the fungal or bacterial families and which grow over a wide range of temperature, pH values, and solvent tolerances. For example, it is contemplated that any of bacteria, yeast, and filamentous fungi may suitably host the expression of the present nucleic acid molecules. The enzyme(s) may be expressed intracellularly, extracellularly, or a combination of both intracellularly and extracellularly, where extracellular expression renders recovery of the desired protein from a fermentation product more facile than methods for recovery of protein produced by intracellular expression. Transcription, translation and the protein biosynthetic apparatus remain invariant relative to the cellular feedstock used to generate cellular biomass; functional genes will be expressed regardless. Examples of host strains include, but are not limited to, bacterial, fungal or yeast species such as Aspergillus, Trichoderma, Saccharomyces, Pichia, Phaffia, Kluyveromyces, Candida, Hansenula, Yarrowia, Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. In one embodiment, the fungal host cell is Trichoderma, preferably a strain of Trichoderma reesei. In one embodiment, bacterial host strains include Escherichia, Bacillus, Kluyveromyces, and Pseudomonas. In a preferred embodiment, the bacterial host cell is Bacillus subtilis or Escherichia coli.

Large-scale microbial growth and functional gene expression may use a wide range of simple or complex carbohydrates, organic acids and alcohols or saturated hydrocarbons, such as methane or carbon dioxide in the case of photosynthetic or chemoautotrophic hosts, the form and amount of nitrogen, phosphorous, sulfur, oxygen, carbon or any trace micronutrient including small inorganic ions. The regulation of growth rate may be affected by the addition, or not, of specific regulatory molecules to the culture and which are not typically considered nutrient or energy sources.

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell and/or native to the production host, although such control regions need not be so derived.

Initiation control regions or promoters which are useful to drive expression of the present cephalosporin C deacetylase coding region in the desired host cell are numerous and familiar to those skilled in the art. Virtually any promoter capable of driving these genes is suitable for the present invention including, but not limited to, CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred host cell. In one embodiment, the inclusion of a termination control region is optional. In another embodiment, the chimeric gene includes a termination control region derived from the preferred host cell.

### Industrial Production

A variety of culture methodologies may be applied to produce the enzyme(s). For example, large-scale production of a specific gene product over-expressed from a recombinant microbial host may be produced by batch, fed-batch, and continuous culture methodologies. Batch and fed-batch culturing methods are common and well known in the art and examples may be found in Biotechnology: A Textbook of Industrial Microbiology by Wulf Crueger and Anneliese Crueger (authors), Second Edition, (Sinauer Associates, Inc., Sunderland, Mass. (1990) and Manual of Industrial Microbiology and Biotechnology, Third Edition, Richard H. Baltz, Arnold L. Demain, and Julian E. Davis (Editors), (ASM Press, Washington, D.C. (2010).

Commercial production of the desired enzyme(s) may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively, continuous culture may be practiced with immobilized cells where carbon and nutrients are continuously added and valuable products, by-products or waste products are continuously removed from the cell mass. Cell immobilization may be performed using a wide range of solid supports composed of natural and/or synthetic materials.

Recovery of the desired enzyme(s) from a batch fermentation, fed-batch fermentation, or continuous culture, may be accomplished by any of the methods that are known to those skilled in the art. For example, when the enzyme catalyst is produced intracellularly, the cell paste is separated from the culture medium by centrifugation or membrane filtration, optionally washed with water or an aqueous buffer at a desired pH, then a suspension of the cell paste in an aqueous buffer at a desired pH is homogenized to produce a cell extract containing the desired enzyme catalyst. The cell extract may optionally be filtered through an appropriate filter aid such as celite or silica to remove cell debris prior to a heat-treatment step to precipitate undesired protein from the enzyme catalyst solution. The solution containing the desired enzyme catalyst may then be separated from the precipitated cell debris and protein by membrane filtration or centrifugation, and the resulting partially-purified enzyme catalyst solution concentrated by additional membrane filtration, then optionally mixed with an appropriate carrier (for example, maltodextrin, phosphate buffer, citrate buffer, or mixtures thereof) and spray-dried to produce a solid powder comprising the desired enzyme catalyst. Alternatively, the resulting partially-purified enzyme catalyst solution can be stabilized as a liquid formulation by the addition of polyols such as maltodextrin, sorbitol, or propylene glycol, to which is optionally added a preservative such as sorbic acid, sodium sorbate or sodium benzoate.

The production of the soluble α-glucan fiber can be carried out by combining the obtained enzyme(s) under any suitable aqueous reaction conditions which result in the production of the soluble α-glucan fiber such as the conditions disclosed herein. The reaction may be carried out in water solution, or, in certain embodiments, the reaction can be carried out in situ within a food product. Methods for producing a fiber using an enzyme catalyst in situ in a food product are known in the art. In certain embodiments, the enzyme catalyst is added to a sucrose-containing liquid food product. The enzyme catalyst can reduce the amount of sucrose in the liquid food product while increasing the amount of soluble α-glucan fiber and fructose. A suitable method for in situ production of fiber using a polypeptide material (i.e., an enzyme catalyst) within a food product can be found in WO2013/182686, the contents of which are herein incorporated by reference for the disclosure of a method for in situ production of fiber in a food product using an enzyme catalyst.

When an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

### DESCRIPTION OF CERTAIN EMBODIMENTS

In a first embodiment (the “first embodiment”), a soluble α-glucan fiber composition is provided, said soluble α-glucan fiber composition comprising:
a. at least 75% α-(1,3) glycosidic linkages, preferably at least 80%, more preferably at least 85%, even more preferably at least 90%, and most preferably at least 95% α-(1,3) glycosidic linkages;
b. less than 25% α-(1,6) glycosidic linkages; preferably less than 10%, more preferably 5% or less, and even more preferably less than 1% α-(1,6) glycosidic linkages;
c. less than 10% α-(1,3,6) glycosidic linkages; preferably less than 5%, and most preferably less than 2.5% α-(1,3,6) glycosidic linkages;
d. a weight average molecular weight of less than 5000 Daltons; preferably less than 2500 Daltons, more preferably between 500 and 2500 Daltons, and most preferably about 500 to about 2000 Daltons;
e. a viscosity of less than 0.25 Pascal second (Pa·s), preferably less than 0.01 Pascal second (Pa·s), preferably less than 7 cP (0.007 Pa·s), more preferably less than 5 cP (0.005 Pa·s), more preferably less than 4 cP (0.004 Pa·s), and most preferably less than 3 cP (0.003 Pa·s) at 12 wt % in water at 20° C.
f. a dextrose equivalence (DE) in the range of 4 to 40, preferably 10 to 40, and
g. a digestibility of less than 12%, preferably less than 11%, 10%, 9%, 8%, 7%, 6%, 5%, 4%, 3%, 2% or 1% digestible as measured by the Association of Analytical Communities (AOAC) method 2009.01;
h. a solubility of at least 20% (w/w), preferably at least 30%, 40%, 50%, 60%, or 70%, in water at 25° C.; and
i. a polydispersity index of less than 5.

In a second embodiment, a carbohydrate composition is provided comprising 0.01 to 99 wt % (dry solids basis), preferably 10 to 90% wt %, of the soluble α-glucan fiber composition described above.

In a third embodiment, a food product, personal care product or pharmaceutical product is provided comprising the soluble α-glucan fiber composition of the first embodiment or a carbohydrate composition comprising the soluble α-glucan fiber composition of the second embodiment.

In another embodiment, a low cariogenicity composition is provided comprising the soluble α-glucan fiber composition of the first embodiment and at least one polyol.

In another embodiment, a method is provided to produce a soluble α-glucan fiber composition comprising:
a. providing a set of reaction components comprising:
   a) sucrose;
   b) at least one glucosyltransferase capable of catalyzing the synthesis of glucan polymers having at least 75%, preferably at least 80%, more preferably at least 85%, even more preferably at least 90%, and most preferably at least 95% α-(1,3) glycosidic linkages;
   c) at least one α-glucanohydrolase capable of hydrolyzing glucan polymers having one or more α-(1,3) glycosidic linkages or one or more α-(1,6) glycosidic linkages; and
   d) optionally one or more acceptors;
b. combining the set of reaction components under suitable aqueous reaction conditions whereby a product comprising a soluble α-glucan fiber composition is produced; and
c. optionally isolating the soluble α-glucan fiber composition from the product of step (b); and
d. optionally concentrating the isolated soluble α-glucan fiber composition of step (c).

In another embodiment, the soluble α-glucan fiber composition produced by the above method comprises:

a. a viscosity of less than 0.01 Pascal second (Pa·s) at 12 wt % in water 20° C.;

b. a digestibility of less than 10% as measured by the Association of Analytical Communities (AOAC) method 2009.01;

c. a solubility of at least 20% (w/w) in water at 25° C.; and

d. a polydispersity index of less than 5.

In another embodiment, a method is provided to produce the soluble α-glucan fiber composition of the first embodiment comprising:
a. providing a set of reaction components comprising:
   a) sucrose;
   b) at least one glucosyltransferase capable of catalyzing the synthesis of glucan polymers having at least 75%, preferably at least 80%, more preferably at least 85%, even more preferably at least 90%, and most preferably at least 95% α-(1,3) glycosidic linkages;
   c) at least one α-glucanohydrolase capable of hydrolyzing glucan polymers having one or more α-(1,3) glycosidic linkages or one or more α-(1,6) glycosidic linkages; and
   d) optionally one more acceptors;
b. combining the set of reaction components under suitable aqueous reaction conditions to form a single reaction mixture, whereby a product mixture comprising glucose oligomers is formed;
c. isolating the soluble α-glucan fiber composition of the first embodiment from the product mixture comprising glucose oligomers; and
d. optionally concentrating the soluble α-glucan fiber composition.

In another embodiment, a method is provided to make a blended carbohydrate composition comprising combining the soluble α-glucan fiber composition of the first embodiment with one or more of the following: a monosaccharide, a disaccharide, glucose, sucrose, fructose, leucrose, corn syrup, high fructose corn syrup, isomerized sugar, maltose, trehalose, panose, raffinose, cellobiose, isomaltose, honey, maple sugar, a fruit-derived sweetener, sorbitol, maltitol, isomaltitol, lactose, nigerose, kojibiose, xylitol, erythritol, dihydrochalcone, stevioside, α-glycosyl stevioside, acesulfame potassium, alitame, neotame, glycyrrhizin, thaumantin, sucralose, L-aspartyl-L-phenylalanine methyl ester, saccharine, maltodextrin, starch, potato starch, tapioca starch, dextran, soluble corn fiber, a resistant maltodextrin, a branched maltodextrin, inulin, polydextrose, a fructooligosaccharide, a galactooligosaccharide, a xylooligosaccharide, an arabinoxylooligosaccharide, a nigerooligosaccharide, a gentiooligosaccharide, hemicellulose, fructose oligomer syrup, an isomaltooligosaccharide, a filler, an excipient, a binder, or any combination thereof.

In another embodiment, a method to make a food product, personal care product, or pharmaceutical product is provided comprising mixing one or more edible food ingredients, cosmetically acceptable ingredients or pharmaceutically acceptable ingredients; respectively, with the soluble α-glucan fiber composition of the first embodiment, the carbohydrate composition of the second embodiment, or a combination thereof.

In another embodiment, a method to reduce the glycemic index of a food or beverage is provided comprising incorporating into the food or beverage the soluble α-glucan fiber composition of the first embodiment.

In another embodiment, a method of inhibiting the elevation of blood-sugar level, lowering lipids in the living body, treating constipation or reducing gastrointestinal transit time is provided comprising a step of administering the soluble α-glucan fiber composition of the first embodiment to a mammal.

In another embodiment, a use of the soluble α-glucan fiber composition of the first embodiment in a food composition suitable for consumption by humans and animals is also provided.

Also provided are compositions or methods according to any of the above embodiments wherein the soluble α-glucan fiber composition comprises a reducing sugars content of less than 10%, preferably less than 5 wt %, and most preferably 1 wt % or less.

Also provided are compositions or methods according to any of the above embodiments wherein the soluble α-glucan fiber composition comprises less than 5%, or less than 3%, preferably less than 1%, and most preferably less than 0.5% α-(1,4) glycosidic linkages.

Also provided are compositions or methods according to any of the above embodiments wherein the carbohydrate composition comprising at least one of the following: a monosaccharide, a disaccharide, glucose, sucrose, fructose, leucrose, corn syrup, high fructose corn syrup, isomerized sugar, maltose, trehalose, panose, raffinose, cellobiose, isomaltose, honey, maple sugar, a fruit-derived sweetener, sorbitol, maltitol, isomaltitol, lactose, nigerose, kojibiose, xylitol, erythritol, dihydrochalcone, stevioside, α-glycosyl stevioside, acesulfame potassium, alitame, neotame, glycyrrhizin, thaumantin, sucralose, L-aspartyl-L-phenylalanine methyl ester, saccharine, maltodextrin, starch, potato starch, tapioca starch, dextran, soluble corn fiber, a resistant maltodextrin, a branched maltodextrin, inulin, polydextrose, a fructooligosaccharide, a galactooligosaccharide, a xylooligosaccharide, an arabinoxylooligosaccharide, a nigerooligosaccharide, a gentiooligosaccharide, hemicellulose, fructose oligomer syrup, an isomaltooligosaccharide, a filler, an excipient, a binder, or any combination thereof.

Also provided are compositions or methods according to any of the above embodiments wherein the carbohydrate composition is in the form of a liquid, a syrup, a powder, granules, shaped spheres, shaped sticks, shaped plates, shaped cubes, tablets, powders, capsules, sachets, or any combination thereof.

Also provided are compositions or methods according to any of the above embodiments where the food product is
a. a bakery product selected from the group consisting of cakes, brownies, cookies, cookie crisps, muffins, breads, and sweet doughs, extruded cereal pieces, and coated cereal pieces;
b. a dairy product selected from the group consisting of yogurt, yogurt drinks, milk drinks, flavored milks, smoothies, ice cream, shakes, cottage cheese, cottage cheese dressing, quarg, and whipped mousse-type products.;
c. confections selected from the group consisting of hard candies, fondants, nougats and marshmallows, gelatin jelly candies, gummies, jellies, chocolate, licorice, chewing gum, caramels, toffees, chews, mints, tableted confections, and fruit snacks;
d. beverages selected from the group consisting of carbonated beverages, fruit juices, concentrated juice mixes, clear waters, and beverage dry mixes;
e. high solids fillings for snack bars, toaster pastries, donuts, or cookies;
f. extruded and sheeted snacks selected from the group consisting of puffed snacks, crackers, tortilla chips, and corn chips;
g. snack bars, nutrition bars, granola bars, protein bars, and cereal bars;
h. cheeses, cheese sauces, and other edible cheese products;
i. edible films;
j. water soluble soups, syrups, sauces, dressings, or coffee creamers; or
k. dietary supplements; preferably in the form of tablets, powders, capsules or sachets.

Also provided are compositions or methods according to any of the embodiments wherein the α-glucanohydrolase is an endomutanase and the glucosyltransferase is a mutansucrase.

Also provided are compositions comprising 0.01 to 99 wt % (dry solids basis) of the disclosed soluble α-glucan fiber composition and at least one of the following ingredients: a synbiotic, a peptide, a peptide hydrolysate, a protein, a protein hydrolysate, a soy protein, a dairy protein, an amino acid, a polyol, a polyphenol, a vitamin, a mineral, an herbal, an herbal extract, a fatty acid, a polyunsaturated fatty acid (PUFAs), a phytosteroid, betaine, carotenoid, a digestive enzyme, a probiotic organism or any combination thereof.

Also provided are methods according to any of the embodiments wherein the isolating step comprises at least one of centrifugation, filtration, fractionation, chromatographic separation, dialysis, evaporation, dilution or any combination thereof.

Also provided are methods according to any of the embodiments wherein the sucrose concentration in the single reaction mixture is initially at least 200 g/L upon combining the set of reaction components.

Also provided are methods according to any of the embodiments wherein the ratio of glucosyltransferase to α-glucanohydrolase (v/v) ranges from 0.01:1 to 1:0.01. In other embodiments, the ratio of glucosyltransferase to α-glucanhydrolase (units/units) ranges from 0.01:1 to 1:0.01.

Also provided are methods according to any of the embodiments wherein the suitable reaction conditions comprise a reaction temperature between 0° C. and 55° C.

Also provided are methods according to any of the embodiments wherein the suitable reaction conditions comprise a pH range of 4 to 8.

Also provided are methods according to any of the above embodiments, wherein combining the set of reaction components under suitable aqueous reaction conditions comprises combining the reaction components in water.

Also provided are methods according to any of the above embodiments, wherein combining the set of reaction components under suitable aqueous reaction conditions comprises combining the reaction components within a food product.

Also provided are methods according to any of the above embodiments wherein the suitable reactions conditions comprise including a buffer that is selected from the group consisting of phosphate, pyrophosphate, bicarbonate, acetate, and citrate.

Also provided are methods according to any of the above embodiments wherein said at least one glucosyltransferase comprises an amino acid sequence is SEQ ID NOs: 3, 5, 17, 19, 88, 90, 92, 94, 96, 98, 100, 102, 104, 106, 108, 110, 112, or a combination thereof. In other embodiments, the at least one glucosyl transferase is GTF-S, a truncation thereof, a homolog thereof, or a trucation of a homolog thereof. In another embodiment, the glucosyltransferase is a truncation of GTF-S and comprises the amino acid sequence of SEQ ID NO: 126. In other embodiments, the glucosyl transferase is a truncation of a homolog of GTF-S and comprises an amino acid sequence is SEQ ID NO: 118, 120, 122, 124, 128, 130, 132, 134, 136, 138, 140, 142, 144, 146, 146, 148, 150, 152 or a combination thereof

Also provided are methods according to any of the above embodiments wherein said at least one α-glucanohydrolase comprises an amino acid sequence is SEQ ID NOs 21, 22, 24, 27, 54, 56, 58, or a combination thereof.

Also provided is a method according to any of the above embodiments wherein said at least one glucosyltransferase and said at least one α-glucanohydrolase comprise amino acid sequences having at least 90% identity to sequences selected from the following combinations of sequences and truncations thereof:
1) glucosyltransferase GTF7527 (SEQ ID NOs: 3, 5 or a combination thereof) and mutanase MUT3325 (SEQ ID NO: 27)
2) glucosyltransferase GTF7527 (SEQ ID NOs: 3, 5 or a combination thereof) and mutanase MUT3264 (SEQ IDs NO: 21, 22, 24 or any combination thereof);
3) glucosyltransferase GTF0459 (SEQ ID NOs: 17, 19 or a combination thereof) or homologs of GTF0459 (SEQ ID NOs: 88, 90, 92, 94, 96, 98, 100, 102, 104, 106, 108, 110, and 112) and mutanase MUT3325 (SEQ ID NO: 27); and
4) glucosyltransferase GTF0459 (SEQ ID NO: 17, 19 or a combination thereof) or homologs of GTF0459 (SEQ ID NOs: 88, 90, 92, 94, 96, 98, 100, 102, 104, 106, 108, 110, and 112) and mutanase MUT3264 (SEQ ID NO: 21, 22, 24 or any combination thereof).

In another embodiment, a method to produce the soluble α-glucan fiber composition of the first embodiment is provided comprising:
a. providing a set of reaction components comprising:
   i. sucrose;
   ii. at least one glucosyltransferase capable of catalyzing the synthesis of glucan polymers having one or more α-(1,3) glycosidic linkages;
   iii. optionally one more acceptors;
b. combining under suitable aqueous reaction conditions the set of reaction components of (a) to form a single reaction mixture, wherein the reaction conditions comprise a reaction temperature greater than 45° C. and less than 55° C., preferably 47° C. to 53° C., whereby a product mixture comprising glucose oligomers is formed;
c. isolating the soluble α-glucan fiber composition of claim 1 from the product mixture comprising glucose oligomers; and
d. optionally concentrating the soluble α-glucan fiber composition.

In another embodiment, a method according to any of the above embodiments is provided wherein the glucosyltransferase is obtained from Streptococcus salivarius, preferably having an amino acid sequence selected from SEQ ID NOs: 3, 5 and a combination thereof.

In another embodiment, a product produced by any of the above process embodiments is provided; preferably wherein the product produced is the soluble α-glucan fiber composition of the first embodiment.

### EXAMPLES

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention.

The present invention is further defined in the following Examples. It should be understood that these Examples, while indicating preferred embodiments of the invention, are given by way of illustration only and should not be considered to limit the scope of the claims. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “sec” or “s” means second(s), “ms” mean milliseconds, “min” means minute(s), “h” or “hr” means hour(s), “μL” means microliter(s), “mL” means milliliter(s), “L” means liter(s); “mL/min” is milliliters per minute; “μg/mL” is microgram(s) per milliliter(s); “LB” is Luria broth; “μm” is micrometers, “nm” is nanometers; “OD” is optical density; “IPTG” is isopropyl-β-D-thio-galactoside; “g” is gravitational force; “mM” is millimolar; “SDS-PAGE” is sodium dodecyl sulfate polyacrylamide; “mg/mL” is milligrams per milliliters; “N” is normal; “w/v” is weight for volume; “DTT” is dithiothreitol; “BCA” is bicinchoninic acid; “DMAc” is N,N′-dimethyl acetamide; “LiCl” is Lithium chloride’ “NMR” is nuclear magnetic resonance; “DMSO” is dim ethylsulfoxide; “SEC” is size exclusion chromatography; “GI” or “gi” means GenInfo Identifier, a system used by GENBANK® and other sequence databases to uniquely identify polynucleotide and/or polypeptide sequences within the respective databases; “DPx” means glucan degree of polymerization having “x” units in length; “ATCC” means American Type Culture Collection (Manassas, Va.), “DSMZ” and “DSM” will refer to Leibniz Institute DSMZ-German Collection of Microorganisms and Cell Cultures, (Braunschweig, Germany); “EELA” is the Finish Food Safety Authority (Helsinki, Finland;) “CCUG” refer to the Culture Collection, University of Göteborg, Sweden; “Suc.” means sucrose; “Gluc.” means glucose; “Fruc.” means fructose; “Leuc.” means leucrose; and “Rxn” means reaction.

### General Methods

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., (American Society for Microbiology Press, Washington, D.C. (1994)), Biotechnology: A Textbook of Industrial Microbiology by Wulf Crueger and Anneliese Crueger (authors), Second Edition, (Sinauer Associates, Inc., Sunderland, Mass. (1990)), and Manual of Industrial Microbiology and Biotechnology, Third Edition, Richard H. Baltz, Arnold L. Demain, and Julian E. Davis (Editors), (American Society of Microbiology Press, Washington, D.C. (2010).

All reagents, restriction enzymes and materials used for the growth and maintenance of bacterial cells were obtained from BD Diagnostic Systems (Sparks, Md.), Invitrogen/Life Technologies Corp. (Carlsbad, Calif.), Life Technologies (Rockville, Md.), QIAGEN (Valencia, Calif.), Sigma-Aldrich Chemical Company (St. Louis, Mo.) or Pierce Chemical Co. (A division of Thermo Fisher Scientific Inc., Rockford, Ill.) unless otherwise specified. IPTG, (cat #I6758) and triphenyltetrazolium chloride were obtained from the Sigma Co., (St. Louis, Mo.). Bellco spin flask was from the Bellco Co., (Vineland, N.J.). LB medium was from Becton, Dickinson and Company (Franklin Lakes, N.J.). BCA protein assay was from Sigma-Aldrich (St Louis, Mo.).

### Growth of Recombinant E. Coli Strains for Production of GTF Enzymes

Escherichia coli strains expressing a functional GTF enzyme were grown in shake flask using LB medium with ampicillin (100 μg/mL) at 37° C. and 220 rpm to OD_{600 nm}=0.4-0.5, at which time isopropyl-β-D-thio-galactoside (IPTG) was added to a final concentration of 0.5 mM and incubation continued for 2-4 hr at 37° C. Cells were harvested by centrifugation at 5,000×g for 15 min and resuspended (20%-25% wet cell weight/v) in 50 mM phosphate buffer pH 7.0). Resuspended cells were passed through a French Pressure Cell (SLM Instruments, Rochester, N.Y.) twice to ensure >95% cell lysis. Cell lysate was centrifuged for 30 min at 12,000×g and 4° C. The resulting supernatant (cell extract) was analyzed by the BCA protein assay and SDS-PAGE to confirm expression of the GTF enzyme, and the cell extract was stored at −80° C.

pHYT Vector

The pHYT vector backbone is a replicative Bacillus subtilis expression plasmid containing the Bacillus subtilis aprE promoter. It was derived from the Escherichia coli-Bacillus subtilis shuttle vector pHY320PLK (GENBANK® Accession No. D00946 and is commercially available from Takara Bio Inc. (Otsu, Japan)). The replication origin for Escherichia coli and ampicillin resistance gene are from pACYC177 (GENBANK® X06402 and is commercially available from New England Biolabs Inc., Ipswich, Mass.). The replication origin for Bacillus subtilis and tetracycline resistance gene were from pAMalpha-1 (Francia et al., J Bacteriol. 2002 September; 184(18):5187-93)). To construct pHYT, a terminator sequence: 5′-ATAAAAAACGCTCGGTTGCCGCCGGGCGTTTTTTAT-3′ (SEQ ID NO: 1) from phage lambda was inserted after the tetracycline resistance gene. The entire expression cassette (EcoRI-BamHI fragment) containing the aprE promoter-AprE signal peptide sequence-coding sequence encoding the enzyme of interest (e.g., coding sequences for various GTFs)-BPN′ terminator was cloned into the EcoRI and HindIII sites of pHYT using a BamHI-HindIII linker that destroyed the HindIII site. The linker sequence is 5′-GGATCCTGACTGCCTGAGCTT-3′ (SEQ ID NO: 2). The aprE promoter and AprE signal peptide sequence (SEQ ID NO: 25) are native to Bacillus subtilis. The BPN′ terminator is from subtilisin of Bacillus amyloliquefaciens. In the case when native signal peptide was used, the AprE signal peptide was replaced with the native signal peptide of the expressed gene.

### Biolistic Transformation of T. Reesei

A Trichoderma reesei spore suspension was spread onto the center ˜6 cm diameter of an acetamidase transformation plate (150 μL of a 5×10⁷-5×10⁸ spore/mL suspension). The plate was then air dried in a biological hood. The stopping screens (BioRad 165-2336) and the macrocarrier holders (BioRad 1652322) were soaked in 70% ethanol and air dried. DRIERITE® desiccant (calcium sulfate desiccant; W. A. Hammond DRIERITE® Company, Xenia, Ohio) was placed in small Petri dishes (6 cm Pyrex) and overlaid with Whatman filter paper (GE Healthcare Bio-Sciences, Pittsburgh, Pa.). The macrocarrier holder containing the macrocarrier (BioRad 165-2335; Bio-Rad Laboratories, Hercules, Calif.) was placed flatly on top of the filter paper and the Petri dish lid replaced. A tungsten particle suspension was prepared by adding 60 mg tungsten M-10 particles (microcarrier, 0.7 micron, BioRad #1652266, Bio-Rad Laboratories) to an Eppendorf tube. Ethanol (1 mL) (100%) was added. The tungsten was vortexed in the ethanol solution and allowed to soak for 15 minutes. The Eppendorf tube was microfuged briefly at maximum speed to pellet the tungsten. The ethanol was decanted and washed three times with sterile distilled water. After the water wash was decanted the third time, the tungsten was resuspended in 1 mL of sterile 50% glycerol. The transformation reaction was prepared by adding 25 μL suspended tungsten to a 1.5 mL-Eppendorf tube for each transformation. Subsequent additions were made in order, 2 μL DNA pTrex3 expression vectors (see U.S. Pat. No. 6,426,410), 25 μL 2.5M CaCl2, 10 μL 0.1M spermidine. The reaction was vortexed continuously for 5-10 minutes, keeping the tungsten suspended. The Eppendorf tube was then microfuged briefly and decanted. The tungsten pellet was washed with 200 μL of 70% ethanol, microfuged briefly to pellet and decanted. The pellet was washed with 200 μL of 100% ethanol, microfuged briefly to pellet, and decanted. The tungsten pellet was resuspended in 24 μL 100% ethanol. The Eppendorf tube was placed in an ultrasonic water bath for 15 seconds and 8 μL aliquots were transferred onto the center of the desiccated macrocarriers. The macrocarriers were left to dry in the desiccated Petri dishes.

A Helium tank was turned on to 1500 psi (˜10.3 MPa). 1100 psi (˜7.58 MPa) rupture discs (BioRad 165-2329) were used in the Model PDS-1000/He™ BIOLISTIC® Particle Delivery System (BioRad). When the tungsten solution was dry, a stopping screen and the macrocarrier holder were inserted into the PDS-1000. An acetamidase plate, containing the target T. reesei spores, was placed 6 cm below the stopping screen. A vacuum of 29 inches Hg (˜98.2 kPa) was pulled on the chamber and held. The He BIOLISTIC® Particle Delivery System was fired. The chamber was vented and the acetamidase plate removed for incubation at 28° C. until colonies appeared (5 days).

Modified amdS Biolistic Agar (MABA) per Liter
Part I, make in 500 mL distilled water (dH₂O)
1000× salts 1 mL
Noble agar 20 g
pH to 6.0, autoclave
Part II, make in 500 mL dH₂O
Acetamide 0.6 g
CsCl 1.68 g
Glucose 20 g
KH₂PO₄ 15 g
MgSO₄.7H₂O 0.6 g
CaCl₂.2H₂O 0.6 g
pH to 4.5, 0.2 micron filter sterilize; leave in 50° C. oven to warm, add to agar, mix, pour plates. Stored at room temperature (˜21° C.)

### 1000× Salts per Liter

FeSO₄.7H₂O 5 g
MnSO₄.H₂O 1.6 g
ZnSO₄.7H₂O 1.4 g
CoCl₂.6H₂O 1 g
Bring up to 1 L dH₂O.
0.2 micron filter sterilize

### Determination of the Glucosyltransferase Activity

Glucosyltransferase activity assay was performed by incubating 1-10% (v/v) crude protein extract containing GTF enzyme with 200 g/L sucrose in 25 mM or 50 mM sodium acetate buffer at pH 5.5 in the presence or absence of 25 g/L dextran (MW˜1500, Sigma-Aldrich, Cat. #31394) at 37° C. and 125 rpm orbital shaking. One aliquot of reaction mixture was withdrawn at 1 h, 2 h and 3 h and heated at 90° C. for 5 min to inactivate the GTF. The insoluble material was removed by centrifugation at 13,000×g for 5 min, followed by filtration through 0.2 μm RC (regenerated cellulose) membrane. The resulting filtrate was analyzed by HPLC using two Aminex HPX-87C columns series at 85° C. (Bio-Rad, Hercules, Calif.) to quantify sucrose concentration. The sucrose concentration at each time point was plotted against the reaction time and the initial reaction rate was determined from the slope of the linear plot. One unit of GTF activity was defined as the amount of enzyme needed to consume one micromole of sucrose in one minute under the assay condition.

### Determination of the α-Glucanohydrolase Activity

Insoluble mutan polymers required for determining mutanase activity were prepared using secreted enzymes produced by Streptococcus sobrinus ATCC® 33478™. Specifically, one loop of glycerol stock of S. sobrinus ATCC® 33478™ was streaked on a BHI agar plate (Brain Heart Infusion agar, Teknova, Hollister, Calif.), and the plate was incubated at 37° C. for 2 days; A few colonies were picked using a loop to inoculate 2×100 mL BHI liquid medium in the original medium bottle from Teknova, and the culture was incubated at 37° C., static for 24 h. The resulting cells were removed by centrifugation and the resulting supernatant was filtered through 0.2 μm sterile filter; 2×101 mL of filtrate was collected. To the filtrate was added 2×11.2 mL of 200 g/L sucrose (final sucrose 20 g/L). The reaction was incubated at 37° C., with no agitation for 67 h. The resulting polysaccharide polymers were collected by centrifugation at 5000×g for 10 min. The supernatant was carefully decanted. The insoluble polymers were washed 4 times with 40 mL of sterile water. The resulting mutan polymers were lyophilized for 48 h. Mutan polymer (390 mg) was suspended in 39 mL of sterile water to make suspension of 10 mg/mL. The mutan suspension was homogenized by sonication (40% amplitude until large lumps disappear, ˜10 min in total). The homogenized suspension was aliquoted and stored at 4° C.

A mutanase assay was initiated by incubating an appropriate amount of enzyme with 0.5 mg/mL mutan polymer (prepared as described above) in 25 mM KOAc buffer at pH 5.5 and 37° C. At various time points, an aliquot of reaction mixture was withdrawn and quenched with equal volume of 100 mM glycine buffer (pH 10). The insoluble material in each quenched sample was removed by centrifugation at 14,000×g for 5 min. The reducing ends of oligosaccharide and polysaccharide polymer produced at each time point were quantified by the p-hydroxybenzoic acid hydrazide solution (PAHBAH) assay (Lever M., Anal. Biochem., (1972) 47:273-279) and the initial rate was determined from the slope of the linear plot of the first three or four time points of the time course. The PAHBAH assay was performed by adding 10 μL of reaction sample supernatant to 100 μL of PAHBAH working solution and heated at 95° C. for 5 min. The working solution was prepared by mixing one part of reagent A (0.05 g/mL p-hydroxy benzoic acid hydrazide and 5% by volume of concentrated hydrochloric acid) and four parts of reagent B (0.05 g/mL NaOH, 0.2 g/mL sodium potassium tartrate). The absorption at 410 nm was recorded and the concentration of the reducing ends was calculated by subtracting appropriate background absorption and using a standard curve generated with various concentrations of glucose as standards. A Unit of mutanase activity is defined as the conversion of 1 micromole/min of mutan polymer at pH 5.5 and 37° C., determined by measuring the increase in reducting ends as described above.

### Determination of Glycosidic Linkages

One-dimensional ¹H NMR data were acquired on a Varian Unity Inova system (Agilent Technologies, Santa Clara, Calif.) operating at 500 MHz using a high sensitivity cryoprobe. Water suppression was obtained by carefully placing the observe transmitter frequency on resonance for the residual water signal in a “presat” experiment, and then using the “tnnoesy” experiment with a full phase cycle (multiple of 32) and a mix time of 10 ms.

Typically, dried samples were taken up in 1.0 mL of D₂O and sonicated for 30 min. From the soluble portion of the sample, 100 μL was added to a 5 mm NMR tube along with 350 μL D₂O and 100 μL of D₂O containing 15.3 mM DSS (4,4-dimethyl-4-silapentane-1-sulfonic acid sodium salt) as internal reference and 0.29% NaN₃ as bactericide. The abundance of each type of anomeric linkage was measured by the integrating the peak area at the corresponding chemical shift. The percentage of each type of anomeric linkage was calculated from the abundance of the particular linkage and the total abundance anomeric linkages from oligosaccharides.

### Methylation Analysis

The distribution of glucosidic linkages in glucans was determined by a well-known technique generally named “methylation analysis,” or “partial methylation analysis” (see: F. A. Pettolino, et al., Nature Protocols, (2012) 7(9):1590-1607). The technique has a number of minor variations but always includes: 1. methylation of all free hydroxyl groups of the glucose units, 2. hydrolysis of the methylated glucan to individual monomer units, 3. reductive ring-opening to eliminate anomers and create methylated glucitols; the anomeric carbon is typically tagged with a deuterium atom to create distinctive mass spectra, 4. acetylation of the free hydroxyl groups (created by hydrolysis and ring opening) to create partially methylated glucitol acetates, also known as partially methylated products, 5. analysis of the resulting partially methylated products by gas chromatography coupled to mass spectrometry and/or flame ionization detection.

The partially methylated products include non-reducing terminal glucose units, linked units and branching points. The individual products are identified by retention time and mass spectrometry. The distribution of the partially-methylated products is the percentage (area %) of each product in the total peak area of all partially methylated products. The gas chromatographic conditions were as follows: RTx-225 column (30 m×250 μm ID×0.1 μm film thickness, Restek Corporation, Bellefonte, Pa., USA), helium carrier gas (0.9 mL/min constant flow rate), oven temperature program starting at 80° C. (hold for 2 min) then 30° C./min to 170° C. (hold for 0 min) then 4° C./min to 240° C. (hold for 25 min), 1 μL injection volume (split 5:1), detection using electron impact mass spectrometry (full scan mode)

### Viscosity Measurement

The viscosity of 12 wt % aqueous solutions of soluble fiber was measured using a TA Instruments AR-G2 controlled-stress rotational rheometer (TA Instruments—Waters, LLC, New Castle, Del.) equipped with a cone and plate geometry. The geometry consists of a 40 mm 2° upper cone and a peltier lower plate, both with smooth surfaces. An environmental chamber equipped with a water-saturated sponge was used to minimize solvent (water) evaporation during the test. The viscosity was measured at 20° C. The peltier was set to the desired temperature and 0.65 mL of sample was loaded onto the plate using an Eppendorf pipette (Eppendorf North America, Hauppauge, N.Y.). The cone was lowered to a gap of 50 μm between the bottom of the cone and the plate. The sample was thermally equilibrated for 3 minutes. A shear rate sweep was performed over a shear rate range of 500-10 s⁻¹. Sample stability was confirmed by running repeat shear rate points at the end of the test.

### Determination of the Concentration of Sucrose, Glucose, Fructose and Leucrose

Sucrose, glucose, fructose, and leucrose were quantitated by HPLC with two tandem Aminex HPX-87C Columns (Bio-Rad, Hercules, Calif.). Chromatographic conditions used were 85° C. at column and detector compartments, 40° C. at sample and injector compartment, flow rate of 0.6 mL/min, and injection volume of 10 μL. Software packages used for data reduction were EMPOWER™ version 3 from Waters (Waters Corp., Milford, Mass.). Calibrations were performed with various concentrations of standards for each individual sugar.

### Determination of the Concentration of Oligosaccharides

Soluble oligosaccharides were quantitated by HPLC with two tandem Aminex HPX-42A columns (Bio-Rad). Chromatographic conditions used were 85° C. column temperature and 40° C. detector temperature, water as mobile phase (flow rate of 0.6 mL/min), and injection volume of 10 μL. Software package used for data reduction was EMPOWER™ version 3 from Waters Corp. Oligosaccharide samples from DP2 to DP7 were obtained from Sigma-Aldrich: maltoheptaose (DP7, Cat. #47872), maltohexanose (DP6, Cat. #47873), maltopentose (DP5, Cat. #47876), maltotetraose (DP4, Cat. #47877), isomaltotriose (DP3, Cat. #47884) and maltose (DP2, Cat. #47288). Calibration was performed for each individual oligosaccharide with various concentrations of the standard.

### Determination of Digestibility

The digestibility test protocol was adapted from the Megazyme Integrated Total Dietary Fiber Assay (AOAC method 2009.01, Ireland). The final enzyme concentrations were kept the same as the AOAC method: 50 Unit/mL of pancreatic α-amylase (PAA), 3.4 Units/mL for amyloglucosidase (AMG). The substrate concentration in each reaction was 25 mg/mL as recommended by the AOAC method. The total volume for each reaction was 1 mL instead of 40 mL as suggested by the original protocol. Every sample was analyzed in duplicate with and without the treatment of the two digestive enzymes. The detailed procedure is described below:

The enzyme stock solution was prepared by dissolving 20 mg of purified porcine pancreatic α-amylase (150,000 Units/g; AOAC Method 2002.01) from the Integrated Total Dietary Fiber Assay Kit in 29 mL of sodium maleate buffer (50 mM, pH 6.0 plus 2 mM CaCl₂) and stir for 5 min, followed by the addition of 60 uL amyloglucosidase solution (AMG, 3300 Units/mL) from the same kit. 0.5 mL of the enzyme stock solution was then mixed with 0.5 mL soluble fiber sample (50 mg/mL) in a glass vial and the digestion reaction mixture was incubated at 37° C. and 150 rpm in orbital motion in a shaking incubator for exactly 16 h. Duplicated reactions were performed in parallel for each fiber sample. The control reactions were performed in duplicate by mixing 0.5 mL maleate buffer (50 mM, pH 6.0 plus 2 mM CaCl₂) and 0.5 mL soluble fiber sample (50 mg/mL) and reaction mixtures was incubated at 37° C. and 150 rpm in orbital motion in a shaking incubator for exactly 16 h. After 16 h, all samples were removed from the incubator and immediately 75 μL of 0.75 M TRIZMA® base solution was added to terminate the reaction. The vials were immediately placed in a heating block at 95-100° C., and incubate for 20 min with occasional shaking (by hand). The total volume of each reaction mixture is 1.075 mL after quenching. The amount of released glucose in each reaction was quantified by HPLC with the Aminex HPX-87C Columns (BioRad) as described in the General Methods. Maltodextrin (DE4-7, Sigma Aldrich, St. Louis, Mo.) was used as the positive control for the enzymes. To calculate the digestibility, the following formula was used: Digestibility=100%*[amount of glucose (mg) released after treatment with enzyme−amount of glucose (mg) released in the absence of enzyme]/1.1*amount of total fiber (mg)”

### Purification of Soluble Oligosaccharide Fiber

Soluble oligosaccharide fiber present in product mixtures produced by the conversion of sucrose using glucosyltransferase enzymes with or without added mutanases as described in the following examples were purified and isolated by size-exclusion column chromatography (SEC). In a typical procedure, product mixtures were heat-treated at 60° C. to 90° C. for between 15 min and 30 min and then centrifuged at 4000 rpm for 10 min. The resulting supernatant was injected onto an ÄKTAprime purification system (SEC; GE Healthcare Life Sciences) (10 mL-50 mL injection volume) connected to a GE HK 50/60 column packed with 1.1 L of Bio-Gel P2 Gel (Bio-Rad, Fine 45-90 μm) using water as eluent at 0.7 mL/min. The SEC fractions (˜5 mL per tube) were analyzed by HPLC for oligosaccharides using a Bio-Rad HPX-47A column. Fractions containing >DP2 oligosaccharides were combined and the soluble fiber isolated by rotary evaporation of the combined fractions to produce a solution containing between 3% and 6% (w/w) solids, where the resulting solution was lyophilized to produce the soluble fiber as a solid product.

### Pure Culture Growth on Specific Carbon Sources

To test the capability of microorganisms to grow on specific carbon sources (oligosaccharide or polysaccharide soluble fibers), selected microbes were grown in appropriate media free from carbon sources other than the ones under study. Growth was evaluated by regular (every 30 min) measurement of optical density at 600 nm in an anaerobic environment (80% N₂, 10% CO₂, 10% H₂). Growth was expressed as area under the curve and compared to a positive control (glucose) and a negative control (no added carbon source).

Stock solutions of oligosaccharide soluble fibers (10% w/w) were prepared in demineralised water. The solutions were either sterilised by UV radiation or filtration (0.2 μm). Stocks were stored frozen until used. Appropriate carbon source-free medium was prepared from single ingredients. Test organisms were pre-grown anaerobically in the test medium with the standard carbon source. In honeycomb wells, 20 μL of stock solution was pipetted and 180 μL carbon source-free medium with 1% test microbe was added. As positive control, glucose was used as carbon source, and as negative control, no carbon source was used. To confirm sterility of the stock solutions, uninocculated wells were used. At least three parallel wells were used per run.

The honeycomb plates were placed in a Bioscreen and growth was determined by measuring absorbance at 600 nm. Measurements were taken every 30 min and before measurements, the plates were shaken to assure an even suspension of the microbes. Growth was followed for 24 h. Results were calculated as area under the curve (i.e., OD₆₀₀/24 h). Organisms tested (and their respective growth medium) were: Clostridium perfringens ATCC® 3626™ (anaerobic Reinforced Clostridial Medium (from Oxoid Microbiology Products, ThermoScientific) without glucose), Clostridium difficile DSM 1296 (Deutsche Sammlung von Mikroorganismen and Zellkulturen DSMZ, Braunschweig, Germany) (anaerobic Reinforced Clostridial Medium (from Oxoid Microbiology Products, Thermo Fisher Scientific Inc., Waltham, Mass.) without glucose), Escherichia coli ATCC® 11775™ (anaerobic Trypticase Soy Broth without glucose), Salmonella typhimurium EELA (available from DSMZ, Brauchschweig, Germany) (anaerobic Trypticase Soy Broth without glucose), Lactobacillus acidophilus NCFM 145 (anaerobic de Man, Rogosa and Sharpe Medium (from DSMZ) without glucose), Bifidobacterium animalis subsp. Lactis Bi-07 (anaerobic Deutsche Sammlung vom Mikroorgnismen and Zellkulturen medium 58 (from DSMZ), without glucose).

### In Vitro Gas Production

To measure the formation of gas by the intestinal microbiota, a pre-conditioned faecal slurry was incubated with test prebiotic (oligosaccharide or polysaccharide soluble fibers) and the volume of gas formed was measured. Fresh faecal material was pre-conditioned by dilution with 3 parts (w/v) of anaerobic simulator medium, stirring for 1 h under anaerobic conditions and filtering through 0.3-mm metal mesh after which it was incubated anaerobically for 24 h at 37° C.

The simulator medium used was composed as described by G. T. Macfarlane et al. (Microb. Ecol. 35(2):180-7 (1998)) containing the following constituents (g/L) in distilled water: starch (BDH Ltd.), 5.0; peptone, 0.05; tryptone, 5.0; yeast extract, 5.0; NaCl, 4.5; KCl, 4.5; mucin (porcine gastric type III), 4.0; casein (BDH Ltd.), 3.0; pectin (citrus), 2.0; xylan (oatspelt), 2.0; arabinogalactan (larch wood), 2.0; NaHCO₃, 1.5; MgSO₄, 1.25; guar gum, 1.0; inulin, 1.0; cysteine, 0.8; KH₂PO₄, 0.5; K₂HPO₄, 0.5; bile salts No. 3, 0.4; CaCl₂×6 H₂O, 0.15; FeSO₄×7 H₂O, 0.005; hemin, 0.05; and Tween 80, 1.0; cysteine hydrochloride, 6.3; Na₂S×9 H₂O, and 0.1% resazurin as an indication of sustained anaerobic conditions. The simulation medium was filtered through 0.3 mm metal mesh and divided into sealed serum bottles.

Test prebiotics were added from 10% (w/w) stock solutions to a final concentration of 1%. The incubation was performed at 37° C. while maintaining anaerobic conditions. Gas production due to microbial activity was measured manually after 24 h incubation using a scaled, airtight glass syringe, thereby also releasing the overpressure from the simulation unit.

### Example 1

### Construction of Glucosyltransferase (GTF-J) Expression Strain E. Coli MG1655/pMP52

The polynucleotide sequence encoding the mature glucosyltransferase enzyme (gtf-J; EC 2.4.1.5; SEQ ID NO: 3) from Streptococcus salivarius (ATCC® 25975™) as reported in GENBANK® (accession M64111.1; gi:47527) was synthesized using codons optimized for expression in E. coli (DNA 2.0, Menlo Park, Calif.). The nucleic acid product (SEQ ID NO: 4) encoding the mature enzyme (i.e., signal peptide removed and a start codon added; SEQ ID NO: 5) was subcloned into PJEXPRESS404® (DNA 2.0, Menlo Park Calif.) to generate the plasmid identified as pMP52. The plasmid pMP52 was used to transform E. coli MG1655 (ATCC® 47076™) to generate the strain identified as MG1655/pMP52. All procedures used for construction of the glucosyltransferase enzyme expression strain are well known in the art and can be performed by individuals skilled in the relevant art without undue experimentation.

### Example 2

### Production of Recombinant GTF-J in Fermentation

Production of the recombinant mature glucosyltransferase Gtf-J in a fermentor was initiated by preparing a pre-seed culture of the E. coli strain MG1655/pMP52, expressing the mature Gtf-J enzyme (GI:47527; “GTF7527”; SEQ ID NO: 5), constructed as described in Example 1. A 10-mL aliquot of the seed medium was added into a 125-mL disposable baffled flask and was inoculated with a 1.0 mL culture of E. coli MG1655/pMP52 in 20% glycerol. This culture was allowed to grow at 37° C. while shaking at 300 rpm for 3 h.

A seed culture for starting the fermentor was prepared by charging a 2-L shake flask with 0.5 L of the seed medium. 1.0 mL of the pre-seed culture was aseptically transferred into 0.5 L seed medium in the flask and cultivated at 37° C. and 300 rpm for 5 h. The seed culture was transferred at optical density>2 (OD₅₅₀) to a 14-L fermentor (Braun, Perth Amboy, N.J.) containing 8 L of the fermentor medium described above at 37° C.

Cells of E. coli MG1655/pMP52 were allowed to grow in the fermentor and glucose feed (50% w/w glucose solution containing 1% w/w MgSO₄.7H₂O) was initiated when glucose concentration in the medium decreased to 0.5 g/L. The feed was started at 0.36 grams feed per minute (g feed/min) and increased progressively each hour to 0.42, 0.49, 0.57, 0.66, 0.77, 0.90, 1.04, 1.21, 1.41 1.63, 1.92, 2.2 g feed/min respectively. The rate remained constant afterwards. Glucose concentration in the medium was monitored using an YSI glucose analyzer (YSI, Yellow Springs, Ohio). When glucose concentration exceeded 0.1 g/L the feed rate was decreased or stopped temporarily. Induction of glucosyltransferase enzyme activity was initiated, when cells reached an OD₅₅₀ of 70, with the addition of 9 mL of 0.5 M IPTG (isopropyl β-D-1-thiogalacto-pyranoside). The dissolved oxygen (DO) concentration was controlled at 25% of air saturation. The DO was controlled first by impeller agitation rate (400 to 1200 rpm) and later by aeration rate (2 to 10 standard liters per minute, slpm). The pH was controlled at 6.8. NH₄OH (14.5% weight/volume, w/v) and H₂SO₄ (20% w/v) were used for pH control. The back pressure was maintained at 0.5 bar. At various intervals (20, 25 and 30 hours), 5 mL of Suppressor 7153 antifoam (Cognis Corporation, Cincinnati, Ohio) was added into the fermentor to suppress foaming. Cells were harvested by centrifugation 8 h post IPTG addition and were stored at −80° C. as a cell paste.

### Example 3

### Preparation of GTF-J Crude Protein Extract from Cell Paste

The cell paste obtained as described in Example 2 was suspended at 150 g/L in 50 mM potassium phosphate buffer (pH 7.2) to prepare a slurry. The slurry was homogenized at 12,000 psi (˜82.7 MPa; Rannie-type machine, APV-1000 or APV 16.56; SPX Corp., Charlotte, N.C.) and the homogenate chilled to 4° C. With moderately vigorous stirring, 50 g of a floc solution (Aldrich no. 409138, 5% in 50 mM sodium phosphate buffer pH 7.0) was added per liter of cell homogenate. Agitation was reduced to light stirring for 15 minutes. The cell homogenate was then clarified by centrifugation at 4500 rpm for 3 hours at 5-10° C. Supernatant, containing Gtf-J enzyme in the crude protein extract, was concentrated (approximately 5×) with a 30 kilodalton (kDa) cut-off membrane. The concentration of total soluble protein in the Gtf-J crude protein extract was determined to be 4-8 g/L using the bicinchoninic acid (BCA) protein assay (Sigma Aldrich).

### Example 4

### Production of GTF-J GI:47527 in E. Coli TOP10

The plasmid pMP52 (Example 1) was used to transform E. coli TOP10 (Life Technologies Corp., Carlsbad, Calif.) to generate the strain identified as TOP10/pMP52. Growth of the E. coli strain TOP10/pMP52 expressing the mature Gtf-J enzyme “GTF7527” (provided as SEQ ID NO: 5) and determination of the GTF activity followed the methods described above.

### Example 5

### Production of GTF-L GI:662379 in E. Coli TOP10

A polynucleotide encoding a truncated version of a glucosyltransferase (Gtf) enzyme identified in GENBANK® as GI:662379 (SEQ ID NO: 6; Gtf-L from Streptococcus salivarius) was synthesized using codons optimized for expression in E. coli (DNA 2.0, Menlo Park Calif.). The nucleic acid product (SEQ ID NO: 7) encoding protein “GTF2379” (SEQ ID NO: 8), was subcloned into PJEXPRESS404® (DNA 2.0) to generate the plasmid identified as pMP65. The plasmid pMP65 was used to transform E. coli TOP10 (Life Technologies Corp.) to generate the strain identified as TOP10/pMP65. Growth of the E. coli strain TOP10/pMP65 expressing the gtf enzyme “2379” (last 4 digits of the respective GI number used) and determination of the Gtf activity followed the methods described above.

### Example 6

### Production of GTF-B GI:290580544 in E. Coli TOP10

A polynucleotide encoding a truncated version of a glucosyltransferase enzyme identified in GENBANK® as GI:290580544 (SEQ ID NO: 9; Gtf-B from Streptococcus mutans NN2025) was synthesized using codons optimized for expression in E. coli (DNA 2.0). The nucleic acid product (SEQ ID NO: 10) encoding protein “GTF0544” (SEQ ID NO: 11) was subcloned into PJEXPRESS404® to generate the plasmid identified as pMP67. The plasmid pMP67 was used to transform E. coli TOP10 to generate the strain identified as TOP10/pMP67. Growth of the E. coli strain TOP10/pMP67 expressing the Gtf-B enzyme “GTF0544” (SEQ ID NO: 11) and determination of the GTF0544 activity followed the methods described above.

### Example 7

### Production of GTF-I GI:450874 in E. Coli BL21 DE3

A polynucleotide encoding a glucosyltransferase from Streptococcus sobrinus, (ATCC® 27351™) was isolated using polymerase chain reaction (PCR) methods well known in the art. PCR primers were designed based on gene sequence described in GENBANK® accession number BAA14241 and by Abo et al., (J. Bacteriol., (1991) 173:998-996). The 5′-end primer 5′-GGGAATTCCCAGGTTGACGGTAAATATTATTACT-3′ (SEQ ID NO: 12) was designed to code for sequence corresponded to bases 466 through 491 of the gtf-I gene. Additionally the primer contained sequence for an EcoRI restriction enzyme site which was used for cloning purposes.

The 3′-End Primer

5′-AGATCTAGTCTTAGTTCCAGCCACGGTACATA-3′ (SEQ ID NO: 13) was designed to code for sequence corresponded to the reverse compliment of bases 4749 through 4774 of S. sobrinus gene. The reverse PCR primer also included the sequence for an XbaI site, used for cloning purposes. The resulting 4.31 Kb DNA fragment was digested with EcoRI and Xba I restriction enzymes and purified using a Promega PCR Clean-up kit (A9281, Promega Corp., Madison, Wis.) as recommended by the manufacturer. The DNA fragment was ligated into an E. coli protein expression vector (pET24a, Novagen, a divisional of Merck KGaA, Darmstadt, Germany). The ligated reaction was transformed into the BL21 DE3 cell line (New England Biolabs, Ipswich, Mass.) and plated on solid LB medium (10 g/L, tryptone; 5 g/L yeast extract; 10 g/L NaCl; 14% agar; 100 μg/mL ampicillin) for selection of single colonies.

Transformed E. coli BL21 DE3 cells were inoculated to an initial optical density (OD at 600ₙₘ) of 0.025 in LB media and were allowed to grow at 37° C. in an incubator while shaking at 250 rpm. When cultures reached an OD of 0.8-1.0, the gene (SEQ ID NO: 15) encoding the truncated Gtf-I enzyme (SEQ ID NO: 16) was induced by addition of 1 mM IPTG. Induced cultures remained on the shaker and were harvested 3 h post induction. Cells were harvested by centrifugation (25° C., 16,000 rpm) using an Eppendorf centrifuge. Cell pellets were suspended at 0.01 volume in 5.0 mM phosphate buffer (pH 7.0) and cooled to 4° C. on ice. The cells were broken using a bead beater with 0.1 millimeters (mm) silica beads. Cell debris was removed by centrifuged (16,000 rpm for 10 minutes at 4° C.). The crude protein extract (containing soluble Gtf-I (“GTF0874”) enzyme) was aliquoted and stored at −80° C.

### Example 8

### Production of GTF-I Enzyme GI:450874 in E. Coli TOP10

The gene encoding a truncated version of a glucosyltransferase enzyme identified in GENBANK® as GI:450874 (SEQ ID NO: 14; Gtf-I from Streptococcus sobrinus) was synthesized using codons optimized for expression in E. coli (DNA 2.0). The nucleic acid product (SEQ ID NO: 15) encoding the truncated glucosyltransferase (“GTF0874”; SEQ ID NO: 16) was subcloned into PJEXPRESS404® to generate the plasmid identified as pMP53. The plasmid pMP53 was used to transform E. coli TOP10 to generate the strain identified as TOP10/pMP53. Growth of the E. coli strain TOP10/pMP53 expressing the Gtf-I enzyme “GTF0874” and determination of Gtf activity followed the methods described above.

### Example 9

### Production of GTF-S Enzyme GI: 495810459 in E. Coli TOP10

A gene encoding a truncated version of a glucosyltransferase enzyme identified in GENBANK® as GI:495810459 (SEQ ID NO: 17; Gtf-S from Streptococcus sp. C150) was synthesized using codons optimized for expression in E. coli (DNA 2.0). The nucleic acid product (SEQ ID NO: 18) encoding the truncated glucosyltransferase (“GTF0459”; SEQ ID NO: 19) was subcloned into PJEXPRESS404® to generate the plasmid identified as pMP79. The plasmid pMP79 was used to transform E. coli TOP10 to generate the strain identified as TOP10/pMP79. Growth of the E. coli strain TOP10/pMP79 expressing the Gtf-S enzyme and determination of the Gtf activity followed the methods described above.

### Example 10

### Production of GTF-S Enzyme GI: 495810459 in B. Subtilis BG6006

SG1067-2 is a Bacillus subtilis expression strain that expresses a truncated version of the glycosyltransferase Gtf-S (“GTF0459”) from Streptococcus sp. C150 (GI:495810459). The B. subtilis host BG6006 strain contains 9 protease deletions (amyE::xylRPxylAcomK-ermC, degUHy32, oppA, ΔspoIIE3501, ΔaprE, ΔnprE, Δepr, ΔispA, Δbpr, Δvpr, ΔwprA, Δmpr-ybfJ, ΔnprB). The full length Gtf-A has 1570 amino acids. The N terminal truncated version with 1393 amino acids was originally codon optimized for E. coli expression and synthesized by DNA2.0. This N terminal truncated Gtf-S (SEQ ID NO: 19) was subcloned into the NheI and HindIII sites of the replicative Bacillus expression pHYT vector under the aprE promoter and fused with the B. subtilis AprE signal peptide on the vector. The construct was first transformed into E. coli DH10B and selected on LB with ampicillin (100 μg/mL) plates. The confirmed construct pDCQ967 expressing the Gtf was then transformed into B. subtilis BG6006 and selected on the LB plates with tetracycline (12.5 μg/mL). The resulting B. subtilis expression strain SG1067 was purified and one of isolated cultures, SG1067-2, was used as the source of the Gtf-S enzyme. SG1067-2 strain was first grown in LB media containing 10 μg/mL tetracycline, and then subcultured into GrantsII medium containing 12.5 μg/mL tetracycline grown at 37° C. for 2-3 days. The cultures were spun at 15,000 g for 30 min at 4° C. and the supernatant was filtered through 0.22 μm filters. The filtered supernatant containing GTF0459 was aliquoted and frozen at −80° C.

### Example 11

### Fermentation of B. Subtilis SG1067-2 to Produce GTF-S GI:495810459

B. subtilis SG1067-2 strain (Example 10), expressing GTF0459 (SEQ ID NO: 19), was grown under an aerobic submerged condition by conventional fed-batch fermentation. A nutrient medium contains 0-15% HY-SOY™ (a highly soluble, multi-purpose, enzymatic hydrolysate of soy meal; Kerry Inc., Beloit, Wis.), 5-25 g/L sodium and potassium phosphate, 0.5-4 g/L magnesium sulfate, and citric acid, ferrous sulfate and manganese sulfate. An antifoam agent, FOAM BLAST® 882 (a food grade polyether polyol defoamer aid; Emerald Performance Materials, LLC, Cuyahoga Falls, Ohio), of 3-5 mL/L was added to control foaming. 2-L fermentation was fed with 50% w/w glucose feed when initial glucose in batch was non-detectable. The glucose feed rate was ramped over several hours. The fermentation was controlled at 37° C. and 20% DO, and initiated at the initial agitation of 400 rpm. The pH was controlled at 7.2 using 50% v/v ammonium hydroxide. Fermentation parameters such as pH, temperature, airflow, DO % were monitored throughout the entire 2-day fermentation run. The culture broth was harvested at the end of run and centrifuged at 5° C. to obtain supernatant. The supernatant containing GTF0459 was then frozen and stored at −80° C.

### Example 11A

### Construction of Bacillus Subtilis Strains Expressing Homolog Genes of GTF0459

A search was carried out to identify sequences homologous to GTF0459. Beginning with the GTF0459 sequence, homologous sequences were identified by carrying out a BLAST search against the non-redundant NCBI protein database as of Sep. 8, 2014. The BLAST run identified about 1100 putative homologs using an e-value cutoff of 1e-10. After filtering for alignments of at least 1000 amino acids in length and sorting based on percentage amino acid sequence identity, 13 homologs were found which were closely related, i.e., had greater than 90% amino acid sequence identity, to GTF0459. The identified homologs were then aligned to the GTF0459 sequence by using CLUSTALW, a standard sequence alignment package for aligning very highly related sequences. The homologous sequences are around 96-97% identitical to the amino acid sequence of GTF0459 in the aligned region of 1570 residues. The aligned region extends from amino acid position 1 to 1570 in GTF0459 and positions 1 to 1581 in the GTF0459 homologs. Beyond the 13 identified GTF0459 homologs, the next closest proteins share only about 55% amino acid sequence identity in the aligned region to GTF0459 or any of the 13 identified homologs. The DNA sequences encoding N terminal variable region truncated proteins of GTF0459 and the homologs (SEQ ID NOs. 86 and the odd numbered SEQ ID NOs between 87 and 111) and two non-homologs (<54% aa sequence identity) (SEQ ID NOs. 113, 115) as provided in the table 1 below were synthesized by Genscript. The synthetic genes were cloned into the NheI and HindIII sites of the Bacillus subtilis integrative expression plasmid p4JH under the aprE promoter and fused with the B. subtilis AprE signal peptide on the vector. In some cases, they were cloned into the SpeI and HindIII sites of the Bacillus subtilis integrative expression plasmid p4JH under the aprE promoter without a signal peptide. The constructs were first transformed into E. coli DH10B and selected on LB with ampicillin (100 ug/ml) plates. The confirmed constructs expressing the particular GTFs were then transformed into B. subtilis host containing 9 protease deletions (amyE::xylRPxylAcomK-ermC, degUHy32, oppA, ΔspoIIE3501, ΔaprE, ΔnprE, Δepr, ΔispA, Δbpr, Δvpr, ΔwprA, Δmpr-ybfJ, ΔnprB) and selected on the LB plates with chloramphenicol (5 ug/ml). The colonies grown on LB plates with 5 ug/ml chloramphenicol were streaked several times onto LB plates with 25 ug/ml chloramphenicol. The resulting B. subtilis expression strains were grown in LB medium with 5 ug/ml chloramphenicol first and then subcultured into GrantsII medium grown at 30° C. for 2-3 days. The cultures were spun at 15,000 g for 30 min at 4° C. and the supernatants were filtered through 0.22 um filters. The filtered supernatants were aliquoted and frozen at −80° C.

TABLE 1
GTF0459 and sequences identified during homolog search (GTF
numbering based on last four digits of GI number)

| | | | | DNA | aa |
|---|---|---|---|---|---|
| | | | | seq | seq |
| | New GI | % | | SEQ | SEQ |
| GI number | number | identity | Source organisms | ID | ID |
| 322373279 | 495810459; | 100.00 | Streptococcus sp. C150 | 86 | 19 |
| | 321278321 | | | | |
| 488980470 | | 97.41 | Streptococcus salivarius K12 | 87 | 88 |
| 488977317 | | 97.56 | Streptococcus salivarius PS4 | 89 | 90 |
| 544721645 | | 97.13 | Streptococcus sp. HSISS3 | 91 | 92 |
| 544716099 | | 97.27 | Streptococcus sp. HSISS2 | 93 | 94 |
| 660358467 | | 96.98 | Streptococcus salivarius NU10 | 95 | 96 |
| 340398487 | 503756246 | 96.77 | Streptococcus salivarius CCHSS3 | 97 | 98 |
| 490286549 | | 96.41 | Streptococcus salivarius M18 | 99 | 100 |
| 544713879 | | 96.62 | Streptococcus sp. HSISS4 | 101 | 102 |
| 488974336 | | 96.77 | Streptococcus salivarius SK126 | 103 | 104 |
| 387784491 | 504447649 | 96.34 | Streptococcus salivarius JIM8777 | 105 | 106 |
| 573493808 | | 96.26 | Streptococcus sp. SR4 | 107 | 108 |
| 387760974 | 504445794 | 96.12 | Streptococcus salivarius 57.I | 109 | 110 |
| 576980060 | | 96.12 | Streptococcus sp. ACS2 | 111 | 112 |
| 495810487 | | 53 | Streptococcus salivarius PS4 | 113 | 114 |
| 440355360 | | 48.02 | Streptococcus mutans JP9-4 | 115 | 116 |

### Example 11B

### Construction of Bacillus Subtilis Strains Expressing C-Terminal Truncations of GTF0459 Homolog Genes

Glucosyltransferases usually contain an N terminal variable domain, a middle catalytic domain, and a C-terminal domain containing multiple glucan-binding domains. The GTF0459 homologs identified and expressed in Example 11A all contained an N terminal variable region truncation. This example describes the construction of Bacillus subtilis strains expressing individual C-terminal truncations of GTF0459 and GTF0459 homologs (as identified by the last four digits in the GI numbers in table 1 above).

T1 (extending from amino acid positions 179-1086), T2 (extending from amino acid positions 179-1125), T4 (extending from amino acid positions 179-1182), T5 (extending from amino acid positions 179-1183), and T6 (extending from amino acid positions 179-1191) C-terminal truncations were made from the GTF0974, GTF4336, and GTF4491 glucosyltransferases containing N-terminal trunctations as listed in table 1 in Example 11A. A T5 and T6 truncation of GTF0459 (GTF3279) was also produced. A T5 truncation was also made from GTF3808. DNA and protein SEQ ID NOs for the sequences of the truncations as provided in the sequence listing are listed in table 2 below. The DNA fragments encoding GTF0459, the N-terminal truncated homologs, and the C-terminal truncations were PCR amplified from the synthetic gene plasmids by Genscript and cloned into the SpeI and HindIII sites of the Bacillus subtilis integrative expression plasmid p4JH under the aprE promoter without a signal peptide. The constructs were first transformed into E. coli DH10B and selected on LB with ampicillin (100 ug/ml) plates. The confirmed constructs expressing the particular GTFs were then transformed into B. subtilis host containing 9 protease deletions (amyE::xylRPxylAcomK-ermC, degUHy32, oppA, ΔspoIIE3501, ΔaprE, ΔnprE, Δepr, ΔispA, Δbpr, Δvpr, ΔwprA, Δmpr-ybfJ, ΔnprB) and selected on the LB plates with chloramphenicol (CM, 5 ug/ml). The colonies grown on LB plates with 5 ug/ml chloramphenicol were streaked several times onto LB plates with 25 ug/ml chloramphenicol. The resulting B. subtilis expression strains were grown in LB medium with 5 ug/ml chloramphenicol first and then subcultured into GrantsII medium grown at 30° C. for 2-3 days. The cultures were spun at 15,000 g for 30 min at 4° C. and the supernatants were filtered through 0.22 um filters. The filtered supernatants were aliquoted and frozen at −80° C.

GTF activity of the strains was analyzed by PAHBAH assay in three separate experiments. Due to minor variations between the expeirments, Table 2 lists the activity of the truncated enzymes in the B. subtilis host along with the experiment in which the activity was measured. Most of the T1, T2, and T6 truncations decreased the activity of the enzymes, whereas the T4 and T5 C-terminal truncations retained similar activity relative to the respective N terminal-only truncations (NT). The homologs and C-terminal truncations of the homologs maintained activity and produced a similar soluble α-glucan fiber to GTF0459 (see Examples 39A and 39B), suggesting that residues within the catalytic domain retained in the truncations may be a characteristic of enzymes capable of producing the fiber. To identify specific amino acid residues within the catalytic domain that may be involved in producing the soluble α-glucan fiber, we analyzed the crystal structures (PDB Identifiers: 3AIB, 3AIC, and 3HZ3) of the catalytic domains of three glucosyltransferases to identify residues within 8 Angstroms of the bound ligand. 57 residues met that criterion. A motif was generated based on the corresponding 57 amino acids in GTF0459 and each of the identified homologs. The motif was then used to generate a consensus sequence to capture the variability in the catalytic domains of GTF0459 and the identified homologs. The consensus sequence is provided as SEQ ID NO: 153.

TABLE 2
GTF activity of strains.

| | | | | DNA | Amino |
|---|---|---|---|---|---|
| | | Experiment | Acitivity, | SEQ ID | Acid SEQ |
| Strain | Enzyme | Number | U/mL | NO: | ID NO: |
| SG1316 | GTF0974T4 | 2 | 47.2 | 127 | 128 |
| SG1316 | GTF0974T4 | 3 | 33.9 | 127 | 128 |
| SG1317 | GTF0974T5 | 2 | 43.5 | 117 | 118 |
| SG1317 | GTF0974T5 | 3 | 37.7 | 117 | 118 |
| SG1290 | GTF0974NT | 1 | 43.7 | 109 | 110 |
| SG1290 | GTF0974NT | 2 | 53 | 109 | 110 |
| SG1290 | GTF0974NT | 3 | 36.4 | 109 | 110 |
| SG1318 | GTF4336T4 | 2 | 46.4 | 129 | 130 |
| SG1319 | GTF4336T5 | 2 | 43.6 | 119 | 120 |
| SG1291 | GTF4336NT | 1 | 34.5 | 103 | 104 |
| SG1291 | GTF4336NT | 2 | 48.6 | 103 | 104 |
| SG1320 | GTF4491T4 | 2 | 45.3 | 131 | 132 |
| SG1321 | GTF4491T5 | 2 | 50.6 | 121 | 122 |
| SG1292 | GTF4491NT | 1 | 42.3 | 105 | 106 |
| SG1292 | GTF4491NT | 2 | 53.1 | 105 | 106 |
| SG1330 | GTF3808T5 | 3 | 36.2 | 123 | 124 |
| SG1313 | GTF3808NT | 3 | 34.9 | 107 | 108 |
| SG1297 | GTF0459NTnativeT5 | 2 | 52 | 125 | 126 |
| SG1298 | GTF0459NTnativeT6 | 1 | 28.5 | 133 | 134 |
| SG1273 | GTF0459nativeNT | 1 | 26.5 | 86 | 19 |
| SG1273 | GTF0459nativeNT | 2 | 39.4 | 86 | 19 |
| SG1304 | GTF0974T1 | 1 | 18.4 | 135 | 136 |
| SG1305 | GTF0974T2 | 1 | 7.2 | 137 | 138 |
| SG1306 | GTF0974T6 | 1 | 33.7 | 139 | 140 |
| SG1307 | GTF4336T1 | 1 | 9.4 | 141 | 142 |
| SG1308 | GTF4336T2 | 1 | 11.5 | 143 | 144 |
| SG1309 | GTF4336T6 | 1 | 28.9 | 145 | 146 |
| SG1310 | GTF4991T1 | 1 | 23.1 | 147 | 148 |
| SG1311 | GTF4991T2 | 1 | 4.9 | 149 | 150 |
| SG1312 | GTF4991T6 | 1 | 1.7 | 151 | 152 |

### Example 11C

### Fermentation of Bacillus Subtilis Strains Expressing Homologs of GTF0459 or C-Terminal Truncations of GTF0459 Homologs using Soy Hydrolysate Medium

A B. subtilis strain expressing each GTF was grown under an aerobic submerged condition by conventional fed-batch fermentation. The nutrient medium contained 1.75-7% soy hydrolysate (Sensient or BD), 5-25 g/L sodium and potassium phosphate, 0.5-4 g/L magnesium sulfate and a solution of 3-10 g/L citric acid, ferrous sulfate and manganese. An antifoam agent, Foamblast 882, at 2-4 mL/L was added to control foaming. A 2-L or 10-L fermentation was fed with 50% w/w glucose feed when initial glucose in batch was non-detectable. The glucose feed rate was ramped over several hours. The fermentation was controlled at 20% DO and temperature of 30° C., and initiated at an initial agitation of 400 rpm. The pH was controlled at 7.2 using 50% v/v ammonium hydroxide. Fermentation parameters such as pH, temperature, airflow, DO % were monitored throughout the entire 2-3 day fermentation run. The culture broth was harvested at the end of run and centrifuged to obtain supernatant containing GTF. The supernatant was then stored frozen at −80° C.

### Example 11D

### Fermentation of Bacillus Subtilis Strains Expressing Homologs of GTF0459 or C-Terminal Truncations of GTF0459 Homologs using Corn Steep Solids Medium

A B. subtilis strain expressing each GTF was grown under an aerobic submerged condition by conventional fed-batch fermentation. A nutrient medium contained 0.5-2.5% corn steep solids (Roquette), 5-25 g/L sodium and potassium phosphate, a solution of 0.3-0.6 M ferrous sulfate, manganese chloride and calcium chloride, 0.5-4 g/L magnesium sulfate, and a solution of 0.01-3.7 g/L zinc sulfate, cuprous sulfate, boric acid and citric acid. An antifoam agent, Foamblast 882, of 2-4 mL/L was added to control foaming. 2-L fermentation was fed with 50% w/w glucose feed when initial glucose in batch was non-detectable. The glucose feed rate was ramped over several hours. The fermentation was controlled at 20% DO and temperature of either 30° C. or 37° C., and initiated at an initial agitation of 400 rpm. The pH was controlled at 7.2 using 50% v/v ammonium hydroxide. Fermentation parameters such as pH, temperature, airflow, DO % were monitored throughout the entire 2-3 day fermentation run. The culture broth was harvested at the end of run and centrifuged to obtain supernatant containing GTF. The supernatant was then stored frozen at −80° C.

### Example 12

### Production of Mutanase MUT3264 GI: 257153264 in E. Coli BL21(DE3)

A gene encoding mutanase from Paenibacillus humicus NA1123 identified in GENBANK® as GI:257153264 (SEQ ID NO: 22) was synthesized by GenScript (GenScript USA Inc., Piscataway, N.J.). The nucleotide sequence (SEQ ID NO: 20) encoding protein sequence (“MUT3264”; SEQ ID NO: 21) was subcloned into pET24a (Novagen; Merck KGaA, Darmstadt, Germany). The resulting plasmid was transformed into E. coli BL21(DE3) (Invitrogen) to generate the strain identified as SGZY6. The strain was grown at 37° C. with shaking at 220 rpm to OD₆₀₀ of ˜0.7, then the temperature was lowered to 18° C. and IPTG was added to a final concentration of 0.4 mM. The culture was grown overnight before harvest by centrifugation at 4000 g. The cell pellet from 600 mL of culture was suspended in 22 mL 50 mM KPi buffer, pH 7.0. Cells were disrupted by French Cell Press (2 passages @ 15,000 psi (103.4 MPa)); cell debris was removed by centrifugation (SORVALL™ SS34 rotor, @13,000 rpm; Thermo Fisher Scientific, Inc., Waltham, Mass.) for 40 min. The supernatant was analyzed by SDS-PAGE to confirm the expression of the “mut3264” mutanase and the crude extract was used for activity assay. A control strain without the mutanase gene was created by transforming E. coli BL21(DE3) cells with the pET24a vector.

### Example 13

### Production of Mutanase MUT3264 GI: 257153264 in B. Subtilis Strain BG6006 Strain SG1021-1

SG1021-1 is a Bacillus subtilis mutanase expression strain that expresses the mutanase from Paenibacillus humicus NA1123 isolated from fermented soy bean natto. For recombinant expression in B. subtilis, the native signal peptide was replaced with a Bacillus AprE signal peptide (GENBANK® Accession No. AFG28208; SEQ ID NO: 25). The polynucleotide encoding MUT3264 (SEQ ID NO: 23) was operably linked downstream of an AprE signal peptide (SEQ ID NO: 25) encoding Bacillus expressed MUT3264 provided as SEQ ID NO: 24. A C-terminal lysine was deleted to provide a stop codon prior to a sequence encoding a poly histidine tag.

The B. subtilis host BG6006 strain contains 9 protease deletions (amyE::xylRPxylAcomK-ermC, degUHy32, oppA, ΔspoIIE3501, ΔaprE, ΔnprE, Δepr, ΔispA, Δbpr, Δvpr, ΔwprA, Δmpr-ybfJ, ΔnprB). The wild type mut3264 (as found under GENBANK® GI: 257153264) has 1146 amino acids with the N terminal 33 amino acids deduced as the native signal peptide by the SignalP 4.0 program (Nordahl et al., (2011) Nature Methods, 8:785-786). The mature mut3264 without the native signal peptide was synthesized by GenScript and cloned into the NheI and HindIII sites of the replicative Bacillus expression pHYT vector under the aprE promoter and fused with the B. subtilis AprE signal peptide (SEQ ID NO: 25) on the vector. The construct was first transformed into E. coli DH10B and selected on LB with ampicillin (100 μg/mL) plates. The confirmed construct pDCQ921 was then transformed into B. subtilis BG6006 and selected on the LB plates with tetracycline (12.5 μg/mL). The resulting B. subtilis expression strain SG1021 was purified and a single colony isolate, SG1021-1, was used as the source of the mutanase mut3264. SG1021-1 strain was first grown in LB containing 10 μg/mL tetracycline, and then sub-cultured into GrantsII medium containing 12.5 μg/mL tetracycline and grown at 37° C. for 2-3 days. The cultures were spun at 15,000 g for 30 min at 4° C. and the supernatant filtered through a 0.22 μm filter. The filtered supernatant containing MUT3264 was aliquoted and frozen at −80° C.

### Example 14

### Production of Mutanase MUT3325 GI: 212533325

A gene encoding the Penicillium marneffei ATCC® 18224™ mutanase identified in GENBANK® as GI:212533325 was synthesized by GenScript (Piscataway, N.J.). The nucleotide sequence (SEQ ID NO: 26) encoding protein sequence (MUT3325; SEQ ID NO: 27) was subcloned into plasmid pTrex3 (SEQ ID NO: 59) at SacII and AscI restriction sites, a vector designed to express the gene of interest in Trichoderma reesei, under control of CBHI promoter and terminator, with Aspergillus niger acetamidase for selection. The resulting plasmid was transformed into T. reesei by biolistic injection as described in the general method section, above. The detailed method of biolistic transformation is described in International PCT Patent Application Publication WO2009/126773 A1. A 1 cm² agar plug with spores from a stable clone TRM05-3 was used to inoculate the production media (described below). The culture was grown in the shake flasks for 4-5 days at 28° C. and 220 rpm. To harvest the secreted proteins, the cell mass was first removed by centrifugation at 4000 g for 10 min and the supernatant was filtered through 0.2 μM sterile filters. The expression of mutanase MUT3325 was confirmed by SDS-PAGE. The production media components are listed below.

### NREL-Trich Lactose Defined

| Formula | Amount | Units |
|---|---|---|
| ammonium sulfate | 5 | g |
| PIPPS | 33 | g |
| BD Bacto casamino acid | 9 | g |
| KH₂PO₄ | 4.5 | g |
| CaCl₂•2H₂O | 1.32 | g |
| MgSO₄•7H₂O | 1 | g |
| T. reesei trace elements | 2.5 | mL |
| NaOH pellet | 4.25 | g |
| Adjust pH to 5.5 with 50% | | |
| NaOH | | |
| Bring volume to | 920 | mL |
| Add to each aliquot: | 5 | Drops |
| Foamblast | | |
| Autoclave, then add | 80 | mL |
| 20% lactose filter sterilized | | |

### T. Reesei Trace Elements

| Formula | Amount | Units |
|---|---|---|
| citric acid•H₂O | 191.41 | g |
| FeSO₄•7H₂O | 200 | g |
| ZnSO₄•7H₂O | 16 | g |
| CuSO₄•5H₂O | 3.2 | g |
| MnSO₄•H₂O | 1.4 | g |
| H₃BO₃ (boric acid) | 0.8 | g |
| Bring volume to | 1 | L |

### Example 15

### Production of MUT3325 by Fermentation

Fermentation seed culture was prepared by inoculating 0.5 L of minimal medium in a 2-L baffled flask with 1.0 mL frozen spore suspension of the MUT3325 expression strain TRM05-3 (Example 14) (The minimal medium was composed of 5 g/L ammonium sulfate, 4.5 g/L potassium phosphate monobasic, 1.0 g/L magnesium sulfate heptahydrate, 14.4 g/L citric acid anhydrous, 1 g/L calcium chloride dihydrate, 25 g/L glucose and trace elements including 0.4375 g/L citric acid, 0.5 g/L ferrous sulfate heptahydrate,0.04 g/L zinc sulfate heptahydrate, 0.008 g/L cupric sulfate pentahydrate, 0.0035 g/L manganese sulfate monohydrate and 0.002 g/L boric acid. The pH was 5.5.). The culture was grown at 32° C. and 170 rpm for 48 hours before transferred to 8 L of the production medium in a 14-L fermentor. The production medium was composed of 75 g/L glucose, 4.5 g/L potassium phosphate monobasic, 0.6 g/L calcium chloride dehydrate, 1.0 g/L magnesium sulfate heptahydrate, 7.0 g/L ammonium sulfate, 0.5 g/L citric acid anhydrous, 0.5 g/L ferrous sulfate heptahydrate, 0.04 g/L zinc sulfate heptahydrate, 0.00175 g/L cupric sulfate pentahydrate, 0.0035 g/L manganese sulfate monohydrate, 0.002 g/L boric acid and 0.3 mL/L foam blast 882.

The fermentation was first run with batch growth on glucose at 34° C., 500 rpm for 24 h. At the end of 24 h, the temperature was lowered to 28° C. and agitation speed was increased to 1000 rpm. The fermentor was then fed with a mixture of glucose and sophorose (62% w/w) at specific feed rate of 0.030 g glucose-sophorose solids/g biomass/hr. At the end of run, the biomass was removed by centrifugation and the supernatant containing the mutanase was concentrated about 10-fold by ultrafiltration using 10-kD Molecular Weight Cut-Off ultrafiltration cartridge (UFP-10-E-35; GEHealthcare, Little Chalfont, Buckinghamshire, UK). The concentrated protein was stored at −80° C.

### Example 16

### Production of Mutanase MUT6505 (GI: 259486505)

A polynucleotide encoding the Aspergillus nidulans FGSC A4 mutanase identified in GENBANK® as GI:259486505 was synthesized by GenScript (Piscataway, N.J.). The nucleotide sequence (SEQ ID NO: 28) encoding protein sequence (MUT6505; SEQ ID NO: 29) was subcloned into plasmid pTrex3, a vector designed to express the gene of interest in T. reesei, under control of CBHI promoter and terminator, with A. niger acetamidase for selection. The resulting plasmid was transformed into T. reesei by biolistic injection. A 1 cm² agar plug with spores from a stable clone was used to inoculate the production media (ammonium sulfate 5 g/L, PIPPS 33 g/L; BD Bacto casamino acid 9 g/L, KH₂PO₄ 4.5 g/L, CaCl₂.2H₂O 1.32 g/L, MgSO₄.7H₂O 1 g/L, NaOH pellet 4.25 g/L, lactose 1.6 g/L, antifoam 204 0.01%, citric acid.H₂O 0.48 g/L, FeSO₄.7H₂O 0.5 g/L, ZnSO₄.7H₂O 0.04 g/L, CuSO₄.5H₂O 0.008 g/L, MnSO₄.H₂O 0.0036 g/L and boric acid 0.002 g/L at pH 5.5.). The culture was grown in the shake flasks for 4-5 days at 28° C. and 220 rpm. To harvest the secreted proteins, the cell mass was first removed by centrifugation at 4000 g for 10 min and the supernatant was filtered through 0.2 μM sterile filters. The expression of MUT6505 was confirmed by SDS-PAGE. The crude protein extract containing MUT6505 was stored at −80° C.

### Example 17

### Production of H. Tawa, T. Konilangbra and T. Reesei Mutanases

The following describes the methods used to obtain the respective polynucleotide and amino acid sequences for mutanases from Hypocrea tawa (SEQ ID NOs: 53 and 54), Trichoderma konilangbra (SEQ ID NOs: 55 and 56), and Trichoderma reesei (SEQ ID NOs: 57 and 58).

### Isolation of Genomic DNA

Fungal cultures of Trichoderma reesei 592, Trichoderma konilangbra and Hypocrea tawa were prepared (see EP2644187A1 and corresponding U.S. Patent Appl. Pub. No 2011-0223117A1 to Kim et al.) by adding 30 mL of sterile YEG broth to three 250-mL baffled Erlenmeyer shaking flasks in the biological hood. A 131-inch (˜333 cm) square was cut and removed from each respective fungal culture plate using a sterile plastic loop and placed into the appropriate culture flask. The inoculated flasks were then placed into the 28° C. shaking incubator to grow overnight.

The T. reesei, T. konilangbra, and H. tawa cultures were removed from the shaking incubator and the contents of each flask were poured into separate sterile 50 mL Sarstedt tubes. The Sarstedt tubes were placed in a table-top centrifuge and spun at 4,500 rpm for 10 min to pellet the fungal mycelia. The supernatants were discarded and a large loopful of each mycelial sample was transferred to a separate tube containing lysing matrix (FASTDNA™). Genomic DNA was extracted from the harvested mycelia using the FASTDNA™ kit (Qbiogene, now MP Biomedicals Inc., Santa Ana, Calif.) according to the manufacturer's protocol for algae, fungi and yeast. The homogenization time was 25 seconds. The amount and quality of genomic DNA extracted was determined by gel electrophoresis.

### Obtaining Alpha-Glucanase Polypeptides by PCR

### A. T. Reesei

Putative α-1,3 glucanase genes were identified in the T. reesei genome (JGI) by homology. PCR primers for T. reesei were designed based on the putative homolog DNA sequences. Degenerate PCR primers were designed for T. konilangbra or H. tawa based on the putative T. reesei protein sequences and other published α-1,3 glucanase protein sequences.

### T. Reesei Specific PCR Primers:

SK592:
(SEQ ID NO: 30)
5′-CACCATGTTTGGTCTTGTCCGC-3′
SK593:
(SEQ ID NO: 31)
5′-TCAGCAGTACTGGCATGCTG-3′

The PCR conditions used to amplify the putative α-1,3 glucanase from genomic DNA extracted from T. reesei strain RL-P37 (U.S. Pat. No. 4,797,361A; NRRL-15709, Agricultural Research Services, USDA, Peoria, Ill.) were as follows:

1. 94° C. for 2 minutes,

2. 94° C. tor 30 seconds,

3. 56° C. for 30 seconds,

4. 72° C. for 3 minutes,

5. return to step 2 for 24 cycles,

6. hold at 4° C.

Reaction samples contained 2 mL of T. reesei RL-P37 genomic DNA, 10 mL of the 10× buffer, 2 mL 10 mM dN TPs mixture, 1 mL primers SK592 and SK593 at 20 mM, 1 mL of the PfuUltra high fidelity DNA polymerase (Agilent Technologies, Santa Clara, Calif.) and 83 mL distillled water.

### B. T. Konilangbra and H. Tawa

Initial PCR reactions used degenerate primers designed from protein alignments of several homologous sequences. A primary set of degenerate primers, designed to anneal near the 5′ and 3′ ends, were used in the first PCR reaction to amplify similar sequences to that of an α-1,3 glucanase. Degenerate primers for initial cloning:

### H. Tawa and T. Konilangbra:

MA1F:
(SEQ ID NO: 32)
5′-GTNTTYTGYCAYTTYATGAT-3′
MA2F:
(SEQ ID NO: 33)
5′-GTNTTYTGYACAYTTYATGATHGGNAT-3′
MA4F:
(SEQ ID NO: 34)
5′-GAYTAYGAYGAYGAYATGCARCG-3′
MA5F:
(SEQ ID NO: 35)
5′-GTRCAYTTRCAIGGICCIGGIGGRCARTANCC-3′
MA6R:
(SEQ ID NO: 36)
5′-YTCICCIGGNAGNGGRCANCCRTT-3′
MA7R:
(SEQ ID NO: 37)
5′-RCARTAYTGRCAIGCYGTYGGYGGRCARTA-3′

The products of these PCR reactions were then used in a nested PCR using primers designed to attach within the product of the initial PCR fragment, under the same amplification conditions Specific primers for initial cloning:

### T. Konilangbra:

TP1S:
(SEQ ID NO: 38)
5′-CCCCCTGGCCAAGTATGTGT-3′
TP2A:
(SEQ ID NO: 39)
5′-GTACGCAAAGTTGAGCTGCT-3′
TP3S:
(SEQ ID NO: 40)
5′-AGCACATCGCTGATGGATAT-3′
TP3A:
(SEQ ID NO: 41)
5′-AAGTATACGTTGCTTCCGGC-3′
TP4S:
(SEQ ID NO: 42)
5′-CTGACGATCGGACTRCACGT-3′
TP4A:
(SEQ ID NO: 43)
5′-CGTTGTCGACGTAGAGCTGT-3′

### H. Tawa:

HP2A:
(SEQ ID NO: 44)
5′-ACGATCGGCAGAGTCATAGG-3′
HP3S:
(SEQ ID NO: 45)
5′-ATCGGATTGCATGTCACGAC-3′
HP3A:
(SEQ ID NO: 46)
5′-TACATCCAGACCGTCACCAG-3′
HP4S:
(SEQ ID NO: 47)
5′-ACGTTTGCTCTTGCGGTATC-3′
HP4A:
(SEQ ID NO: 48)
5′-TCATTATCCCAGGCCTAAAA-3′

Gel electrophoresis of the PCR products was used to determine whether fragments of expected size were amplified. Single nested PCR products of the expected size were purified using the QIAquick PCR purification kit (QIAGEN). In addition, expected size products were excised and extracted from agarose gels containing multiple product bands and purified using the QIAquick Gel Extraction kit (QIAGEN).

### Transformation/Isolate Screening/Plasmid Extraction

PCR products were inserted into cloning vectors using the Invitrogen ZERO BLUNT® TOPO® PCR cloning kit, according to the manufacturer's specifications (Life Technologies Corporation, Carlsbad, Calif.). The vector was then transformed into ONE SHOT® TOP10 chemically competent E. coli cells, according to the manufacturer's recommendation and then spread onto LB plates containing 50 ppm of Kanamycin. These plates were incubated in the 37° C. incubator overnight.

To select transformants that contained the vector and DNA insert, colonies were selected from the plate for crude plasmid extraction. 50 mL of DNA Extraction Solution (100 mM NaCl, 10 mM EDTA, 2 mM Tris pH 7) was added to clean 1.5 mL Eppendorf tubes. In the biological hood, 7-10 individual colonies of each TOPO® transformation clone were numbered, picked and resuspended in the extraction solution. In the chemical hood, 50 mL of Phenol:Chloroform:Isoamyl alcohol was added to each sample and vortexed thoroughly. Tubes were microcentrifuged at maximum speed for 5 minutes, after which 20 mL of the top aqueous layer was removed and placed into a clean PCR tubes. 1 mL of RNase (2 mg/mL) was then added, and samples were mixed and incubated at 37° C. tor 30 minutes. The entire sample volume was then run on a gel to determine the presence of the insert in the TOPO® vector based on difference in size to an empty vector. Once the transformant colonies had been identified, those clones was scraped from the plate and used to inoculate separate 15-mL tubes containing 5 mL of LB/Kanamycin medium (0.0001%). The cultures were placed in the 37° C. shaking incubator overnight.

Samples were removed from the incubator and centrifuged for 6 min at 6,000 rpm using the Sorval centrifuge. The QIAprep Spin Miniprep kit (QIAGEN) and protocol were used to isolate the plasmid DNA, which was then digested to confirm the presence of the insert. The restriction enzyme used was dependent on the sites present in and around the insert sequence. Gel electrophoresis was used to determine fragment size. Appropriate DNA samples were submitted for sequencing (Sequetech, Mountain View, Calif.).

### Cloning the 3′ and 5′ Ends

All DNA fragments were sequenced. Sequences were aligned and compared to determine nucleotide and amino acid identities using ALIGNX® and CONTIGEXPRESS® (Vector NTI® suite, Life Sciences Corp., Carlsbad, Calif.). Specific primers were designed to amplify the 3′ and 5′ portions of each incomplete fragment from H. tawa and T. konilangbra by extending outward from the known sequence. At least three specific primers each nested within the amplified product of the previous primer set were designed for each template. Amplification of the 5′ and 3′ sequences was performed using the nested primer sets with the LA PCR In vitro Cloning Kit (Takara Bio Inc., Otsu, Japan)

Fresh genomic DNA was prepared for this amplification. Cultures of T. konilangbra and H. tawa were prepared by inoculating 30 mL of YEG broth with a 1 square inch section of the appropriate sporulated fungal plate culture in 250-mL baffled Erlenmeyer flasks. The flasks were incubated in the 28° C. shaking incubator overnight. The cultures were harvested by centrifugation in 50-mL Sarstedt tubes at 4,500 rpm for 10 minutes. The supernatant was discarded and the mycelia were stored overnight in a −80° C. freezer. The frozen mycelia were then placed into a coffee grinder along with a few pieces of dry ice. The grinder was run until the entire mixture had a powder like consistency. The powder was then air dried and transferred to a sterile 50-mL Sarstedt tube containing 10 mL of EASY-DNA™ Kit Solution A (Life Sciences Corp.) and the manufacturer's protocol was followed. The concentration of the genomic DNA collected from the extraction was measured using the NanoDrop spectrophotometer. The LA PCR In vitro Cloning Kit cassettes were chosen based on the absence of a particular restriction site within the known DNA sequences, and the manufacturer's instructions were followed. For first PCR run, 1 mL of the ligation DNA sample was diluted in 33.5 mL of sterilized distilled water. Different primers were used depending on the sample and the end fragment desired. For the 5′ ends, primers HP4A and TP3A were used for H. tawa and T. konilangbra respectively, while for the 3′ ends primers HP4S and TP3S were used for H. tawa and T. konilangbra. The PCR mixture was prepared by adding 34.5 mL diluted ligation DNA solution, 5 mL of 10× LA Buffer II (Mg²⁺), 8 mL dNTPs mixture, 1 mL cassette primer I, 1 mL specific primer I (depending on sample and end fragment), and 0.5 mL Takara LA Taq polymerase. The PCR tubes were then placed in a thermocycler following the listed protocol:

1. 94° C. for 10 min,

2. 94° C. for 30 s,

3. 55° C. for 30 s,

4. 72° C. for 4 min, return to step 2 30 times,

5. Hold at 4° C.

A second PCR reaction was prepared by taking 1 mL of the first PCR reaction and diluting the sample in sterilized distilled water to a dilution factor of 1:10,000. A second set of primers nested within the first amplified region were used to amplify the fragment isolated in the first PCR reaction. Primers HP3A and TP4A were used to amplify toward the 5′ end of H. tawa and T. konilangbra respectively, while primers HP3S and TP4S were used to amplify toward the 3′ end. The diluted DNA was added to the PCR reaction containing 33.5 mL distilled sterilized water, 5 mL 10× LA Buffer II (Mg²⁺), 8 mL dNTPs mixture, 1 mL of cassette primer 2, 1 mL of specific primer 2 (dependent on sample and fragment, end), 0.5 mL Takara LA Taq, and mixed thoroughly before the PCR run. The PCR protocol was the same as the first reaction, without the initial 94° C. for 10 minutes. After the reaction was complete, the sample was run by gel electrophoresis to determine size and number of fragments isolated. If a single band was present, the sample was purified and sent for sequencing. If no fragment was isolated, a third PCR reaction was performed using the previous protocol for a nested PCR reaction. After running the amplified fragments by gel electrophoresis, the brightest band was excised, purified, and sent for sequencing.

### Analysis of Sequence Alignments

Sequences were obtained and analyzed using the Vector NTI suite, including ALIGNX®, and CONTIGEXPRESS®. Each respective end fragment sequence was aligned to the previously obtained fragments of H. tawa and T. konilangbra to obtain the entire gene sequence. Nucleotide alignments with T. harzianum and T. reesei sequences revealed the translation start and stop points of the gene of interest in both H. tawa and T. konlangbra. After the entire gene sequence was identified, specific primers were designed to amplify the entire gene from the genomic DNA. Primers were designed as described earlier, with the exception of adding CACC nucleotide sequence before the translational starting point, for GATEWAY® cloning (Life Sciences Corp.).

### Primers for Final Cloning:

### T. Konilangbra:

T1FS:
(SEQ ID NO: 49)
caccatgctaggcattctccg
T1FA:
(SEQ ID NO: 50)
tcagcagtattggcatgccg

### H. Tawa:

H1FS:
(SEQ ID NO: 51)
CACCATGTTGGGCGTTTTTCG
H1FA:
(SEQ ID NO: 52)
CTAGCAGTATTGRCATGCCG

The PCR protocol was followed as previously described with the exception of altering the annealing temperature to 55° C. After a single band was isolated and viewed through gel electrophoresis, the amplified fragment was purified as described earlier and used in the pENTR/D TOPO® (Life Sciences Corp.) transformation, according to the manufacturer's instructions. Chemically competent E. coli cells were then transformed as previously described, and transferred to LB plates containing 50 ppm of kanamycin. Following 37° C. incubation overnight, transformants containing the plasmid and insert were selected after crude DNA extraction and plasmid size analysis, as previously described. The selected transformants were scraped from the plate and used to inoculate a fresh 15-mL tube containing 5 mL of LB/Kanamycin medium (0.0001%). Cultures were placed in the 37° C. shaking incubator overnight. Cells were harvested by centrifugation and the plasmid DNA extracted as previously described. Plasm id DNA was digested to confirm the presence of the insert sequence, and then submitted for sequencing. The LR Clonase reaction (Gateway Cloning, Invitrogen (Life Sciences Corp.)) was used, according to manufacturer's instructions, to directionally transfer the insert from the pENTR™/D vector into the destination vector. The destination vector is designed for expression of a gene of interest, in T. reesei, under control of the CBH1 promoter and terminator, with A. niger acetamidase for selection.

### Biolistic Transformation (See General Methods)

### Expression of α-1,3 Glucanases by T. Reesei Transformants

A 1 cm² agar plug was used to inoculate Proflo seed media. Cultures were incubated at 28° C., with 200 rpm Modified amdS Biolistic agar (MABA) per liter shaking. On the second day, a 10% transfer was aseptically made into Production media. The cultures were incubated at 28° C., with 200 rpm shaking. On the third day, cultures were harvested by centrifugation. Supernatants were sterile filtered (0.2 mm polyethersulfone filter; PES) and stored at 4° C. Analysis by SDS-PAGE identified clones expressing the respective alpha-glucanase genes. The growth conditions for the T. reesei transformants followed those used in Example 14.

### Example 18

### Production of Soluble Oligosaccharides using Glucosyltransferase GTF-J (GI:47527) with Simultaneous or Sequential Addition of Mutanase

Reactions (10 mL total volume) were run with 100 g/L sucrose in 50 mM phosphate buffer (pH 6.8) at 35° C., with mixing supplied by a magnetic stir bar. To each reaction was added 0.3% (v/v) concentrated E. coli crude protein extract containing Streptococcus salivarius GTF-J (GI: 47527, GTF7527; Example 3). T. reesei crude protein extract containing either T. konilangbra mutanase or T. reesei 592 mutanase (Example 17) was added at 10% (v/v) of final reaction volume to a reaction either simultaneously with addition of crude protein extract containing GTF-J, or 24 h after addition of crude protein extract containing GTF-J. A control reaction was run with no added mutanase. Aliquots were withdrawn at 4 h and either 22 h or 24 h and quenched by heating at 60° C. for 30 min. Insoluble material was removed from heat-treated samples by centrifugation. The resulting supernatant was analyzed by HPLC to determine the concentration of sucrose, glucose, fructose, leucrose and oligosaccharides (Tables 3 and 4); DP3-DP7 yield was calculated based on sucrose conversion.

TABLE 3
Monosaccharide, disaccharide and oligosaccharide concentrations in reactions containing Streptococcus salivarius
GTF-J and either T. reesei 592 or T. konilangbra mutanase added with GTF-J at start of reaction.

| | mutanase | | | | | | | | | | | | | DP3-DP7 | |
|---|---|---|---|---|---|---|---|---|---|---|---|---|---|---|---|
| Rxn | protein crude | Time | Suc. | Leuc. | Gluc. | Fruc. | DP7 | DP6 | DP5 | DP4 | DP3 | DP2 | DP3-DP7 | yield | Leuc./ |
| # | extract | (h) | (g/L) | (g/L) | (g/L) | (g/L) | (g/L) | (g/L) | (g/L) | (g/L) | (g/L) | (g/L) | (g/L) | (%) | Fruc. |
| 1 | none | 4 | 70.0 | 5.8 | 4.9 | 14.4 | 0.1 | 0.3 | 0.0 | 0.6 | 1.1 | 2.1 | 2.1 | 15 | 0.40 |
| | | 22 | 8.3 | 26.3 | 7.2 | 38.2 | 0.1 | 0.1 | 0.5 | 2.1 | 5.4 | 5.1 | 8.2 | 19 | 0.69 |
| 2 | T. reesei | 4 | 33.8 | 9.7 | 23.1 | 32.9 | 1.1 | 1.1 | 1.6 | 0.6 | 5.0 | 5.3 | 9.4 | 30 | 0.29 |
| | 592 | 22 | 14.0 | 17.8 | 23.7 | 41.7 | 0.3 | 0.3 | 0.3 | 1.7 | 7.6 | 8.6 | 10.2 | 25 | 0.43 |
| | mutanase | | | | | | | | | | | | | | |
| 3 | T. konilangbra | 4 | 61.8 | 8.0 | 5.7 | 17.6 | 0.8 | 1.2 | 1.8 | 2.4 | 1.4 | 2.5 | 7.6 | 42 | 0.45 |
| | mutanase | 22 | 9.6 | 27.1 | 4.9 | 36.1 | 0.3 | 0.3 | 0.8 | 2.4 | 9.5 | 3.7 | 13.3 | 31 | 0.75 |

TABLE 4
Monosaccharide, disaccharide and oligosaccharide concentrations in reactions containing Streptococcus salivarius
GTF-J and either T. reesei 592 or T. konilangbra mutanase added 24 h after GTF-J addition.

| | mutanase | | | | | | | | | | | | | DP3-DP7 | |
|---|---|---|---|---|---|---|---|---|---|---|---|---|---|---|---|
| Rxn | protein crude | Time | Suc. | Leuc. | Gluc. | Fruc. | DP7 | DP6 | DP5 | DP4 | DP3 | DP2 | DP3-DP7 | selectivity | Leuc./ |
| # | extract | (h) | (g/L) | (g/L) | (g/L) | (g/L) | (g/L) | (g/L) | (g/L) | (g/L) | (g/L) | (g/L) | (g/L) | (%) | Fruc. |
| 1 | none | 4 | 8.6 | 26.0 | 7.0 | 38.3 | 0.3 | 0.9 | 0.0 | 1.9 | 2.8 | 4.0 | 5.9 | 14 | 0.68 |
| | | 24 | 9.4 | 26.4 | 6.1 | 38.1 | 0.0 | 0.4 | 0.0 | 1.4 | 2.5 | 5.0 | 4.3 | 10 | 0.69 |
| 2 | T. reesei | 4 | 9.8 | 27.4 | 6.0 | 37.7 | 0.4 | 1.7 | 0.0 | 4.8 | 2.6 | 2.8 | 9.5 | 22 | 0.73 |
| | 592 | 24 | 8.9 | 26.3 | 0.0 | 33.1 | 0.1 | 1.1 | 0.0 | 2.6 | 5.5 | 2.0 | 9.3 | 22 | 0.79 |
| | mutanase | | | | | | | | | | | | | | |
| 3 | T. konilangbra | 4 | 9.8 | 27.6 | 5.7 | 37.4 | 0.4 | 1.5 | 0.0 | 1.5 | 2.5 | 4.9 | 5.9 | 14 | 0.74 |
| | mutanase | 24 | 9.0 | 26.5 | 0.0 | 34.4 | 0.0 | 0.5 | 0.5 | 2.2 | 6.4 | 8.1 | 9.6 | 22 | 0.77 |

### Example 19

### Production of Soluble Oligosaccharides using Glucosyltransferase GTF-J (GI:47527) with Simultaneous or Sequential Addition of Mutanase

Reactions (10 mL total volume) were run with 100 g/L sucrose in 50 mM phosphate buffer (pH 6.8) at 30° C., with mixing supplied by a magnetic stir bar. To each reaction was added 0.3% (v/v) concentrated E. coli crude protein extract containing Streptococcus salivarius GTF-J (GI:47527, GTF7527; Example 3). B. subtilis crude protein extract containing Paenibacillus humicus mutanase (GI:257153264, mut3264; Example 12) was added at 10% (v/v) of final reaction volume to a reaction either simultaneously with addition of crude protein extract containing GTF-J, or 24 h after addition of crude protein extract containing GTF-J. A control reaction was run with no added mutanase. Aliquots were withdrawn at either 4 h or 5 h and either 20 h or 21 h and quenched by heating at 60° C. for 30 min. Insoluble material was removed from heat-treated samples by centrifugation. The resulting supernatant was analyzed by HPLC to determine the concentration of sucrose, glucose, fructose, leucrose and oligosaccharides (Tables 5 and 6); DP3-DP7 yield was calculated based on sucrose conversion.

TABLE 5
Monosaccharide, disaccharide and oligosaccharide concentrations in reactions containing Streptococcus salivarius
GTF-J (GI 47527) and Paenibacillus humicus mutanase (GI: 257153264, mut3264) at start of reaction.

| | Protein | | | | | | | | | | | | | Yield | |
|---|---|---|---|---|---|---|---|---|---|---|---|---|---|---|---|
| Rxn | crude | Time | Suc. | Leuc. | Gluc. | Fruc. | DP7 | DP6 | DP5 | DP4 | DP3 | DP2 | DP3-DP7 | DP3-DP7 | Leuc/ |
| # | extract | (h) | (g/L) | (g/L) | (g/L) | (g/L) | (g/L) | (g/L) | (g/L) | (g/L) | (g/L) | (g/L) | (g/L) | (%) | Fruc |
| 1 | none | 5 | 55.3 | 10.4 | 5.1 | 19.1 | 0.2 | 0.5 | 0.0 | 1.3 | 1.5 | 2.6 | 3.5 | 16.5 | 0.54 |
| | | 21 | 6.0 | 27.6 | 6.6 | 38.5 | 0.5 | 1.2 | 0.0 | 2.3 | 3.2 | 4.3 | 7.2 | 16.2 | 0.72 |
| 2 | Bacillus | 5 | 51.1 | 10.6 | 8.1 | 22.8 | 0.2 | 0.7 | 0.0 | 1.6 | 2.6 | 3.5 | 5.2 | 22.4 | 0.46 |
| | extract | 21 | 7.9 | 27.3 | 6.2 | 40.2 | 0.5 | 1.5 | 0.0 | 3.1 | 3.9 | 4.7 | 8.9 | 20.4 | 0.68 |
| | without | | | | | | | | | | | | | | |
| | mutanase | | | | | | | | | | | | | | |
| 3 | Bacillus | 5 | 40.1 | 12.3 | 7.4 | 28.7 | 0.1 | 1.7 | 0.0 | 5.5 | 3.6 | 3.3 | 11.0 | 38.7 | 0.43 |
| | extract | 21 | 8.7 | 27.0 | 8.5 | 39.8 | 0.1 | 0.2 | 0.6 | 9.9 | 6.8 | 5.9 | 17.7 | 40.9 | 0.68 |
| | with | | | | | | | | | | | | | | |
| | mut3264 | | | | | | | | | | | | | | |

TABLE 6
Monosaccharide, disaccharide and oligosaccharide concentrations in reactions containing
Streptococcus salivarius GTF-J (GI 47527) and Paenibacillus humicus mutanase (GI:
257153264, mut3264), with mutanase added 24 h after start of reaction with GTF-J only.

| | | Time | | | | | | | | | | | | | |
|---|---|---|---|---|---|---|---|---|---|---|---|---|---|---|---|
| | | after | | | | | | | | | | | | | |
| | Protein | mutanase | | | | | | | | | | | | Yield | |
| Rxn | crude | addition | Suc. | Leuc. | Gluc. | Fruc. | DP7 | DP6 | DP5 | DP4 | DP3 | DP2 | DP3-DP7 | DP3-DP7 | Leuc/ |
| # | extract | (h) | (g/L) | (g/L) | (g/L) | (g/L) | (g/L) | (g/L) | (g/L) | (g/L) | (g/L) | (g/L) | (g/L) | (%) | Fruc |
| 1 | none | 4 | 8.6 | 27.7 | 8.8 | 41.0 | 0.5 | 1.3 | 0.0 | 2.5 | 3.4 | 4.6 | 7.7 | 17.8 | 0.68 |
| | | 20 | 9.5 | 30.0 | 5.0 | 40.2 | 0.8 | 1.6 | 0.0 | 2.3 | 3.5 | 4.9 | 8.2 | 19.1 | 0.75 |
| 2 | Bacillus | 4 | 10.3 | 24.6 | 14.2 | 38.1 | 0.1 | 0.2 | 0.3 | 3.4 | 3.7 | 5.3 | 7.7 | 18.1 | 0.65 |
| | extract, | 20 | 12.3 | 29.2 | 9.6 | 37.3 | 0.2 | 0.2 | 0.4 | 3.6 | 6.4 | 6.8 | 10.8 | 26.0 | 0.78 |
| | with | | | | | | | | | | | | | | |
| | mut3264 | | | | | | | | | | | | | | |

### Example 20

### Production of Soluble Oligosaccharides using Combination of Glucosyltransferase GTF-J (GI:47527) Enzyme and Mutanases

Reaction 1 comprised sucrose (100 g/L), E. coli concentrated crude protein extract (0.3% v/v) containing GTF-J from S. salivarius (GI:47527, GTF7527; Example 3) in 50 mM phosphate buffer, pH 6.0. Reactions 2 and 4 comprised sucrose (100 g/L), E. coli concentrated crude protein extract (0.3% v/v) containing GTF-J from S. salivarius (Example 3) and either a T. reesei crude protein extract (10% v/v) comprising a mutanase from Penicillium marneffei ATCC® 18224 (GI:212533325, mut3325; Example 14) or an E. coli crude protein extract (10% v/v) comprising a mutanase from Paenibacillus humicus (GI:257153264, mut3264; Example 12) in 50 mM phosphate buffer, pH 6.0. Control reactions 3 and 5 used either a T. reesei crude protein extract (10% v/v) or an E. coli crude protein extract (10% v/v), respectively, that did not contain mutanase. The total volume for each reaction was 10 mL and all reactions were performed at 40° C. with shaking at 125 rpm. Aliquots were withdrawn at 5 h and 24 h and quenched by heating at 95° C. for 5 min. Insoluble material was removed by centrifugation and filtration. The soluble products were analyzed by HPLC to determine the concentration of sucrose, glucose, fructose, leucrose and oligosaccharides (Table 7). The soluble products from each reaction at 24 h were also analyzed by ¹H NMR spectroscopy to determine the anomeric linkages of the oligosaccharides (Table 8).

TABLE 7
Monosaccharide, disaccharide and oligosaccharide concentrations measured by HPLC.

| | Protein | | | | | | | | | | | | | Yield | |
|---|---|---|---|---|---|---|---|---|---|---|---|---|---|---|---|
| Rxn | crude | Time | Suc. | Leuc. | Gluc. | Fruc. | DP7 | DP6 | DP5 | DP4 | DP3 | DP2 | DP3-DP7 | DP3-DP7 | Leuc/ |
| # | extract | (h) | (g/L) | (g/L) | (g/L) | (g/L) | (g/L) | (g/L) | (g/L) | (g/L) | (g/L) | (g/L) | (g/L) | (%) | Fruc |
| 1 | NA | 5 | 50.5 | 8.7 | 6.9 | 20.6 | 0.0 | 0.0 | 0.3 | 0.7 | 1.2 | 2.4 | 2.2 | 8.9 | 0.42 |
| | | 24 | 0.6 | 25.2 | 8.9 | 38.2 | 0.0 | 0.2 | 0.8 | 1.9 | 2.7 | 3.4 | 5.7 | 11.7 | 0.66 |
| 2 | T. reesei | 5 | 2.9 | 11.6 | 3.2 | 45.1 | 0.1 | 4.3 | 10.2 | 11.6 | 4.8 | 0.6 | 31.0 | 65.6 | 0.26 |
| | extract | 24 | 3.5 | 13.5 | 0.0 | 44.3 | 0.0 | 0.0 | 7.2 | 12.3 | 10.0 | 4.2 | 29.5 | 62.8 | 0.31 |
| | with | | | | | | | | | | | | | | |
| | mut3225 | | | | | | | | | | | | | | |
| 3 | T. reesei | 5 | 58.4 | 10.1 | 7.3 | 18.1 | 0.0 | 0.0 | 0.3 | 1.0 | 1.5 | 2.3 | 2.9 | 14.1 | 0.56 |
| | extract, | 24 | 21.2 | 21.6 | 6.5 | 29.1 | 0.0 | 0.0 | 0.6 | 2.1 | 3.1 | 3.8 | 5.8 | 15.0 | 0.74 |
| | no | | | | | | | | | | | | | | |
| | mutanase | | | | | | | | | | | | | | |
| 4 | E. coli | 5 | 7.5 | 11.6 | 7.2 | 44.0 | 0.0 | 0.0 | 0.6 | 19.3 | 10.3 | 5.4 | 30.2 | 66.7 | 0.26 |
| | extract | 24 | 6.3 | 13.1 | 5.0 | 44.9 | 0.0 | 0.0 | 0.0 | 17.4 | 10.4 | 6.8 | 27.8 | 60.8 | 0.29 |
| | with | | | | | | | | | | | | | | |
| | mut3264 | | | | | | | | | | | | | | |
| 5 | E. coli | 5 | 49.9 | 9.2 | 6.7 | 21.3 | 0.0 | 0.0 | 0.3 | 0.7 | 1.2 | 2.4 | 2.1 | 8.7 | 0.43 |
| | extract, | 24 | 22.0 | 19.5 | 6.2 | 32.0 | 0.0 | 0.0 | 0.6 | 1.3 | 1.9 | 2.8 | 3.8 | 10.0 | 0.61 |
| | no | | | | | | | | | | | | | | |
| | mutanase | | | | | | | | | | | | | | |

TABLE 8
Anomeric linkage analysis of soluble oligosaccharides
by ¹H NMR spectroscopy.

| | Protein | % | % | % | % | % | % |
|---|---|---|---|---|---|---|---|
| Rxn | Crude | α- | α- | α- | α- | α- | α- |
| # | Extract | (1, 4) | (1, 3) | (1, 3, 6) | (1, 2, 6) | (1, 2) | (1, 6) |
| 1 | NA | 14.2 | 47.5 | 5.8 | 0.0 | 0.0 | 32.6 |
| 2 | T. reesei | 2.5 | 93.4 | 0.7 | 0.0 | 0.0 | 3.4 |
| | extract, | | | | | | |
| | mut3325 | | | | | | |
| 3 | T. reesei | 13.8 | 45.8 | 7.8 | 0.0 | 0.0 | 32.5 |
| | extract, | | | | | | |
| | no | | | | | | |
| | mutanase | | | | | | |
| 4 | E. coli | 1.4 | 88.3 | 1.8 | 0.0 | 0.0 | 8.5 |
| | extract, | | | | | | |
| | mut3264 | | | | | | |
| 5 | E. coli | 14.0 | 47.7 | 7.2 | 0.0 | 0.0 | 31.1 |
| | extract, | | | | | | |
| | no | | | | | | |
| | mutanase | | | | | | |

More sucrose was consumed in the first 5 hr of reaction when mutanase was present. Crude extracts from T. reesei and E. coli strains that don't express mutanase didn't have the synergistic effect on sucrose consumption rate. The leucrose to fructose ratios were significantly lower in the presence of mutanases. The yield of soluble oligosaccharides significantly increased in the presence of mutanase. The percentage of α-(1, 3) linkages in the soluble oligosaccharides was substantially increased by the presence of mutanase.

### Example 21

### Production of Soluble Oligosaccharides by GTF-L and Mutanases

Reaction 1 comprised sucrose (100 g/L) and an E. coli protein crude extract (10% v/v) containing GTF-L from Streptococcus salivarius (GI:662379, GTF2379; Example 5) in 50 mM phosphate buffer, pH 6.0. Reactions 2 and 4 comprised sucrose (100 g/L), E. coli protein crude extract (10% v/v) containing GTF-L from Streptococcus salivarius (Example 5) and either a T. reesei crude protein extract (10%, v/v) containing H. tawa mutanase (Example 17) or an E. coli protein crude extract (10%, v/v) containing Paenibacillus humicus (GI:257153264, mut3264; Example 12) in 50 mM phosphate buffer, pH 6.0. Control reactions 3 and 5 used either a T. reesei protein crude extract (10% v/v) or an E. coli protein crude extract (10% v/v), respectively, that did not contain mutanase. The total volume for each reaction was 10 mL and all reactions were performed at 40° C. with shaking at 125 rpm. Aliquots were withdrawn at 5 h and 24 h and reactions were quenched by heating at 95° C. for 5 min. The insoluble materials were removed by centrifugation and filtration. The soluble product mixture was analyzed by HPLC to determine the concentration of sucrose, glucose, fructose, leucrose and oligosaccharides (Table 9). The soluble product from each reaction at 24 h was also analyzed by ¹H NMR spectroscopy to determine the linkages present in the oligosaccharides (Table 10).

TABLE 9
Monosaccharide, disaccharide and oligosaccharide concentrations measured by HPLC.

| | Protein | | | | | | | | | | | | | Yield | |
|---|---|---|---|---|---|---|---|---|---|---|---|---|---|---|---|
| Rxn | crude | Time | Suc. | Leuc. | Gluc. | Fruc. | DP7 | DP6 | DP5 | DP4 | DP3 | DP2 | DP3-DP7 | DP3-DP7 | Leuc/ |
| # | extract | (hr) | (g/L) | (g/L) | (g/L) | (g/L) | (g/L) | (g/L) | (g/L) | (g/L) | (g/L) | (g/L) | (g/L) | (%) | Fruc |
| 1 | NA | 5 | 40.3 | 12.9 | 8.1 | 19.9 | 0.3 | 0.5 | 0.8 | 1.2 | 1.5 | 3.6 | 4.3 | 14.9 | 0.65 |
| | | 24 | 5.2 | 27.8 | 8.6 | 34.5 | 1.8 | 2.4 | 3.0 | 3.3 | 3.7 | 6.7 | 14.1 | 30.6 | 0.81 |
| 2 | T. reesei | 5 | 28.4 | 17.8 | 25.8 | 44.2 | 0.2 | 0.7 | 1.4 | 2.4 | 6.2 | 8.0 | 11.0 | 31.3 | 0.40 |
| | extract, | 24 | 8.4 | 19.4 | 20.8 | 40.6 | 0.3 | 0.8 | 1.6 | 2.3 | 4.4 | 9.7 | 9.3 | 20.8 | 0.48 |
| | H. tawa | | | | | | | | | | | | | | |
| | mutanase | | | | | | | | | | | | | | |
| 3 | T. reesei | 5 | 41.9 | 13.3 | 8.5 | 20.7 | 0.3 | 0.6 | 0.9 | 1.3 | 1.6 | 3.8 | 4.6 | 16.2 | 0.64 |
| | extract, | 24 | 5.1 | 28.4 | 8.1 | 34.5 | 1.8 | 2.5 | 2.9 | 3.3 | 3.8 | 7.2 | 14.3 | 30.9 | 0.82 |
| | no | | | | | | | | | | | | | | |
| | mutanase | | | | | | | | | | | | | | |
| 4 | E. coli | 5 | 28.4 | 16.7 | 10.6 | 42.6 | 0.7 | 1.2 | 2.4 | 13.2 | 6.9 | 9.0 | 24.3 | 69.6 | 0.39 |
| | extract, | 24 | 3.3 | 19.0 | 8.7 | 40.4 | 0.3 | 1.0 | 2.0 | 6.9 | 6.9 | 13.2 | 17.1 | 36.3 | 0.47 |
| | mut3264 | | | | | | | | | | | | | | |
| 5 | E. coli | 5 | 48.1 | 17.1 | 10.4 | 26.2 | 0.00 | 3.5 | 3.5 | 5.8 | 4.7 | 6.3 | 17.5 | 69.2 | 0.65 |
| | extract, | 24 | 5.1 | 28.2 | 8.7 | 34.4 | 1.9 | 2.6 | 3.2 | 3.5 | 3.9 | 6.9 | 15.0 | 32.6 | 0.82 |
| | no | | | | | | | | | | | | | | |
| | mutanase | | | | | | | | | | | | | | |

TABLE 10
Anomeric linkage analysis of soluble oligosaccharides
by ¹H NMR spectroscopy.

| | Protein | % | % | % | % | % | % |
|---|---|---|---|---|---|---|---|
| Rxn | Crude | α- | α- | α- | α- | α- | α- |
| # | Extract | (1, 4) | (1, 3) | (1, 3, 6) | (1, 2, 6) | (1, 2) | (1, 6) |
| 1 | NA | 9.7 | 14.3 | 7.2 | 0.0 | 0.0 | 68.8 |
| 2 | T. reesei | 12.3 | 23.2 | 5.3 | 0.0 | 0.0 | 59.3 |
| | extract, | | | | | | |
| | H. tawa | | | | | | |
| | mutanase | | | | | | |
| 3 | T. reesei | 10.2 | 13.3 | 7.4 | 0.0 | 0.0 | 69.1 |
| | extract, | | | | | | |
| | no | | | | | | |
| | mutanase | | | | | | |
| 4 | E. coli | 6.3 | 56.4 | 3.1 | 0.0 | 0.0 | 34.3 |
| | extract, | | | | | | |
| | mut3264 | | | | | | |
| 5 | E. coli | 10.0 | 13.8 | 7.5 | 0.0 | 0.0 | 68.8 |
| | extract, | | | | | | |
| | no | | | | | | |
| | mutanase | | | | | | |

More sucrose was consumed in the first 5 h when mutanase was present. Crude extracts from T. reesei and E. coli strains that don't express mutanase don't have the synergistic effect on sucrose consumption rate. Less leucrose was produced in the presence of mutanase after 24 h when sucrose consumption was near completion. The leucrose to fructose ratios were significantly lower in the presence of mutanases. The amount of soluble oligosaccharides of DP3 to DP7 significantly increased in the presence of mut3264. More glucose was produced in the reaction with H. tawa mutanase than in other reactions. The percentage of α-(1,3) linkages in the soluble oligosaccharides was substantially increased by the presence of mutanase.

### Example 22

### Production of Soluble Oligosaccharides by GTF-B and Mutanases

Reaction 1 comprised sucrose (100 g/L) and E. coli protein crude extract (10% v/v) containing GTF-B from Streptococcus mutans NN2025 (GI:290580544, GTF0544; Example 6) in 50 mM phosphate buffer, pH 6.0. Reactions 2 and 4 below comprised sucrose (100 g/L), E. coli protein crude extract (10% v/v) containing GTF-B from Streptococcus mutans NN2025 (GI:290580544, GTF0544; Example 6) and either a T. reesei protein crude extract (10%, v/v) containing H. tawa mutanase (Example 17) or an E. coli protein crude extract (10%, v/v) containing Paenibacillus humicus mutanase(GI:257153264, mut3264; Example 12) in 50 mM phosphate buffer, pH 6.0. Control reactions 3 and 5 used either a T. reesei crude protein extract (10% v/v) or an E. coli crude protein extract (10% v/v), respectively, that did not contain mutanase. The total volume for each reaction was 10 mL and all reactions were performed at 40° C. with shaking at 125 rpm. Aliquots were withdrawn at 5 h and 24 h and reactions were quenched by heating aliquot samples at 95° C. for 5 min. The insoluble materials were removed by centrifugation and filtration, and the resulting filtrate was analyzed by HPLC to determine the concentration of sucrose, glucose, fructose, leucrose and oligosaccharides (Table 11). The soluble product from each reaction at 24 h was also analyzed by ¹H NMR spectroscopy to determine the linkage of the oligosaccharides (Table 12).

TABLE 11
Monosaccharide, disaccharide and oligosaccharide concentrations measured by HPLC.

| | Protein | | | | | | | | | | | | | Yield | |
|---|---|---|---|---|---|---|---|---|---|---|---|---|---|---|---|
| Rxn | crude | Time | Suc. | Leuc. | Gluc. | Fruc. | DP7 | DP6 | DP5 | DP4 | DP3 | DP2 | DP3-DP7 | DP3-DP7 | Leuc/ |
| # | extract | (hr) | (g/L) | (g/L) | (g/L) | (g/L) | (g/L) | (g/L) | (g/L) | (g/L) | (g/L) | (g/L) | (g/L) | (%) | Fruc |
| 1 | NA | 5 | 77.1 | 3.1 | 2.9 | 14.2 | 0.0 | 0.3 | 0.5 | 0.5 | 0.3 | 0.6 | 1.5 | 13.9 | 0.22 |
| | | 24 | 28.7 | 14.3 | 2.0 | 31.1 | 1.9 | 2.5 | 2.6 | 1.9 | 1.0 | 1.7 | 9.8 | 28.4 | 0.46 |
| 2 | T. reesei | 5 | 69.5 | 3.3 | 10.4 | 22.0 | 0.0 | 0.3 | 0.8 | 0.8 | 2.0 | 1.8 | 3.9 | 26.3 | 0.15 |
| | extract, | 24 | 11.6 | 11.5 | 13.1 | 40.4 | 1.1 | 2.3 | 3.0 | 2.2 | 2.4 | 4.3 | 10.9 | 25.5 | 0.29 |
| | H. tawa | | | | | | | | | | | | | | |
| | mutanase | | | | | | | | | | | | | | |
| 3 | T. reesei | 5 | 74.6 | 3.1 | 3.0 | 14.1 | 0.0 | 0.3 | 0.5 | 0.5 | 0.3 | 0.7 | 1.6 | 12.8 | 0.22 |
| | extract, | 24 | 30.4 | 14.6 | 3.1 | 29.8 | 2.0 | 2.7 | 2.8 | 2.4 | 1.9 | 2.3 | 11.8 | 35.0 | 0.49 |
| | no | | | | | | | | | | | | | | |
| | mutanase | | | | | | | | | | | | | | |
| 4 | E. coli | 5 | 59.4 | 3.2 | 3.0 | 21.8 | 0.2 | 1.0 | 2.0 | 5.2 | 2.5 | 2.6 | 10.8 | 54.6 | 0.15 |
| | extract, | 24 | 5.7 | 11.2 | 1.5 | 43.6 | 2.4 | 5.1 | 5.9 | 6.0 | 4.3 | 5.2 | 23.7 | 51.8 | 0.26 |
| | mut3264 | | | | | | | | | | | | | | |
| 5 | E. coli | 5 | 32.3 | 10.9 | 3.5 | 29.8 | 1.1 | 1.5 | 1.4 | 0.9 | 0.5 | 1.0 | 5.4 | 16.5 | 0.36 |
| | extract, | 24 | 0.2 | 19.9 | 1.7 | 38.2 | 2.6 | 2.9 | 2.5 | 1.6 | 0.6 | 1.9 | 10.3 | 21.3 | 0.52 |
| | no | | | | | | | | | | | | | | |
| | mutanase | | | | | | | | | | | | | | |

TABLE 12
Linkage analysis of soluble oligosaccharides in each reaction by
¹H NMR spectroscopy.

| | Protein | % | % | % | % | | |
|---|---|---|---|---|---|---|---|
| | Crude | α- | α- | α- | α- | % | % |
| Rxn # | Extract | (1,4) | (1,3) | (1,3,6) | (1,2,6) | α-(1,2) | α-(1,6) |
| 1 | NA | 6.3 | 15.4 | 3.0 | 0.0 | 0.0 | 75.3 |
| 2 | T. reesei | 3.5 | 15.9 | 5.6 | 0.0 | 0.0 | 75.1 |
| | extract, | | | | | | |
| | H. tawa | | | | | | |
| | mutanase | | | | | | |
| 3 | T. reesei | 6.4 | 17.8 | 3.3 | 0.0 | 0.0 | 72.5 |
| | extract, | | | | | | |
| | no | | | | | | |
| | mutanase | | | | | | |
| 4 | E. coli | 2.1 | 31.9 | 3.4 | 0.0 | 0.0 | 62.7 |
| | extract, | | | | | | |
| | mut3264 | | | | | | |
| 5 | E. coli | 4.8 | 9.4 | 2.7 | 0.0 | 0.0 | 83.1 |
| | extract, | | | | | | |
| | no | | | | | | |
| | mutanase | | | | | | |

More sucrose was consumed in the first 5 hr when mutanase was present. Crude protein extracts from T. reesei that did not express mutanase did not have the synergistic effect on sucrose consumption rate. More oligosaccharides of DP3-DP7 were produced in the presence of mut3264, but not in the presence of H. tawa mutanase or the two protein extracts without mutanase. Less leucrose was produced in the presence of mutanase after 24 h when sucrose consumption was near completion. The leucrose to fructose ratios were significantly lower in the presence of mutanases. High concentration of glucose was produced in the presence of the H. tawa mutanase.

The percentage of α-(1,3) linkages in the soluble oligosaccharides was substantially increased by the presence of mut3264.

### Example 23

### Production of Soluble Oligosaccharides by GTF-I and MUT3264 Mutanase

Reaction 1 comprised sucrose (100 g/L) and E. coli protein crude extract (3% v/v) containing the GTF-I from Streptococcus sobrinus (GI:450874, GTF0874; Example 8) in 50 mM phosphate buffer (pH 6.0). Reaction 2 comprised sucrose (100 g/L), E. coli protein crude extract (3% v/v) containing GTF-I from Streptococcus sobrinus (Example 8) and an B. subtilis protein crude extract (10%, v/v) containing Paenibacillus humicus mutanase (mut3264, GI:257153264, Example 13) in 50 mM phosphate buffer (pH 6.8). The total volume for each reaction was 10 mL and all reactions were performed at 30° C. with stirring by magnetic stir bar. Aliquots were withdrawn at 5 h, 24 h and 48 h, and reactions were quenched by heating aliquoted samples at 60° C. for 30 min. The insoluble materials were removed by centrifugation, and the resulting supernatant was analyzed by HPLC to determine the concentration of sucrose, glucose, fructose, leucrose and oligosaccharides (Table 13).

TABLE 13
Monosaccharide, disaccharide and oligosaccharide concentrations measured by HPLC.

| | mutanase | | | | | | | | | | | | | | |
|---|---|---|---|---|---|---|---|---|---|---|---|---|---|---|---|
| | protein | | | | | | | | | | | | | Yield | |
| | crude | Time | Suc. | Leuc. | Gluc. | Fruc. | DP7 | DP6 | DP5 | DP4 | DP3 | DP2 | DP3-DP7 | DP3-DP7 | Leuc/ |
| Rxn # | extract | (hr) | (g/L) | (g/L) | (g/L) | (g/L) | (g/L) | (g/L) | (g/L) | (g/L) | (g/L) | (g/L) | (g/L) | (%) | Fruc |
| 1 | none | 5 | 1.6 | 40.8 | 8.9 | 27.5 | 1.5 | 2.5 | 0.0 | 3.0 | 2.6 | 1.6 | 9.6 | 20.6 | 1.48 |
| | | 24 | 1.6 | 37.5 | 11.0 | 33.3 | 1.2 | 0.0 | 2.2 | 2.9 | 3.5 | 4.8 | 9.8 | 21.0 | 1.13 |
| | | 48 | 3.2 | 31.7 | 7.3 | 32.9 | 2.3 | 0.0 | 2.4 | 3.2 | 3.9 | 5.8 | 11.8 | 25.7 | 0.96 |
| 2 | Bacillus | 5 | 3.6 | 33.0 | 9.8 | 31.5 | 0.3 | 2.5 | 0.0 | 6.4 | 5.7 | 5.1 | 14.9 | 32.6 | 1.05 |
| | extract | 24 | 6.7 | 32.1 | 11.0 | 33.3 | 0.3 | 0.6 | 1.7 | 4.5 | 5.9 | 8.8 | 13.0 | 29.4 | 0.96 |
| | containing | 48 | 6.5 | 28.2 | 11.8 | 32.1 | 0.5 | 1.2 | 2.7 | 5.6 | 6.2 | 9.2 | 16.2 | 36.6 | 0.88 |
| | mut3264 | | | | | | | | | | | | | | |

### Example 24

### The Effect of GTF-I Glucosyltransferase and MUT3325 Mutanase Ratios on Oligosaccharides Production

Reactions 1-4 comprised sucrose (100 g/L), a T. reesei protein crude extract (10% v/v) containing Penicillium marneffei ATCC® 18224 mutanase (mut3325); Example 14), and an E. coli protein crude extract containing GTF-I from Streptococcus sobrinus (GI:450874, GTF0874; Example 8) at one of 0.5%, 2.5%, 5% or 10% (v/v) in 50 mM potassium phosphate buffer at pH 5.4. Reactions 6-9 comprised sucrose (100 g/L), no added MUT3325, and an E. coli protein crude extract containing GTF-I from Streptococcus sobrinus (GI:450874; Example 4) at one of 0.5%, 2.5% , 5% or 10% (v/v) in 50 mM potassium phosphate buffer at pH 5.4. Reaction 5 contained only sucrose (100 g/L) in the same buffer. All reactions were performed at 37° C. with shaking at 125 rpm. Aliquots (500 μL) were withdrawn from each reaction at 1 h, 5 h and 25 h, and heated at 90° C. for 5 min to stop the reaction. Insoluble materials were removed by centrifugation and filtration. The resulting filtrate was analyzed by HPLC to determine the concentration of sucrose (Suc.), glucose (Gluc.), fructose (Fruc.), leucrose (Leuc.) and oligosaccharides (DP3-7) (Tables 14-16).

TABLE 14
Monosaccharide, disaccharide and oligosaccharide concentrations measured by HPLC (1 h).

| | | | | | | | | | | | | | | Yield |
|---|---|---|---|---|---|---|---|---|---|---|---|---|---|---|
| | GTF-I | mut3325 | Suc. | Leuc. | Gluc. | Fruc. | DP7 | DP6 | DP5 | DP4 | DP3 | DP2 | DP3-DP7 | DP3-DP7 |
| Rxn # | % (v/v) | % (v/v) | (g/L) | (g/L) | (g/L) | (g/L) | (g/L) | (g/L) | (g/L) | (g/L) | (g/L) | (g/L) | (g/L) | (%) |
| 1 | 10 | 10 | 42.3 | 11.7 | 3.2 | 25.0 | 0.0 | 6.7 | 1.8 | 5.3 | 0.0 | 0.0 | 13.9 | 49.5 |
| 2 | 5 | 10 | 69.8 | 5.0 | 2.6 | 13.7 | 0.2 | 1.2 | 2.1 | 2.3 | 1.0 | 0.0 | 6.9 | 47.2 |
| 3 | 2.5 | 10 | 84.5 | 1.5 | 1.9 | 7.6 | 0.0 | 0.6 | 1.3 | 1.7 | 0.8 | 0.0 | 4.3 | 57.0 |
| 4 | 0.5 | 10 | 90.4 | 0.0 | 1.0 | 5.1 | 0.0 | 0.4 | 0.9 | 1.4 | 0.7 | 0.0 | 3.3 | 71.6 |
| 5 | 0 | 0 | 99.5 | 0.0 | 0.9 | 0.0 | 0.0 | 0.0 | 0.0 | 0.0 | 0.0 | 0.0 | 0.0 | 0.0 |
| 6 | 10 | 0 | 63.1 | 9.1 | 4.9 | 14.3 | 0.0 | 0.4 | 1.0 | 1.1 | 0.9 | 0.6 | 3.3 | 18.5 |
| 7 | 5 | 0 | 85.4 | 2.6 | 3.7 | 6.3 | 0.0 | 0.0 | 0.2 | 0.4 | 0.4 | 0.3 | 1.1 | 15.2 |
| 8 | 2.5 | 0 | 92.4 | 0.7 | 2.6 | 3.0 | 0.0 | 0.0 | 0.0 | 0.0 | 0.0 | 0.0 | 0.0 | 0.0 |
| 9 | 0.5 | 0 | 97.9 | 0.0 | 1.1 | 0.7 | 0.0 | 0.0 | 0.0 | 0.0 | 0.0 | 0.0 | 0.0 | 0.0 |

TABLE 15
Monosaccharide, disaccharide and oligosaccharide concentrations measured by HPLC (5 h).

| | | | | | | | | | | | | | | Yield |
|---|---|---|---|---|---|---|---|---|---|---|---|---|---|---|
| | GTF-I | mut3325 | Suc. | Leuc. | Gluc. | Fruc. | DP7 | DP6 | DP5 | DP4 | DP3 | DP2 | DP3-DP7 | DP3-DP7 |
| Rxn # | % (v/v) | % (v/v) | (g/L) | (g/L) | (g/L) | (g/L) | (g/L) | (g/L) | (g/L) | (g/L) | (g/L) | (g/L) | (g/L) | (%) |
| 1 | 10 | 10 | 0.7 | 27.7 | 3.9 | 38.3 | 0.0 | 2.0 | 4.5 | 5.2 | 3.3 | 0.7 | 14.9 | 30.8 |
| 2 | 5 | 10 | 14.1 | 26.1 | 4.3 | 31.8 | 0.7 | 3.4 | 6.3 | 6.3 | 2.6 | 0.4 | 19.3 | 46.3 |
| 3 | 2.5 | 10 | 59.6 | 9.5 | 3.5 | 16.8 | 0.0 | 1.0 | 3.0 | 3.5 | 1.8 | 0.6 | 9.3 | 47.2 |
| 4 | 0.5 | 10 | 78.1 | 1.3 | 1.7 | 11.2 | 0.0 | 0.6 | 2.3 | 3.3 | 1.8 | 0.2 | 8.0 | 75.3 |
| 5 | 0 | 0 | 99.5 | 0.0 | 1.2 | 0.0 | 0.0 | 0.0 | 0.0 | 0.0 | 0.0 | 0.0 | 0.0 | 0.0 |
| 6 | 10 | 0 | 0.4 | 34.3 | 6.4 | 33.5 | 0.8 | 1.9 | 2.6 | 2.3 | 1.2 | 1.4 | 8.8 | 18.1 |
| 7 | 5 | 0 | 42.6 | 17.9 | 5.8 | 21.6 | 0.2 | 0.9 | 1.7 | 1.6 | 1.1 | 0.6 | 5.5 | 19.5 |
| 8 | 2.5 | 0 | 73.8 | 6.5 | 4.6 | 10.8 | 0.0 | 0.2 | 0.7 | 0.9 | 0.7 | 0.5 | 2.5 | 19.3 |
| 9 | 0.5 | 0 | 94.9 | 0.4 | 2.2 | 2.2 | 0.0 | 0.0 | 0.0 | 0.0 | 0.0 | 0.0 | 0.0 | 0.0 |

TABLE 16
Monosaccharide, disaccharide and oligosaccharide concentrations measured by HPLC (25 h).

| | | | | | | | | | | | | | | Yield |
|---|---|---|---|---|---|---|---|---|---|---|---|---|---|---|
| | GTF-I | mut3325 | Suc. | Leuc. | Gluc. | Fruc. | DP7 | DP6 | DP5 | DP4 | DP3 | DP2 | DP3-DP7 | DP3-DP7 |
| Rxn # | % (v/v) | % (v/v) | (g/L) | (g/L) | (g/L) | (g/L) | (g/L) | (g/L) | (g/L) | (g/L) | (g/L) | (g/L) | (g/L) | (%) |
| 1 | 10 | 10 | 4.8 | 29.4 | 2.8 | 34.8 | 0.0 | 0.7 | 1.9 | 4.0 | 6.1 | 6.9 | 12.7 | 27.4 |
| 2 | 5 | 10 | 4.0 | 33.4 | 3.2 | 33.0 | 0.0 | 0.5 | 3.7 | 6.4 | 7.5 | 5.8 | 18.1 | 38.6 |
| 3 | 2.5 | 10 | 2.7 | 33.7 | 4.2 | 33.9 | 0.0 | 1.4 | 5.9 | 8.0 | 6.9 | 4.5 | 22.2 | 46.7 |
| 4 | 0.5 | 10 | 34.4 | 14.6 | 3.6 | 27.1 | 0.0 | 0.8 | 6.0 | 7.8 | 4.9 | 2.5 | 19.4 | 60.8 |
| 5 | 0 | 0 | 98.0 | 0.0 | 1.5 | 0.9 | 0.0 | 0.0 | 0.0 | 0.0 | 0.0 | 0.0 | 0.0 | 0.0 |
| 6 | 10 | 0 | 0.5 | 33.6 | 5.8 | 34.2 | 0.7 | 1.7 | 2.3 | 2.2 | 1.8 | 0.9 | 8.7 | 17.9 |
| 7 | 5 | 0 | 0.4 | 34.8 | 5.7 | 33.1 | 0.8 | 2.0 | 2.6 | 2.3 | 1.5 | 1.6 | 9.2 | 19.0 |
| 8 | 2.5 | 0 | 0.5 | 36.9 | 6.0 | 32.8 | 0.9 | 2.2 | 3.1 | 2.8 | 1.3 | 0.0 | 10.3 | 21.3 |
| 9 | 0.5 | 0 | 74.1 | 7.3 | 4.7 | 10.8 | 0.2 | 0.7 | 1.0 | 0.8 | 0.5 | 0.0 | 3.1 | 24.9 |

A comparison of the data in Tables 14, 15, and 16 shows that sucrose conversion was faster in the presence of mut3325 at all concentrations of GTF-I. The total amount and yield of DP3 to DP7 significantly increased in the reactions in the presence of mut3325. Higher mut3325 to GTF-I ratio resulted in higher yields of DP3-DP7 oligosaccharides.

### Example 25

### The Effect of the GTF-J Glucosyltransferase and MUT3325 Mutanase Ratios on Oligosaccharides Production

The reactions 1-3 below comprised 200 g/L sucrose, varied concentrations of GTF-J (GTF-J from S. salivarius; GI:47527, Example 3) (0.6 and 1% v/v) and varied concentrations of mut3325 (Penicillium marneffei ATCC® 18224 mutanase; Example 14) (10 and 20%) as indicated in the Table 17. All reactions were performed at 37° C. with tilt shaking at 125 rpm. The reactions were quenched after 16-19 h by heating at 90° C. for 5 min. The insoluble materials were removed by centrifugation and filtration. The soluble product mixture was analyzed by HPLC to determine the concentration of sucrose, glucose, fructose, leucrose and oligosaccharides (Table 17). The data in Table 17 shows that a higher ratio of mut3325 to GTF-J produced a higher yield of soluble DP3 to DP7oligosaccharides.

TABLE 17
Monosaccharide, disaccharide and oligosaccharide concentrations measured by HPLC (25 h).

| | | | | | | | | | | | | | | Yield |
|---|---|---|---|---|---|---|---|---|---|---|---|---|---|---|
| | GTF-J | mut3325 | Suc. | Leuc. | Gluc. | Fruc. | DP7 | DP6 | DP5 | DP4 | DP3 | DP2 | DP3-DP7 | DP3-DP7 |
| Rxn # | % (v/v) | % (v/v) | (g/L) | (g/L) | (g/L) | (g/L) | (g/L) | (g/L) | (g/L) | (g/L) | (g/L) | (g/L) | (g/L) | (%) |
| 1 | 1 | 10 | 1.6 | 56.0 | 2.9 | 70.0 | 0 | 0 | 4.0 | 6.1 | 6.8 | 2.6 | 16.9 | 17.5 |
| 2 | 0.6 | 10 | 1.0 | 54.4 | 3.2 | 71.0 | 0 | 0.2 | 7.6 | 8.7 | 8.7 | 2.2 | 25.3 | 26.0 |
| 3 | 0.6 | 20 | 5.1 | 50.0 | 0.0 | 78.2 | 0 | 0.2 | 12.6 | 17.4 | 15.0 | 8.9 | 45.2 | 47.6 |

### Example 26

### Effect of pH on the Oligosaccharide Production

The reactions 1-3 below comprised of sucrose (100 g/L), gtf-J (0.3% by volume, Example 3) and E. coli crude protein extract containing mut3264 mutanase (10% volume, Example 12) at pH 5.0, 6.0 and 6.8. The buffers used for various pH were: 50 mM citrate buffer, pH 5.0; 50 mM phosphate, pH. 6.0 and 50 mM phosphate pH 6.8. The reactions were carried out at 30° C. with shaking at 125 rpm. Aliquots from each reaction were withdrawn at 5 hr, 24 hr, 48 hr and 72 hr and quenched by heating at 90° C. for 5 min. The insoluble materials were removed by centrifugation and filtration. The soluble product mixture was analyzed by HPLC to determine the concentration of sucrose, glucose, fructose, leucrose and oligosaccharides (Table 18). The data in Table 18 shows that DP4 oligosaccharide produced at pH 5.0 and pH 6.8 was further degraded by the mutanase to smaller DPs with prolonged incubation, while no further degradation was observed at pH 6.0.

TABLE 18
Monosaccharide, disaccharide and oligosaccharide concentrations measured by HPLC.

| | GTF- | E. coli | | | | | | | | | | | | | |
|---|---|---|---|---|---|---|---|---|---|---|---|---|---|---|---|
| | J % | mut3264 | | Time | Suc. | Leuc. | Gluc. | Fruc. | DP7 | DP6 | DP5 | DP4 | DP3 | DP2 | DP3-DP7 |
| Rxn# | (v/v) | % (v/v) | pH | (h) | (g/L) | (g/L) | (g/L) | (g/L) | (g/L) | (g/L) | (g/L) | (g/L) | (g/L) | (g/L) | (g/L) |
| 1 | 0.3 | 10 | 5.0 | 5 | 46.4 | 12.8 | 3.5 | 30.8 | 0.0 | 0.1 | 0.0 | 13.7 | 7.7 | 4.0 | 21.6 |
| | | | | 24 | 12.2 | 19.1 | 2.2 | 43.9 | 0.0 | 0.0 | 0.0 | 14.7 | 11.9 | 8.7 | 26.6 |
| | | | | 48 | 18.3 | 19.1 | 0.9 | 43.3 | 0.0 | 0.0 | 0.0 | 9.1 | 14.2 | 15.1 | 23.3 |
| | | | | 72 | 25.6 | 22.0 | 2.3 | 43.5 | 0.0 | 0.0 | 0.0 | 4.4 | 13.3 | 18.2 | 17.7 |
| 2 | 0.3 | 10 | 6.0 | 5 | 38.3 | 10.2 | 3.9 | 30.8 | 0.0 | 0.1 | 0.0 | 13.8 | 8.1 | 4.1 | 22.0 |
| | | | | 24 | 9.6 | 19.1 | 4.3 | 41.0 | 0.0 | 0.0 | 0.0 | 14.8 | 11.0 | 8.1 | 25.8 |
| | | | | 48 | 10.7 | 20.5 | 4.7 | 43.5 | 0.0 | 0.0 | 0.0 | 15.0 | 11.5 | 8.5 | 26.5 |
| | | | | 72 | 9.3 | 18.2 | 2.1 | 40.4 | 0.0 | 0.0 | 0.0 | 14.4 | 11.2 | 8.2 | 25.6 |
| 3 | 0.3 | 10 | 6.8 | 5 | 39.2 | 9.4 | 3.6 | 29.0 | 0.0 | 0.1 | 0.0 | 13.4 | 7.2 | 3.7 | 20.8 |
| | | | | 24 | 8.7 | 18.9 | 1.7 | 40.1 | 0.0 | 0.0 | 0.0 | 13.8 | 11.5 | 8.9 | 25.3 |
| | | | | 48 | 13.7 | 19.1 | 0.9 | 40.1 | 0.0 | 0.0 | 0.0 | 8.9 | 12.5 | 13.6 | 21.4 |
| | | | | 72 | 14.3 | 18.6 | 0.1 | 39.0 | 0.0 | 0.0 | 0.0 | 7.7 | 12.7 | 14.3 | 20.4 |

### Example 27

### Effect of Temperature on the Oligosaccharide Production

The reactions 1-4 below comprised of sucrose (100 g/L), phosphate buffer (50 mM, pH 6.0), GTF-J (0.3% by volume, Example 3) and E. coli crude extract of mut3264 mutanase (10% by volume, Example 12). The reactions were carried out at 30° C., 40° C., 50° C. and 60° C. as specified in Table 19 with shaking at 125 rpm. The reactions were quenched after 24 hr by heating at 90° C. for 5 min. The insoluble materials were removed by centrifugation and filtration. The soluble product mixture was analyzed by HPLC to determine the concentration of sucrose, glucose, fructose, leucrose and oligosaccharides (Table 19). The total amount of oligosaccharides of DP3 to DP7 was the highest at 40° C.

TABLE 19
Monosaccharide, disaccharide and oligosaccharide concentrations measured by HPLC.

| | GTF- | E. coli | | | | | | | | | | | | | |
|---|---|---|---|---|---|---|---|---|---|---|---|---|---|---|---|
| | J % | mut3264 | Temp. | Time | Suc. | Leuc. | Gluc. | Fruc. | DP7 | DP6 | DP5 | DP4 | DP3 | DP2 | DP3-DP7 |
| Rxn# | (v/v) | % (v/v) | (° C.) | (h) | (g/L) | (g/L) | (g/L) | (g/L) | (g/L) | (g/L) | (g/L) | (g/L) | (g/L) | (g/L) | (g/L) |
| 1 | 0.3 | 10 | 30 | 24 | 11.0 | 17.3 | 3.9 | 41.2 | 0 | 0.00 | 0 | 15.0 | 11.0 | 7.7 | 26.0 |
| 2 | 0.3 | 10 | 40 | 24 | 7.1 | 12.5 | 5.7 | 46.2 | 0 | 0.00 | 0 | 20.5 | 12.3 | 7.6 | 32.8 |
| 3 | 0.3 | 10 | 50 | 24 | 60.8 | 8.9 | 7.6 | 20.9 | 0 | 0.00 | 0 | 2.4 | 4.5 | 5.3 | 6.9 |
| 4 | 0.3 | 10 | 60 | 24 | 103.5 | 0.0 | 0.4 | 1.2 | 0 | 0.00 | 0 | 0.2 | 0.0 | 0.0 | 0.2 |

### Example 28

### Effect of MUT6505 Mutanase on the Sucrose Consumption by GTF-J

Various concentrations of a T. reesei crude protein extract containing mut6505 (Aspergillus nidulans FGSC A4 mutanase GI:259486505; Example 16) as indicated in Table 20 (below) were incubated with 100 g/L sucrose, and 0.3% (v/v) of an E. coli crude protein extract containing GTF-J (Example 3) in final volumes of 1 mL. The reactions were incubated at 37° C. with shaking 150 rpm for 3 h. Reactions were quenched by heating at 90° C. for 3 min. The insoluble materials were removed by centrifugation and filtration through 0.2 μm sterile filter. The filtrate was analyzed on HPLC as described in the general methods. The data (Table 20) show that faster sucrose consumption correlates with increased mutanase concentration.

TABLE 20
Effect of mut6505 mutanase on sucrose conversion by GTF-J.
100 g/L sucrose, 0.3% (v/v) GTF-J extract, 37° C., 3 h

| | 10% | 4% | 1% |
|---|---|---|---|
| | mut6505 | mut6505 | mut6505 |
| DP6 | 0.0 | 0.0 | 0.0 |
| DP5 | 0.0 | 0.0 | 0.0 |
| DP4 | 0.3 | 0.2 | 0.0 |
| DP3 | 2.8 | 1.4 | 0.8 |
| DP2 | 3.1 | 2.0 | 1.6 |
| Sucrose | 48.9 | 71.5 | 78.9 |
| Leucrose | 8.7 | 4.8 | 3.1 |
| Glucose | 16.2 | 8.4 | 6.2 |
| Fructose | 23.5 | 12.8 | 9.7 |
| DP2-DP7 | 6.1 | 3.6 | 2.4 |
| DP3-DP7 | 3.0 | 1.6 | 0.8 |
| Total | 103.3 | 101.2 | 100.4 |

### Example 29

### Digestibility of the Oligosaccharides Produced by the Combination of a GTF and Mutanase

The DP3-DP7 oligosaccharides from the glucosyltransferase and mutanase reactions were purified on the SEC column as described in the general methods.

The digestibility test protocol was adapted from the Megazyme Integrated Total Dietary Fiber Assay (AOAC method 2009.01, Ireland). The final enzyme concentrations were kept the same as the AOAC method: 50 Unit/mL of pancreatic α-amylase (PAA), 3.4 Units/mL for amyloglucosidase (AMG). The substrate concentration in each reaction was 25 mg/mL as recommended by the AOAC method. The total volume for each reaction was 1 mL. Every sample was analyzed in duplicate with and without the treatment of the two digestive enzymes. The amount of released glucose was quantified by HPLC with the Am inex HPX-87C Columns (BioRad) as described in the General Methods. Maltodextrin (DE4-7, Sigma) was used as the positive control for the enzymes (Table 21).

TABLE 21
Digestibility results for oligosaccharides produced by the
combination of a glucosyltransferase (GTF) and mutanase.

| | | Suc. | Leuc. | Gluc. | Fruc. | digestibility |
|---|---|---|---|---|---|---|
| sample ID | PAA/AMG | (g/L) | (g/L) | (g/L) | (g/L) | (%) |
| GTFJ/mut3264 | no | 0.3 | 0.0 | 0.0 | 0.0 | 1.3 |
| | yes | 0.6 | 0.0 | 0.4 | 0.0 | |
| maltodextrin | no | 0.3 | 0.0 | 0.0 | 0.0 | 91.9 |
| | yes | 0.00 | 0.0 | 25.2 | 0.0 | |

### Example 30

### Production of Oligosaccharides by GTF-S and MUT3264

Reactions comprised sucrose (100 g/L), E. coli crude protein extract containing GTF-S (Streptococcus sp. C150 GI:495810459, GTF0459; Example 9) (10% v/v) in 50 mM phosphate buffer, pH 6.0, or comprised sucrose (100 g/L), E. coli crude protein extract containing GTF-S (Streptococcus sp. C150 GI:495810459, GTF0459; Example 9) (10% v/v) and E. coli crude protein extract containing mut3264 (10% (v/v); Example 12) in 50 mM phosphate buffer, pH 6.0. The total volume for each reaction was 10 mL and all reactions were performed at 37° C. with shaking at 125 rpm. Aliquots were withdrawn at 3, 6, 23 and 26 h and reactions were quenched by heating at 95° C. for 5 min. The insoluble materials were removed by centrifugation and filtration. The filtrate was analyzed by HPLC to determine the concentration of sucrose, glucose, fructose, leucrose and oligosaccharides (Table 22).

TABLE 22
Monosaccharide, disaccharide and oligosaccharide concentrations measured by HPLC.

| | | | | | | | | | | | | | Sum | |
|---|---|---|---|---|---|---|---|---|---|---|---|---|---|---|
| | | Time, | Suc. | Leuc. | Gluc. | Fruc. | DP8+ | DP7 | DP6 | DP5 | DP4 | DP3 | DP3-7 | DP2 |
| Gtf GI | comments | (h) | (g/L) | (g/L) | (g/L) | (g/L) | (g/L) | (g/L) | (g/L) | (g/L) | (g/L) | (g/L) | (g/L) | (g/L) |
| GTF0459 | 10% GTF | 3 | 79.1 | 0.7 | 3.5 | 11.8 | 0.0 | 0.3 | 0.5 | 0.7 | 0.9 | 1.3 | 3.6 | 1.2 |
| | | 6 | 58.3 | 1.9 | 4.3 | 22.0 | 4.6 | 1.9 | 1.9 | 1.8 | 1.7 | 1.9 | 9.2 | 1.9 |
| | | 23 | 8.9 | 5.9 | 4.2 | 44.5 | 17.2 | 4.1 | 3.8 | 3.3 | 2.8 | 2.8 | 16.8 | 2.5 |
| | | 26 | 4.6 | 6.5 | 4.3 | 46.8 | 17.7 | 4.3 | 4.0 | 3.5 | 3.0 | 2.8 | 17.5 | 2.6 |
| GTF0459 | 10% GTF + | 3 | 77.9 | 0.8 | 4.0 | 12.8 | 0.0 | 0.0 | 0.0 | 0.2 | 2.7 | 2.4 | 5.4 | 2.2 |
| | mut3264 | | | | | | | | | | | | | |
| | | 6 | 52.3 | 2.0 | 6.5 | 25.9 | 0.0 | 0.0 | 0.1 | 1.1 | 7.2 | 4.8 | 13.3 | 4.1 |
| | | 23 | 9.4 | 4.9 | 10.1 | 48.3 | 3.8 | 2.1 | 2.2 | 2.0 | 1.8 | 2.1 | 10.2 | 2.2 |
| | | 26 | 9.9 | 4.9 | 10.1 | 48.2 | 0.0 | 0.2 | 0.6 | 1.3 | 13.9 | 10.5 | 26.4 | 10.5 |

### Example 31

### Isolation of Soluble Oligosaccharide Fiber Produced by the Combination of GTF-J and MUT3264

A 200 mL reaction containing 200 g/L sucrose, E. coli concentrated crude protein extract (1.0% v/v) containing GTF-J from S. salivarius (GI:47527, GTF7527; Example 3), and E. coli crude protein extract (10% v/v) containing Paenibacillus humicus mutanase (MUT3264, GI:257153264; Example 12) in distilled, deionized H₂O, was stirred at 30° C. for 20 h, then heated to 90° C. for 15 min to inactivate the enzymes. The resulting product mixture was centrifuged and the resulting supernatant analyzed by HPLC for soluble monosaccharides, disaccharides and oligosaccharides, then 88 mL of the supernatant was purified by SEC using BioGel P2 resin (BioRad). The SEC fractions that contained oligosaccharides≧DP3 were combined and concentrated by rotary evaporation for analysis by HPLC (Table 23).

TABLE 23
Soluble oligosaccharide fiber produced by GTF-J/mut3264.
200 g/L sucrose, GTF-J, mut3264, 30° C., 20 h

| | Product | SEC-purified |
|---|---|---|
| | mixture, | product, |
| | g/L | g/L |
| DP7 | 0 | 0 |
| DP6 | 0 | 0 |
| DP5 | 0 | 0.4 |
| DP4 | 18.0 | 146.9 |
| DP3 | 11.2 | 26.8 |
| DP2 | 10.1 | 0.0 |
| Sucrose | 8.6 | 0.0 |
| Leucrose | 71.4 | 0.0 |
| Glucose | 11.4 | 0.0 |
| Fructose | 68.3 | 0.0 |
| Sum DP2-DP7 | 39.3 | 174.1 |
| Sum DP3-DP7 | 29.2 | 174.1 |

### Example 32

### Isolation of Soluble Oligosaccharide Fiber Produced by the Combination of GTF-L and MUT3264

A 100 mL reaction containing 210 g/L sucrose, E. coli concentrated crude protein extract (10% v/v) containing GTF-L from S. salivarius (GI #662379; Example 5), and E. coli crude protein extract (10% v/v) comprising a Paenibacillus humicus mutanase (MUT3264, GI:257153264; Example 12) in distilled, deionized H₂O, was stirred at 37° C. for 24 h, then heated to 90° C. for 15 min to inactivate the enzymes. The resulting product mixture was centrifuged and the resulting supernatant analyzed by HPLC for soluble monosaccharides, disaccharides and oligosaccharides, then 88 mL of the supernatant was purified by SEC using BioGel P2 resin (BioRad). The SEC fractions that contained oligosaccharides≧DP3 were combined and concentrated by rotary evaporation for analysis by HPLC (Table 24).

TABLE 24
Soluble oligosaccharide fiber produced by GTF-L/mut3264
mutanase.
210 g/L sucrose, GTF-L, mut3264, 37° C., 24 h

| | Product | SEC-purified |
|---|---|---|
| | mixture, | product, |
| | g/L | g/L |
| DP7 | 4.6 | 13.6 |
| DP6 | 6.6 | 16.6 |
| DP5 | 8.0 | 20.5 |
| DP4 | 11.7 | 20.2 |
| DP3 | 12.4 | 5.7 |
| DP2 | 22.0 | 1.1 |
| Sucrose | 10.6 | 0.6 |
| Leucrose | 59.0 | 0.0 |
| Glucose | 12.6 | 0.0 |
| Fructose | 71.5 | 0.0 |
| Sum DP2-DP7 | 65.3 | 77.7 |
| Sum DP3-DP7 | 43.3 | 76.6 |

### Example 33

### Isolation of Soluble Oligosaccharide Fiber Produced by the Combination of GTF-J and MUT3325

A 100 mL reaction containing 210 g/L sucrose, E. coli concentrated crude protein extract (0.6% v/v) containing GTF-J from S. salivarius (GI #47527; Example 3) and T. reesei crude protein extract (20% v/v) comprising a mutanase from Penicillium marneffei ATCC® 18224 (mut3325, GI:212533325; Example 14) in distilled, deionized H₂O, was stirred at 37° C. for 24 h, then heated to 90° C. for 15 min to inactivate the enzymes. The resulting product mixture was centrifuged and the resulting supernatant analyzed by HPLC for soluble monosaccharides, disaccharides and oligosaccharides, then 84 mL of the supernatant was purified by SEC using BioGel P2 resin (BioRad). The SEC fractions that contained oligosaccharides≧DP3 were combined and concentrated by rotary evaporation for analysis by HPLC (Table 25).

TABLE 25
Soluble oligosaccharide fiber produced by GTF-J/mut3325
mutanase.
210 g/L sucrose, GTF-J, mut3325, 37° C., 24 h

| | Product | SEC-purified |
|---|---|---|
| | mixture, | product, |
| | g/L | g/L |
| DP7 | 0.0 | 0.0 |
| DP6 | 0.3 | 0.0 |
| DPS | 14.1 | 60.2 |
| DP4 | 18.8 | 63.9 |
| DP3 | 16.0 | 18.9 |
| DP2 | 3.2 | 0.0 |
| Sucrose | 3.6 | 0.0 |
| Leucrose | 48.6 | 0.0 |
| Glucose | 4.9 | 0.0 |
| Fructose | 78.3 | 0.0 |
| Sum DP2-DP7 | 52.4 | 143.0 |
| Sum DP3-DP7 | 49.2 | 143.0 |

### Example 34

### Isolation of Soluble Oligosaccharide Fiber Produced by the Combination of GTF-I and MUT3325

A 100 mL reaction containing 200 g/L sucrose, E. coli protein crude extract (5% v/v) containing the GTF-I from Streptococcus sobrinus (GI:450874, Example 8) and T. reesei crude protein extract (15% v/v) comprising a mutanase from Penicillium marneffei ATCC® 18224 (MUT3325, GI:212533325; Example 14) in distilled, deionized H₂O, was stirred at 37° C. for 24 h, then heated to 90° C. for 15 min to inactivate the enzymes. The resulting product mixture was centrifuged and the resulting supernatant analyzed by HPLC for soluble monosaccharides, disaccharides and oligosaccharides, then 87 mL of the supernatant was purified by SEC using BioGel P2 resin (BioRad). The SEC fractions that contained oligosaccharides≧DP3 were combined and concentrated by rotary evaporation for analysis by HPLC (Table 26).

TABLE 26
Soluble oligosaccharide fiber produced by GTF-I/mut3325
mutanase.
200 g/L sucrose, GTF-I, mut3325, 37° C., 24 h

| | Product | SEC-purified |
|---|---|---|
| | mixture, | product, |
| | g/L | g/L |
| DP7 | 1.5 | 12.3 |
| DP6 | 4.4 | 16.0 |
| DP5 | 14.5 | 60.5 |
| DP4 | 16.8 | 53.8 |
| DP3 | 12.3 | 15.0 |
| DP2 | 2.3 | 0.0 |
| Sucrose | 4.8 | 0.0 |
| Leucrose | 76.8 | 0.0 |
| Glucose | 6.7 | 0.0 |
| Fructose | 62.3 | 0.2 |
| Sum DP2-DP7 | 51.7 | 157.6 |
| Sum DP3-DP7 | 49.4 | 157.6 |

### Example 35

### Isolation of Soluble Oligosaccharide Fiber Produced by the Combination of GTF-S and MUT3264

A 200 mL reaction containing 210 g/L sucrose, E. coli crude protein extract (10% v/v) containing GTF-S from Streptococcus sp. C150 (GI:495810459; Example 9), and E. coli crude protein extract (10% v/v) comprising a mutanase from Paenibacillus humicus (MUT3264, GI:257153264; Example 12) in distilled, deionized H₂O, was stirred at 37° C. for 40 h, then stored for 84 h at 4° C. prior to heating to 90° C. for 15 min to inactivate the enzymes. The resulting product mixture was centrifuged and the resulting supernatant analyzed by HPLC for soluble monosaccharides, disaccharides and oligosaccharides, then the supernatant was purified by SEC using BioGel P2 resin (BioRad). The SEC fractions that contained oligosaccharides≧DP3 were combined and concentrated by rotary evaporation for analysis by HPLC (Table 27).

TABLE 27
Soluble oligosaccharide fiber produced by GTF-S/mut3264
mutanase.
210 g/L sucrose, GTF-S, mut3264, 37° C., 40 h

| | Product | SEC-purified |
|---|---|---|
| | mixture, | product, |
| | g/L | g/L |
| DP7 | 10.0 | 22.6 |
| DP6 | 12.4 | 42.2 |
| DP5 | 19.4 | 83.3 |
| DP4 | 19.9 | 74.1 |
| DP3 | 13.4 | 22.6 |
| DP2 | 10.4 | 0 |
| Sucrose | 13.4 | 0 |
| Leucrose | 12.7 | 0 |
| Glucose | 8.9 | 0 |
| Fructose | 95.7 | 0 |
| Sum DP2-DP7 | 85.5 | 244.8 |
| Sum DP3-DP7 | 75.1 | 244.8 |

### Example 36

### Isolation of Soluble Oligosaccharide Fiber Produced by the Combination of GTF-B and MUT3264

A 200 mL reaction containing 100 g/L sucrose, E. coli crude protein extract (10% v/v) containing GTF-B from Streptococcus mutans NN2025 (GI:290580544; Example 6), and E. coli crude protein extract (10% v/v) comprising a mutanase from Paenibacillus humicus (MUT3264, GI:257153264; Example 12) in distilled, deionized H₂O, was stirred at 37° C. for 24 h, then heated to 90° C. for 15 min to inactivate the enzymes. The resulting product mixture was centrifuged and the resulting supernatant analyzed by HPLC for soluble monosaccharides, disaccharides and oligosaccharides, then 132 mL of the supernatant was purified by SEC using BioGel P2 resin (BioRad). The SEC fractions that contained oligosaccharides≧DP3 were combined and concentrated by rotary evaporation for analysis by HPLC (Table 28).

TABLE 28
Soluble oligosaccharide fiber produced by GTF-B/mut3264
mutanase.
100 g/L sucrose, GTF-B, mut3264, 37° C., 24 h

| | Product | SEC-purified |
|---|---|---|
| | mixture, | product, |
| | g/L | g/L |
| DP7 | 2.8 | 11.7 |
| DP6 | 4.0 | 14.0 |
| DP5 | 4.3 | 13.2 |
| DP4 | 3.5 | 9.4 |
| DP3 | 4.4 | 2.4 |
| DP2 | 9.8 | 0.0 |
| Sucrose | 10.3 | 0.2 |
| Leucrose | 15.6 | 0.0 |
| Glucose | 2.9 | 0.0 |
| Fructose | 41.7 | 0.1 |
| Sum DP2-DP7 | 28.8 | 50.7 |
| Sum DP3-DP7 | 19.0 | 50.7 |

### Example 37

### Isolation of Soluble Oligosaccharide Fiber Produced by the Combination of GTF-S and MUT3325

A 600 mL reaction containing 300 g/L sucrose, B. subtilis crude protein extract (20% v/v) containing GTF-S from Streptococcus sp. C150 (GI:495810459; Example 11), and T. reesei crude protein extract (2.5% v/v) comprising a mutanase from Penicillium marneffei ATCC® 18224 (MUT3325, GI:212533325; Example 14) in distilled, deionized H₂O, was shaken at 125 rpm and 37° C. for 27.5 h, then heated in a microwave oven (1000 Watts) for 4 min to inactivate the enzymes. The resulting product mixture was centrifuged and the resulting supernatant analyzed by HPLC for soluble monosaccharides, disaccharides and oligosaccharides, then entire supernatant was purified by SEC using BioGel P2 resin (BioRad). The SEC fractions that contained oligosaccharides≧DP3 were combined and concentrated by rotary evaporation for analysis by HPLC (Table 29).

TABLE 29
Soluble oligosaccharide fiber produced by GTF-S/mut3325 mutanase.
300 g/L sucrose, GTF-S, mut3325, 37° C., 24 h

| | Product | SEC-purified |
|---|---|---|
| | mixture, | product, |
| | g/L | g/L |
| DP7 | 4.7 | 10.4 |
| DP6 | 16.4 | 31.1 |
| DP5 | 27.1 | 47.5 |
| DP4 | 30.8 | 38.8 |
| DP3 | 25.6 | 30.5 |
| DP2 | 12.8 | 4.1 |
| Sucrose | 14.0 | 2.5 |
| Leucrose | 18.5 | 0.0 |
| Glucose | 13.0 | 1.4 |
| Fructose | 138.2 | 0.4 |
| Sum DP2-DP7 | 117.5 | 162.4 |
| Sum DP3-DP7 | 104.7 | 158.3 |

### Example 38

### Isolation of Soluble Oligosaccharide Fiber Produced by GTF-J

A 3000 mL reaction containing 200 g/L sucrose and E. coli concentrated crude protein extract (1.0% v/v) containing GTF-J from S. salivarius (GI #47527; Example 3) in distilled, deionized H₂O, was shaken at 125 rpm and pH 5.5 and 47° C. for 21 h, then heated to 60° C. for 30 min to inactivate the enzyme. The resulting product mixture was centrifuged and the resulting supernatant was analyzed by HPLC for soluble monosaccharides, disaccharides and oligosaccharides; the supernatant was then concentrated to 900 mL by rotary evaporation and purified by SEC using BioGel P2 resin (BioRad). The SEC fractions that contained oligosaccharides≧DP3 were combined and concentrated by rotary evaporation for analysis by HPLC (Table 30).

TABLE 30
Soluble oligosaccharide fiber produced by GTF-J.
200 g/L sucrose, GTF-J, 47° C., 24 h

| | Product | SEC-purified |
|---|---|---|
| | mixture, | product, |
| | g/L | g/L |
| DP7 | 0.8 | 2.4 |
| DP6 | 1.5 | 6.5 |
| DP5 | 2.9 | 24.0 |
| DP4 | 4.8 | 26.9 |
| DP3 | 6.5 | 10.7 |
| DP2 | 9.1 | 2.1 |
| Sucrose | 0.7 | 1.5 |
| Leucrose | 55.0 | 0.0 |
| Glucose | 11.9 | 0.3 |
| Fructose | 73.6 | 0.6 |
| Sum DP2-DP7 | 25.6 | 72.6 |
| Sum DP3-DP7 | 16.5 | 70.5 |

### Example 38A

### Isolation of Soluble Oligosaccharide Fiber Produced by the Combination of GTF-S Homolog GTF0974 and MUT3325

A 250 mL reaction containing 450 g/L sucrose, B. subtilis crude protein extract (5% v/v) containing GTF0974 from Streptococcus salivarius 57.I (GI: 387760974; Examples 11A and 11D), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 21 h, then heated to 90° C. for 30 min to inactivate the enzymes. The resulting product mixture was centrifuged and the resulting supernatant analyzed by HPLC for soluble monosaccharides, disaccharides and oligosaccharides (Table 31), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 31). The combined SEC fractions were diluted to 5 wt % dry solids (DS) and freeze-dried to produce the fiber as a dry solid.

TABLE 31
Soluble oligosaccharide fiber produced by GTF0974/mut3325 mutanase.
450 g/L sucrose, GTF0974, mut3325, 47° C., 21 h

| | Product | SEC-purified | SEC-purified |
|---|---|---|---|
| | mixture, | product, | product |
| | g/L | g/L | % (wt/wt DS) |
| DP7+ | 80.6 | 35.5 | 35.3 |
| DP6 | 34.8 | 19.4 | 19.3 |
| DP5 | 37.0 | 17.9 | 17.8 |
| DP4 | 33.7 | 15.7 | 15.6 |
| DP3 | 18.2 | 8.0 | 8.0 |
| DP2 | 12.1 | 1.8 | 1.8 |
| Sucrose | 10.1 | 0.5 | 0.5 |
| Leucrose | 43.4 | 1.7 | 1.7 |
| Glucose | 6.9 | 0.0 | 0.0 |
| Fructose | 200.2 | 0.0 | 0.0 |
| Sum DP2-DP7+ | 216.4 | 98.3 | 97.8 |
| Sum DP3-DP7+ | 204.3 | 96.5 | 96.0 |

### Example 38B

### Isolation of Soluble Oligosaccharide Fiber Produced by the Combination of GTF-S Homolog GTF4336 and MUT3325

A 250 mL reaction containing 450 g/L sucrose, B. subtilis crude protein extract (5% v/v) containing GTF4336 from Streptococcus salivarius SK126 (GI: 488974336; Examples 11A and 11D), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 21 h, then heated to 90° C. for 30 min to inactivate the enzymes. The resulting product mixture was centrifuged and the resulting supernatant analyzed by HPLC for soluble monosaccharides, disaccharides and oligosaccharides (Table 32), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 32). The combined SEC fractions were diluted to 5 wt % dry solids (DS) and freeze-dried to produce the fiber as a dry solid.

TABLE 32
Soluble oligosaccharide fiber produced by GTF4336/mut3325 mutanase.
450 g/L sucrose, GTF4336, mut3325, 47° C., 21 h

| | Product | SEC-purified | SEC-purified |
|---|---|---|---|
| | mixture, | product, | product |
| | g/L | g/L | % (wt/wt DS) |
| DP7+ | 87.0 | 21.0 | 21.6 |
| DP6 | 31.6 | 20.5 | 21.2 |
| DP5 | 29.8 | 23.5 | 24.2 |
| DP4 | 23.4 | 20.8 | 21.4 |
| DP3 | 12.8 | 8.4 | 8.6 |
| DP2 | 8.8 | 2.6 | 2.7 |
| Sucrose | 54.7 | 0.2 | 0.2 |
| Leucrose | 35.3 | 0.1 | 0.1 |
| Glucose | 6.9 | 0.0 | 0.0 |
| Fructose | 182.5 | 0.0 | 0.0 |
| Sum DP2-DP7+ | 193.3 | 96.8 | 99.7 |
| Sum DP3-DP7+ | 184.5 | 94.2 | 97.0 |

### Example 38C

### Isolation of Soluble Oligosaccharide Fiber Produced by the Combination of GTF-S Homolog GTF0470 and MUT3325

A 250 mL reaction containing 450 g/L sucrose, B. subtilis crude protein extract (5% v/v) containing GTF0470 from Streptococcus salivarius K12 (GI: 488980470; Examples 11A and 11D), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 44 h, then heated to 90° C. for 30 min to inactivate the enzymes. The resulting product mixture was centrifuged and the resulting supernatant analyzed by HPLC for soluble monosaccharides, disaccharides and oligosaccharides (Table 33), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 33). The combined SEC fractions were diluted to 5 wt % dry solids (DS) and freeze-dried to produce the fiber as a dry solid.

TABLE 33
Soluble oligosaccharide fiber produced by GTF0470/mut3325 mutanase.
450 g/L sucrose, GTF0470, mut3325, 47° C., 44 h

| | Product | SEC-purified | SEC-purified |
|---|---|---|---|
| | mixture, | product, | product |
| | g/L | g/L | % (wt/wt DS) |
| DP7+ | 48.3 | 29.3 | 27.4 |
| DP6 | 37.5 | 23.6 | 22.0 |
| DP5 | 39.6 | 23.9 | 22.3 |
| DP4 | 36.7 | 19.6 | 18.3 |
| DP3 | 17.2 | 7.7 | 7.2 |
| DP2 | 7.7 | 1.9 | 1.8 |
| Sucrose | 10.1 | 0.5 | 0.5 |
| Leucrose | 40.5 | 0.5 | 0.4 |
| Glucose | 6.8 | 0.0 | 0.0 |
| Fructose | 199.6 | 0.0 | 0.0 |
| Sum DP2-DP7+ | 186.9 | 105.9 | 99.0 |
| Sum DP3-DP7+ | 179.2 | 104.0 | 97.2 |

### Example 38D

### Isolation of Soluble Oligosaccharide Fiber Produced by the Combination of GTF-S Homolog GTF6549 and MUT3325

A 250 mL reaction containing 450 g/L sucrose, B. subtilis crude protein extract (7.5% v/v) containing GTF6549 from Streptococcus salivarius M18 (GI: 490286549; Examples 11A and 11D), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 53 h, then heated to 90° C. for 30 min to inactivate the enzymes. The resulting product mixture was centrifuged and the resulting supernatant analyzed by HPLC for soluble monosaccharides, disaccharides and oligosaccharides (Table 34), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 34). The combined SEC fractions were diluted to 5 wt % dry solids (DS) and freeze-dried to produce the fiber as a dry solid.

TABLE 34
Soluble oligosaccharide fiber produced by GTF6549/mut3325 mutanase.
450 g/L sucrose, GTF6549, mut3325, 47° C., 53 h

| | Product | SEC-purified | SEC-purified |
|---|---|---|---|
| | mixture, | product, | product |
| | g/L | g/L | % (wt/wt DS) |
| DP7+ | 41.9 | 30.1 | 28.4 |
| DP6 | 41.6 | 25.0 | 23.7 |
| DP5 | 41.0 | 22.6 | 21.4 |
| DP4 | 35.9 | 17.9 | 16.9 |
| DP3 | 22.2 | 7.4 | 7.0 |
| DP2 | 10.7 | 1.8 | 1.7 |
| Sucrose | 15.3 | 0.6 | 0.5 |
| Leucrose | 41.2 | 0.3 | 0.3 |
| Glucose | 6.3 | 0.0 | 0.0 |
| Fructose | 193.2 | 0.0 | 0.0 |
| Sum DP2-DP7+ | 193.3 | 104.8 | 99.2 |
| Sum DP3-DP7+ | 182.6 | 103.0 | 97.5 |

### Example 38E

### Isolation of Soluble Oligosaccharide Fiber Produced by the Combination of GTF-S Homolog GTF4491 and MUT3325

A 250 mL reaction containing 450 g/L sucrose, B. subtilis crude protein extract (5% v/v) containing GTF4491 from Streptococcus salivarius JIM8777 (GI: 387784491; Examples 11A and 11D), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 22 h, then heated to 90° C. for 30 min to inactivate the enzymes. The resulting product mixture was centrifuged and the resulting supernatant analyzed by HPLC for soluble monosaccharides, disaccharides and oligosaccharides (Table 35), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 35). The combined SEC fractions were diluted to 5 wt % dry solids (DS) and freeze-dried to produce the fiber as a dry solid.

TABLE 35
Soluble oligosaccharide fiber produced by GTF4491/mut3325 mutanase.
450 g/L sucrose, GTF4491, mut3325, 47° C., 22 h

| | Product | SEC-purified | SEC-purified |
|---|---|---|---|
| | mixture, | product, | product |
| | g/L | g/L | % (wt/wt DS) |
| DP7+ | 89.7 | 46.9 | 44.5 |
| DP6 | 30.8 | 18.3 | 17.4 |
| DP5 | 29.2 | 18.2 | 17.3 |
| DP4 | 23.1 | 13.7 | 13.0 |
| DP3 | 11.5 | 5.2 | 4.9 |
| DP2 | 7.4 | 1.8 | 1.7 |
| Sucrose | 17.1 | 0.6 | 0.6 |
| Leucrose | 35.7 | 0.5 | 0.5 |
| Glucose | 8.7 | 0.0 | 0.0 |
| Fructose | 186.3 | 0.0 | 0.0 |
| Sum DP2-DP7+ | 191.6 | 104.1 | 98.9 |
| Sum DP3-DP7+ | 184.2 | 102.3 | 97.2 |

### Example 38F

### Isolation of Soluble Oligosaccharide Fiber Produced by the Combination of GTF-S Homolog GTF1645 and MUT3325

A 250 mL reaction containing 450 g/L sucrose, B. subtilis crude protein extract (5% v/v) containing GTF1645 from Streptococcus sp. HSISS3 (GI: 544721645; Example 11A), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 46 h, then heated to 90° C. for 30 min to inactivate the enzymes. The resulting product mixture was centrifuged and the resulting supernatant analyzed by HPLC for soluble monosaccharides, disaccharides and oligosaccharides (Table 36), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 36). The combined SEC fractions were diluted to 5 wt % dry solids (DS) and freeze-dried to produce the fiber as a dry solid.

TABLE 36
Soluble oligosaccharide fiber produced by GTF1645/mut3325 mutanase.
450 g/L sucrose, GTF1645, mut3325, 47° C., 46 h

| | Product | SEC-purified | SEC-purified |
|---|---|---|---|
| | mixture, | product, | product |
| | g/L | g/L | % (wt/wt DS) |
| DP7+ | 0.0 | 15.7 | 15.3 |
| DP6 | 50.8 | 24.7 | 24.2 |
| DP5 | 39.2 | 24.9 | 24.4 |
| DP4 | 39.6 | 23.2 | 22.7 |
| DP3 | 29.8 | 10.6 | 10.4 |
| DP2 | 11.7 | 2.2 | 2.1 |
| Sucrose | 14.3 | 0.6 | 0.6 |
| Leucrose | 30.1 | 0.2 | 0.2 |
| Glucose | 8.2 | 0.0 | 0.0 |
| Fructose | 192.6 | 0.0 | 0.0 |
| Sum DP2-DP7+ | 171.0 | 101.2 | 99.2 |
| Sum DP3-DP7+ | 159.3 | 99.0 | 97.1 |

### Example 38G

### Isolation of Soluble Oligosaccharide Fiber Produced by the Combination of GTF-S Homolog GTF6099 and MUT3325

A 250 mL reaction containing 450 g/L sucrose, B. subtilis crude protein extract (5% v/v) containing GTF6099 from Streptococcus sp. HSISS2 (GI: 544716099; Example 11A), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 52 h, then heated to 90° C. for 30 min to inactivate the enzymes. The resulting product mixture was centrifuged and the resulting supernatant analyzed by HPLC for soluble monosaccharides, disaccharides and oligosaccharides (Table 37), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 37). The combined SEC fractions were diluted to 5 wt % dry solids (DS) and freeze-dried to produce the fiber as a dry solid.

TABLE 37
Soluble oligosaccharide fiber produced by GTF6099/mut3325 mutanase.
450 g/L sucrose, GTF6099, mut3325, 47° C., 52 h

| | Product | SEC-purified | SEC-purified |
|---|---|---|---|
| | mixture, | product, | product |
| | g/L | g/L | % (wt/wt DS) |
| DP7+ | 0.0 | 16.1 | 16.0 |
| DP6 | 57.0 | 23.7 | 23.5 |
| DP5 | 43.9 | 26.3 | 26.1 |
| DP4 | 42.7 | 22.1 | 21.9 |
| DP3 | 29.1 | 9.7 | 9.6 |
| DP2 | 11.9 | 2.1 | 2.1 |
| Sucrose | 15.7 | 0.5 | 0.5 |
| Leucrose | 34.4 | 0.2 | 0.2 |
| Glucose | 7.6 | 0.0 | 0.0 |
| Fructose | 190.9 | 0.0 | 0.0 |
| Sum DP2-DP7+ | 184.6 | 99.9 | 99.3 |
| Sum DP3-DP7+ | 172.8 | 97.8 | 97.2 |

### Example 38H

### Isolation of Soluble Oligosaccharide Fiber Produced by the Combination of GTF-S Homolog GTF7317 and MUT3325

A 250 mL reaction containing 450 g/L sucrose, B. subtilis crude protein extract (5% v/v) containing GTF7317 from Streptococcus salivarius PS4 (GI: 488977317; Example 11A), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 46 h, then heated to 90° C. for 30 min to inactivate the enzymes. The resulting product mixture was centrifuged and the resulting supernatant analyzed by HPLC for soluble monosaccharides, disaccharides and oligosaccharides (Table 38), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 38). The combined SEC fractions were diluted to 5 wt % dry solids (DS) and freeze-dried to produce the fiber as a dry solid.

TABLE 38
Soluble oligosaccharide fiber produced by GTF7317/mut3325 mutanase.
450 g/L sucrose, GTF7317, mut3325, 47° C., 46 h

| | Product | SEC-purified | SEC-purified |
|---|---|---|---|
| | mixture, | product, | product |
| | g/L | g/L | % (wt/wt DS) |
| DP7+ | 0.0 | 16.5 | 16.0 |
| DP6 | 57.1 | 23.0 | 22.4 |
| DP5 | 43.7 | 25.8 | 25.2 |
| DP4 | 42.6 | 23.2 | 22.6 |
| DP3 | 28.7 | 11.0 | 10.7 |
| DP2 | 11.6 | 2.3 | 2.2 |
| Sucrose | 13.8 | 0.6 | 0.6 |
| Leucrose | 35.8 | 0.3 | 0.3 |
| Glucose | 6.9 | 0.0 | 0.0 |
| Fructose | 192.5 | 0.0 | 0.0 |
| Sum DP2-DP7+ | 183.6 | 101.6 | 99.1 |
| Sum DP3-DP7+ | 172.0 | 99.3 | 96.9 |

### Example 38I

### Isolation of Soluble Oligosaccharide Fiber Produced by the Combination of GTF-S Homolog GTF8487 and MUT3325

A 250 mL reaction containing 450 g/L sucrose, B. subtilis crude protein extract (5% v/v) containing GTF8487 from Streptococcus salivarius CCHSS3 (GI: 340398487; Example 11A), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 40 h, then heated to 90° C. for 30 min to inactivate the enzymes. The resulting product mixture was centrifuged and the resulting supernatant analyzed by HPLC for soluble monosaccharides, disaccharides and oligosaccharides (Table 39), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 39). The combined SEC fractions were diluted to 5 wt % dry solids (DS) and freeze-dried to produce the fiber as a dry solid.

TABLE 39
Soluble oligosaccharide fiber produced by GTF8487/mut3325 mutanase.
450 g/L sucrose, GTF8487, mut3325, 47° C., 40 h

| | Product | SEC-purified | SEC-purified |
|---|---|---|---|
| | mixture, | product, | product |
| | g/L | g/L | % (wt/wt DS) |
| DP7+ | 75.3 | 41.9 | 39.1 |
| DP6 | 33.3 | 19.5 | 18.2 |
| DP5 | 34.8 | 19.7 | 18.4 |
| DP4 | 30.0 | 16.0 | 15.0 |
| DP3 | 13.9 | 6.3 | 5.8 |
| DP2 | 8.2 | 2.1 | 2.0 |
| Sucrose | 10.1 | 0.6 | 0.6 |
| Leucrose | 46.0 | 1.0 | 0.9 |
| Glucose | 6.9 | 0.0 | 0.0 |
| Fructose | 197.8 | 0.0 | 0.0 |
| Sum DP2-DP7+ | 195.5 | 105.5 | 98.5 |
| Sum DP3-DP7+ | 187.3 | 103.4 | 96.5 |

### Example 38J

### Isolation of Soluble Oligosaccharide Fiber Produced by the Combination of GTF-S Homolog GTF3879 and MUT3325

A 250 mL reaction containing 450 g/L sucrose, B. subtilis crude protein extract (15% v/v) containing GTF3879 from Streptococcus sp. HSISS4 (GI: 544713879; Example 11A), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 52 h, then heated to 90° C. for 30 min to inactivate the enzymes. The resulting product mixture was centrifuged and the resulting supernatant analyzed by HPLC for soluble monosaccharides, disaccharides and oligosaccharides (Table 40), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 40). The combined SEC fractions were diluted to 5 wt % dry solids (DS) and freeze-dried to produce the fiber as a dry solid.

TABLE 40
Soluble oligosaccharide fiber produced by GTF3879/mut3325 mutanase.
450 g/L sucrose, GTF3879, mut3325, 47° C., 52 h

| | Product | SEC-purified | SEC-purified |
|---|---|---|---|
| | mixture, | product, | product |
| | g/L | g/L | % (wt/wt DS) |
| DP7+ | 31.8 | 23.4 | 22.4 |
| DP6 | 41.3 | 25.6 | 24.4 |
| DP5 | 40.8 | 23.7 | 22.5 |
| DP4 | 36.3 | 19.3 | 18.4 |
| DP3 | 19.9 | 8.8 | 8.4 |
| DP2 | 8.5 | 2.2 | 2.1 |
| Sucrose | 20.8 | 1.1 | 1.1 |
| Leucrose | 37.0 | 0.7 | 0.7 |
| Glucose | 6.8 | 0.0 | 0.0 |
| Fructose | 188.3 | 0.0 | 0.0 |
| Sum DP2-DP7+ | 178.6 | 103.0 | 98.2 |
| Sum DP3-DP7+ | 170.1 | 100.8 | 96.1 |

### Example 38K

### Isolation of Soluble Oligosaccharide Fiber Produced by the Combination of GTF-S Homolog GTF3808 and MUT3325

A 250 mL reaction containing 450 g/L sucrose, B. subtilis crude protein extract (5% v/v) containing GTF3808 from Streptococcus sp. SR4 (GI: 573493808; Example 11A), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 22 h, then heated to 90° C. for 30 min to inactivate the enzymes. The resulting product mixture was centrifuged and the resulting supernatant analyzed by HPLC for soluble monosaccharides, disaccharides and oligosaccharides (Table 41), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 41). The combined SEC fractions were diluted to 5 wt % dry solids (DS) and freeze-dried to produce the fiber as a dry solid.

TABLE 41
Soluble oligosaccharide fiber produced by GTF3808/mut3325 mutanase.
450 g/L sucrose, GTF3808, mut3325, 47° C., 22 h

| | Product | SEC-purified | SEC-purified |
|---|---|---|---|
| | mixture, | product, | product |
| | g/L | g/L | % (wt/wt DS) |
| DP7+ | 26.2 | 10.8 | 9.8 |
| DP6 | 31.2 | 19.9 | 18.0 |
| DP5 | 39.0 | 25.9 | 23.5 |
| DP4 | 39.4 | 22.5 | 20.4 |
| DP3 | 27.1 | 10.5 | 9.5 |
| DP2 | 15.5 | 2.4 | 2.2 |
| Sucrose | 15.6 | 0.5 | 0.5 |
| Leucrose | 51.1 | 0.3 | 0.3 |
| Glucose | 6.6 | 0.0 | 0.0 |
| Fructose | 195.1 | 0.0 | 0.0 |
| Sum DP2-DP7+ | 178.4 | 109.3 | 99.2 |
| Sum DP3-DP7+ | 162.9 | 106.9 | 97.0 |

### Example 38L

### Isolation of Soluble Oligosaccharide Fiber Produced by the Combination of GTF-S Homolog GTF8467 and MUT3325

A 250 mL reaction containing 450 g/L sucrose, B. subtilis crude protein extract (5% v/v) containing GTF8467 from Streptococcus salivarius NU10 (GI: 660358467; Example 11A), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 47 h, then heated to 90° C. for 30 min to inactivate the enzymes. The resulting product mixture was centrifuged and the resulting supernatant analyzed by HPLC for soluble monosaccharides, disaccharides and oligosaccharides (Table 42), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 42). The combined SEC fractions were diluted to 5 wt % dry solids (DS) and freeze-dried to produce the fiber as a dry solid.

TABLE 42
Soluble oligosaccharide fiber produced by GTF8467/mut3325 mutanase.
450 g/L sucrose, GTF8467, mut3325, 47° C., 47 h

| | Product | SEC-purified | SEC-purified |
|---|---|---|---|
| | mixture, | product, | product |
| | g/L | g/L | % (wt/wt DS) |
| DP7+ | 0.0 | 11.1 | 10.5 |
| DP6 | 57.0 | 20.5 | 19.6 |
| DP5 | 37.8 | 30.1 | 28.7 |
| DP4 | 34.3 | 27.2 | 25.9 |
| DP3 | 20.3 | 12.8 | 12.2 |
| DP2 | 7.5 | 2.5 | 2.4 |
| Sucrose | 69.6 | 0.4 | 0.4 |
| Leucrose | 34.0 | 0.2 | 0.2 |
| Glucose | 6.3 | 0.0 | 0.0 |
| Fructose | 178.3 | 0.0 | 0.0 |
| Sum DP2-DP7+ | 156.8 | 104.1 | 99.5 |
| Sum DP3-DP7+ | 149.3 | 101.6 | 97.1 |

### Example 38M

### Isolation of Soluble Oligosaccharide Fiber Produced by the Combination of GTF-S Homolog GTF0060 and MUT3325

A 250 mL reaction containing 450 g/L sucrose, B. subtilis crude protein extract (5% v/v) containing GTF0060 from Streptococcus sp. ACS2 (GI: 576980060; Example 11A), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 47 h, then heated to 90° C. for 30 min to inactivate the enzymes. The resulting product mixture was centrifuged and the resulting supernatant analyzed by HPLC for soluble monosaccharides, disaccharides and oligosaccharides (Table 43), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 43). The combined SEC fractions were diluted to 5 wt % dry solids (DS) and freeze-dried to produce the fiber as a dry solid.

TABLE 43
Soluble oligosaccharide fiber produced by GTF0060/mut3325 mutanase.
450 g/L sucrose, GTF0060, mut3325, 47° C., 47 h

| | Product | SEC-purified | SEC-purified |
|---|---|---|---|
| | mixture, | product, | product |
| | g/L | g/L | % (wt/wt DS) |
| DP7+ | 27.7 | 19.1 | 17.2 |
| DP6 | 41.7 | 28.6 | 27.2 |
| DP5 | 41.9 | 25.8 | 24.5 |
| DP4 | 37.7 | 21.0 | 20.0 |
| DP3 | 22.0 | 9.0 | 8.6 |
| DP2 | 8.4 | 1.9 | 1.8 |
| Sucrose | 23.1 | 0.5 | 0.5 |
| Leucrose | 39.1 | 0.3 | 0.3 |
| Glucose | 5.6 | 0.0 | 0.0 |
| Fructose | 198.6 | 0.0 | 0.0 |
| Sum DP2-DP7+ | 179.5 | 104.4 | 99.3 |
| Sum DP3-DP7+ | 171.1 | 102.5 | 97.5 |

### Comparative Example 38N

### Isolation of Soluble Oligosaccharide Fiber Produced by the Combination of GTF-S and MUT3325

A 250 mL reaction containing 450 g/L sucrose, B. subtilis crude protein extract (5% v/v) containing GTF0459 from Streptococcus sp. C150 (GI: 495810459; Examples 11A and 11C), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 90 h, then heated to 90° C. for 30 min to inactivate the enzymes. The resulting product mixture was centrifuged and the resulting supernatant analyzed by HPLC for soluble monosaccharides, disaccharides and oligosaccharides (Table 44), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 44). The combined SEC fractions were diluted to 5 wt % dry solids (DS) and freeze-dried to produce the fiber as a dry solid.

TABLE 44
Soluble oligosaccharide fiber produced
by GTF0459/mut3325 mutanase.
450 g/L sucrose, GTF0459, mut3325, 47° C., 90 h

| | Product | SEC-purified | SEC-purified |
|---|---|---|---|
| | mixture, | product, | product |
| | g/L | g/L | % (wt/wt DS) |
| DP7+ | 24.2 | 29.0 | 27.0 |
| DP6 | 41.2 | 21.5 | 20.0 |
| DP5 | 45.0 | 24.2 | 22.5 |
| DP4 | 40.8 | 20.5 | 19.0 |
| DP3 | 25.7 | 9.4 | 8.7 |
| DP2 | 10.3 | 2.1 | 1.9 |
| Sucrose | 24.1 | 0.5 | 0.5 |
| Leucrose | 35.9 | 0.4 | 0.3 |
| Glucose | 6.9 | 0.0 | 0.0 |
| Fructose | 198.6 | 0.0 | 0.0 |
| Sum DP2-DP7+ | 197.6 | 106.7 | 99.2 |
| Sum DP3-DP7+ | 187.3 | 104.6 | 97.3 |

### Comparative Example 38O

### Isolation of Soluble Oligosaccharide Fiber Produced by the Combination of GTF-S Non-Homolog GTF0487 and MUT3325

A 250 mL reaction containing 450 g/L sucrose, B. subtilis crude protein extract (20% v/v) containing GTF0487 from Streptococcus salivarius PS4 (GI: 495810487; Examples 11A and 11C), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 214 h, then heated to 90° C. for 30 min to inactivate the enzymes. The resulting product mixture was centrifuged and the resulting supernatant analyzed by HPLC for soluble monosaccharides, disaccharides and oligosaccharides (Table 45), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 45). The combined SEC fractions were diluted to 5 wt % dry solids (DS) and freeze-dried to produce the fiber as a dry solid.

TABLE 45
Soluble oligosaccharide fiber produced
by GTF0487/mut3325 mutanase.
450 g/L sucrose, GTF0487, mut0487, 47° C., 214 h

| | Product | SEC-purified | SEC-purified |
|---|---|---|---|
| | mixture, | product, | product |
| | g/L | g/L | % (wt/wt DS) |
| DP7+ | 6.0 | 21.6 | 30.4 |
| DP6 | 3.9 | 10.2 | 14.4 |
| DP5 | 7.9 | 15.9 | 22.3 |
| DP4 | 9.1 | 13.3 | 18.6 |
| DP3 | 8.2 | 6.3 | 8.8 |
| DP2 | 8.6 | 2.4 | 3.3 |
| Sucrose | 96.9 | 0.6 | 0.9 |
| Leucrose | 18.0 | 0.1 | 0.1 |
| Glucose | 94.9 | 0.2 | 0.3 |
| Fructose | 106.0 | 0.7 | 1.0 |
| Sum DP2-DP7+ | 43.7 | 69.7 | 97.8 |
| Sum DP3-DP7+ | 35.1 | 67.3 | 94.5 |

### Comparative Example 38P

### Isolation of Soluble Oligosaccharide Fiber Produced by the Combination of GTF-S Non-Homolog GTF5360 and MUT3325

A 250 mL reaction containing 450 g/L sucrose, B. subtilis crude protein extract (20% v/v) containing GTF5360 from Streptococcus mutans JP9-4 (GI: 440355360; Examples 11A and 11C), and T. reesei crude protein extract UFC (0.075% v/v) comprising a mutanase from Penicillium marneffei ATCC® 18224 (MUT3325, GI:212533325; Example 15) in distilled, deionized H₂O, was stirred at pH 5.5 and 47° C. for 214 h, then heated to 90° C. for 30 min to inactivate the enzymes. The resulting product mixture was centrifuged and the resulting supernatant analyzed by HPLC for soluble monosaccharides, disaccharides and oligosaccharides (Table 46), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 46). The combined SEC fractions were diluted to 5 wt % dry solids (DS) and freeze-dried to produce the fiber as a dry solid.

TABLE 46
Soluble oligosaccharide fiber produced
by GTF5360/mut3325 mutanase.
450 g/L sucrose, GTF5360, mut3325, 47° C., 214 h

| | Product | SEC-purified | SEC-purified |
|---|---|---|---|
| | mixture, | product, | product |
| | g/L | g/L | % (wt/wt DS) |
| DP7+ | 33.2 | 48.9 | 46.4 |
| DP6 | 15.1 | 17.7 | 16.8 |
| DP5 | 19.2 | 19.9 | 18.9 |
| DP4 | 16.2 | 11.9 | 11.3 |
| DP3 | 11.2 | 5.0 | 4.8 |
| DP2 | 10.7 | 1.8 | 1.7 |
| Sucrose | 29.5 | 0.2 | 0.2 |
| Leucrose | 56.9 | 0.1 | 0.1 |
| Glucose | 53.5 | 0.0 | 0.0 |
| Fructose | 145.9 | 0.0 | 0.0 |
| Sum DP2-DP7+ | 105.5 | 105.3 | 99.8 |
| Sum DP3-DP7+ | 94.8 | 103.5 | 98.1 |

### Example 38Q

### Isolation of Soluble Oligosaccharide Fiber Produced by the Combination of C-Terminal Truncated GTF0974-T4 and MUT3325

A 250 mL reaction containing 450 g/L sucrose, B. subtilis crude protein extract (0.61% v/v) containing a version of GTF0974 from Streptococcus salivarius 57.I (GI: 387760974; Examples 11A and 11C) having additional C terminal truncations of part of the glucan binding domains (GTF0974-T4, Example 11B), and T. reesei crude protein extract UFC (0.11% v/v) comprising a mutanase from Penicillium marneffei ATCC® 18224 (MUT3325, GI:212533325; Example 15) in distilled, deionized H₂O, was stirred at pH 5.5 and 47° C. for 24 h, then heated to 90° C. for 30 min to inactivate the enzymes. The resulting product mixture was centrifuged and the resulting supernatant analyzed by HPLC for soluble monosaccharides, disaccharides and oligosaccharides (Table 47), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 47). The combined SEC fractions were diluted to 5 wt % dry solids (DS) and freeze-dried to produce the fiber as a dry solid.

TABLE 47
Soluble oligosaccharide fiber produced
by GTF0974-T4/mut3325 mutanase.
450 g/L sucrose, GTF0974-T4, mut3325, 47° C., 24 h

| | Product | SEC-purified | SEC-purified |
|---|---|---|---|
| | mixture, | product, | product |
| | g/L | g/L | % (wt/wt DS) |
| DP7+ | 47.6 | 29.0 | 26.7 |
| DP6 | 41.7 | 25.5 | 23.5 |
| DP5 | 44.4 | 25.6 | 23.6 |
| DP4 | 41.2 | 19.4 | 17.8 |
| DP3 | 23.8 | 7.5 | 6.9 |
| DP2 | 12.0 | 1.7 | 1.5 |
| Sucrose | 11.0 | 0.0 | 0.0 |
| Leucrose | 42.0 | 0.0 | 0.0 |
| Glucose | 6.2 | 0.0 | 0.0 |
| Fructose | 200.6 | 0.0 | 0.0 |
| Sum DP2-DP7+ | 210.7 | 108.7 | 100 |
| Sum DP3-DP7+ | 198.7 | 107.0 | 98.5 |

### Example 38R

### Isolation of Soluble Oligosaccharide Fiber Produced by the Combination of C-Terminal Truncated GTF0974-T5 and MUT3325

A 250 mL reaction containing 450 g/L sucrose, B. subtilis crude protein extract (0.51% v/v) containing a version of GTF0974 from Streptococcus salivarius 57.I (GI: 387760974; Examples 11A and 11C) having additional C terminal truncations of part of the glucan binding domains (GTF0974-T5, Example 11B), and T. reesei crude protein extract UFC (0.11% v/v) comprising a mutanase from Penicillium marneffei ATCC® 18224 (MUT3325, GI:212533325; Example 15) in distilled, deionized H₂O, was stirred at pH 5.5 and 47° C. for 24 h, then heated to 90° C. for 30 min to inactivate the enzymes. The resulting product mixture was centrifuged and the resulting supernatant analyzed by HPLC for soluble monosaccharides, disaccharides and oligosaccharides (Table 48), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 48). The combined SEC fractions were diluted to 5 wt % dry solids (DS) and freeze-dried to produce the fiber as a dry solid.

TABLE 48
Soluble oligosaccharide fiber produced
by GTF0974-T5/mut3325 mutanase.
450 g/L sucrose, GTF0974-T5, mut3325, 47° C., 24 h

| | Product | SEC-purified | SEC-purified |
|---|---|---|---|
| | mixture, | product, | product |
| | g/L | g/L | % (wt/wt DS) |
| DP7+ | 41.0 | 23.9 | 22.2 |
| DP6 | 42.7 | 26.9 | 25.0 |
| DP5 | 44.5 | 27.2 | 25.2 |
| DP4 | 40.3 | 20.6 | 19.1 |
| DP3 | 24.2 | 7.9 | 7.3 |
| DP2 | 11.5 | 1.3 | 1.2 |
| Sucrose | 12.3 | 0.0 | 0.0 |
| Leucrose | 42.0 | 0.0 | 0.0 |
| Glucose | 6.0 | 0.0 | 0.0 |
| Fructose | 201.9 | 0.0 | 0.0 |
| Sum DP2-DP7+ | 204.2 | 107.8 | 100 |
| Sum DP3-DP7+ | 192.7 | 106.5 | 98.8 |

### Example 38S

### Isolation of Soluble Oligosaccharide Fiber Produced by the Combination of C-Terminal Truncated GTF3808-T5 and MUT3325

A 250 mL reaction containing 450 g/L sucrose, B. subtilis crude protein extract (0.77% v/v) containing a version of GTF3808 from Streptococcus sp. SR4 (GI: 573493808; Examples 11A and 11C) having additional C terminal truncations of part of the glucan binding domains (GTF3808-T5, Example 11B), and T. reesei crude protein extract UFC (0.11% v/v) comprising a mutanase from Penicillium marneffei ATCC® 18224 (MUT3325, GI:212533325; Example 15) in distilled, deionized H₂O, was stirred at pH 5.5 and 47° C. for 19 h, then heated to 90° C. for 30 min to inactivate the enzymes. The resulting product mixture was centrifuged and the resulting supernatant analyzed by HPLC for soluble monosaccharides, disaccharides and oligosaccharides (Table 49), then the oligosaccharides were isolated from the supernatant by SEC at 40° C. using Diaion UBK 530 (Na+ form) resin (Mitsubishi). The SEC fractions that contained oligosaccharides≧DP3 were combined and concentrated by rotary evaporation for analysis by HPLC (Table 49). The combined SEC fractions were diluted to 5 wt % dry solids (DS) and freeze-dried to produce the fiber as a dry solid.

TABLE 49
Soluble oligosaccharide fiber produced
by GTF3808-T5/mut3325 mutanase.
450 g/L sucrose, GTF3808-T5, mut3325, 47° C., 19 h

| | Product | SEC-purified | SEC-purified |
|---|---|---|---|
| | mixture, | product, | product |
| | g/L | g/L | % (wt/wt DS) |
| DP7+ | 55.7 | 29.2 | 26.5 |
| DP6 | 38.7 | 23.8 | 21.7 |
| DP5 | 42.4 | 25.1 | 22.9 |
| DP4 | 39.3 | 20.5 | 18.7 |
| DP3 | 21.5 | 8.1 | 7.4 |
| DP2 | 11.8 | 1.6 | 1.5 |
| Sucrose | 10.9 | 0.5 | 0.5 |
| Leucrose | 41.6 | 0.1 | 0.1 |
| Glucose | 6.3 | 0.0 | 0.0 |
| Fructose | 196.1 | 0.0 | 0.0 |
| Sum DP2-DP7+ | 209.3 | 108.3 | 99.4 |
| Sum DP3-DP7+ | 197.6 | 106.7 | 97.9 |

### Example 39

### Anomeric Linkage Analysis of Soluble Oligosaccharide Fiber Produced by GTF-J and by GTF/Mutanase Combinations

Solutions of chromatographically-purified soluble oligosaccharide fibers prepared as described in Examples 31 to Example 38 were dried to a constant weight by lyophilization, and the resulting solids analyzed by ¹H NMR spectroscopy and by GC/MS as described in the General Methods section (above). The anomeric linkages for each of these soluble oligosaccharide fiber mixtures are reported in Tables 50 and 51.

TABLE 50
Anomeric linkage analysis of soluble oligosaccharides
by ¹H NMR spectroscopy.

| | | % | % | % | % |
|---|---|---|---|---|---|
| Example | | α- | α- | α- | α- |
| # | GTF/mutanase | (1, 3) | (1, 3, 6) | (1, 2, 6) | (1, 6) |
| 31 | GTF 7527/mut3264 | 89.6 | 1.8 | 0.0 | 8.6 |
| 32 | GTF 2379/mut3264 | 60.2 | 3.3 | 0.0 | 36.6 |
| 33 | GTF 7527/mut3325 | 95.2 | 2.0 | 0.0 | 2.8 |
| 34 | GTF 0874/mut3325 | 75.2 | 0.0 | 0.0 | 24.8 |
| 35 | GTF 0459/mut3264 | 88.2 | 5.7 | 0.0 | 6.1 |
| 36 | GTF 0544/mut3264 | 15.0 | 3.4 | 0.0 | 81.6 |
| 37 | GTF 0459/mut3325 | 88.9 | 5.7 | 0.0 | 5.4 |
| 38 | GTF 7527/no mutanase | 74.6 | 9.8 | 0.0 | 15.6 |

TABLE 51
Anomeric linkage analysis of soluble oligosaccharides by GC/MS.

| | | | | | | | | % | % | % |
|---|---|---|---|---|---|---|---|---|---|---|
| | | % | % | % | % | % | % | α- | α- | α-(1,4,6) + α- |
| Example # | GTF/mutanase | α-(1,4) | α-(1,3) | α-(1,3,6) | 2,1 Fruc | α-(1,2) | α-(1,6) | (1,3,4) | (1,2,3) | (1,2,6) |
| 33 | GTF 7527/mut3325 | 0.4 | 97.1 | 0.6 | 0.0 | 0.6 | 0.9 | 0.1 | 0.2 | 0.1 |
| 35 | GTF 0459/mut3264 | 0.4 | 96.9 | 1.4 | 0.0 | 0.2 | 0.7 | 0.1 | 0.2 | 0.0 |
| 36 | GTF 0544/mut3264 | 0.4 | 24.1 | 2.5 | 1.0 | 0.5 | 70.9 | 0.0 | 0.0 | 0.6 |
| 37 | GTF 0459/mut3325 | 0.5 | 95.0 | 1.7 | 1.1 | 0.5 | 0.9 | 0.0 | 0.0 | 0.2 |
| 38 | GTF 7527/no | 0.9 | 90.8 | 2.2 | 0.0 | 0.4 | 5.0 | 0.1 | 0.4 | 0.2 |
| | mutanase | | | | | | | | | |

### Example 39A

### Anomeric Linkage Analysis of Soluble Oligosaccharide Fiber Produced by GTF-S, GTF-S Homologs and GTF-S Non-Homologs in Combination with MUT3325

Solutions of chromatographically-purified soluble oligosaccharide fibers prepared as described in Examples 38A to Example 38P were dried to a constant weight by lyophilization, and the resulting solids analyzed by ¹H NMR spectroscopy and by GC/MS as described in the General Methods section (above). The anomeric linkages for each of these soluble oligosaccharide fiber mixtures are reported in Tables 52 and 53.

TABLE 52
Anomeric linkage analysis of soluble oligosaccharides
by ¹H NMR spectroscopy.

| | | % | % | % | % |
|---|---|---|---|---|---|
| Example | | α- | α- | α- | α- |
| # | GTF/mutanase | (1, 3) | (1, 3, 6) | (1, 2, 6) | (1, 6) |
| 38A | GTF0974/mut3325 | 94.0 | 1.0 | 0.0 | 5.0 |
| 38B | GTF4336/mut3325 | 93.9 | 1.0 | 0.0 | 5.1 |
| 38C | GTF0470/mut3325 | 94.2 | 1.1 | 0.0 | 4.7 |
| 38D | GTF6549/mut3325 | 93.4 | 1.2 | 0.0 | 5.4 |
| 38E | GTF4491/mut3325 | 94.3 | 1.1 | 0.0 | 4.6 |
| 38F | GTF1645/mut3325 | 93.2 | 1.4 | 0.0 | 5.4 |
| 38G | GTF6099/mut3325 | 93.2 | 1.4 | 0.0 | 5.4 |
| 38H | GTF7317/mut3325 | 92.7 | 1.5 | 0.0 | 5.8 |
| 38I | GTF8487/mut3325 | 94.1 | 1.0 | 0.0 | 4.8 |
| 38J | GTF3879/mut3325 | 95.2 | 0.0 | 0.0 | 4.8 |
| 38K | GTF3808/mut3325 | 93.4 | 0.0 | 0.0 | 6.6 |
| 38L | GTF8467/mut3325 | 95.2 | 0.0 | 0.0 | 4.8 |
| 38M | GTF0060/mut3325 | 94.7 | 0.0 | 0.0 | 5.3 |
| 38N | GTF0479/mut3325 | 94.4 | 0.0 | 0.0 | 5.6 |
| 38O | GTF0487/mut3325 | 27.2 | 2.2 | 0.0 | 70.5 |
| 38P | GTF5360/mut3325 | 19.9 | 1.5 | 0.0 | 78.6 |

TABLE 53
Anomeric linkage analysis of soluble oligosaccharides by GC/MS.

| | | | | | | | % | % | % |
|---|---|---|---|---|---|---|---|---|---|
| | | % | % | % | % | % | α- | α- | α-(1,4,6) + α- |
| Example # | GTF/mutanase | α-(1,4) | α-(1,3) | α-(1,3,6) | α-(1,2) | α-(1,6) | (1,3,4) | (1,2,3) | (1,2,6) |
| 38A | GTF0974/mut3325 | 0.6 | 96.0 | 1.5 | 0.2 | 1.1 | 0.2 | 0.4 | 0.0 |
| 38B | GTF4336/mut3325 | 0.8 | 94.8 | 2.1 | 0.2 | 1.3 | 0.3 | 0.5 | 0.0 |
| 38C | GTF0470/mut3325 | 0.3 | 96.9 | 1.4 | 0.1 | 0.8 | 0.0 | 0.4 | 0.0 |
| 38D | GTF6549/mut3325 | 0.5 | 96.7 | 1.5 | 0.1 | 0.8 | 0.0 | 0.4 | 0.0 |
| 38E | GTF4491/mut3325 | 0.4 | 96.9 | 1.2 | 0.2 | 1.0 | 0.0 | 0.4 | 0.0 |
| 38F | GTF1645/mut3325 | 0.4 | 97.2 | 1.2 | 0.2 | 0.6 | 0.2 | 0.2 | 0.0 |
| 38G | GTF6099/mut3325 | 0.4 | 97.4 | 1.1 | 0.2 | 0.6 | 0.2 | 0.2 | 0.0 |
| 38H | GTF7317/mut3325 | 0.6 | 97.0 | 1.6 | 0.2 | 0.1 | 0.0 | 0.6 | 0.0 |
| 38I | GTF8487/mut3325 | 0.4 | 97.2 | 1.0 | 0.2 | 0.9 | 0.0 | 0.4 | 0.0 |
| 38J | GTF3879/mut3325 | 1.0 | 93.8 | 1.8 | 0.3 | 1.4 | 0.5 | 1.2 | 0.0 |
| 38K | GTF3808/mut3325 | 0.9 | 93.9 | 2.2 | 0.3 | 1.4 | 0.4 | 0.8 | 0.0 |
| 38L | GTF8467/mut3325 | 1.1 | 94.3 | 1.6 | 0.3 | 1.5 | 0.4 | 0.8 | 0.0 |
| 38M | GTF0060/mut3325 | 1.0 | 92.7 | 2.2 | 1.3 | 1.3 | 0.4 | 1.1 | 0.0 |
| 38N | GTF0479/mut3325 | 1.0 | 93.9 | 2.1 | 0.3 | 1.3 | 0.4 | 1.1 | 0.0 |
| 38O | GTF0487/mut3325 | 1.9 | 30.0 | 3.2 | 1.0 | 61.5 | 0.3 | 0.2 | 1.8 |
| 38P | GTF5360/mut3325 | 1.0 | 33.0 | 1.9 | 0.4 | 63.6 | 0.0 | 0.2 | 0.0 |

### Example 39B

### Comparison of Anomeric Linkage Analysis of Soluble Oligosaccharide Fiber Produced by GTF-S Homologs and C-Terminal Truncated GTF-S Homologs in Combination with MUT3325

Solutions of chromatographically-purified soluble oligosaccharide fibers prepared as described in Examples 38Q to Example 38S were dried to a constant weight by lyophilization, and the resulting solids analyzed by ¹H NMR spectroscopy and by GC/MS as described in the General Methods section (above). The anomeric linkages for each of these soluble oligosaccharide fiber mixtures are reported in Tables 54 and 55, and compared to their respective non-truncated homologs.

TABLE 54
Anomeric linkage analysis of soluble oligosaccharides
by ¹H NMR spectroscopy.

| | | % | % | % | % |
|---|---|---|---|---|---|
| Example | | α- | α- | α- | α- |
| # | GTF/mutanase | (1, 3) | (1, 3, 6) | (1, 2, 6) | (1, 6) |
| 38A | GTF0974/mut3325 | 94.0 | 1.0 | 0.0 | 5.0 |
| 38Q | GTF0974-T4/mut3325 | 94.8 | 0.0 | 0.0 | 5.2 |
| 38R | GTF0974-T5/mut3325 | 94.7 | 0.0 | 0.0 | 5.3 |
| 38K | GTF3808/mut3325 | 93.4 | 0.0 | 0.0 | 6.6 |
| 38S | GTF3808-T5/mut3325 | 94.7 | 0.0 | 0.0 | 5.3 |

TABLE 55
Anomeric linkage analysis of soluble oligosaccharides by GC/MS.

| | | | | | | | % | % | % |
|---|---|---|---|---|---|---|---|---|---|
| | | % | % | % | % | % | α- | α- | α-(1,4,6) + α- |
| Example # | GTF/mutanase | α-(1,4) | α-(1,3) | α-(1,3,6) | α-(1,2) | α-(1,6) | (1,3,4) | (1,2,3) | (1,2,6) |
| 38A | GTF0974/mut3325 | 0.6 | 96.0 | 1.5 | 0.2 | 1.1 | 0.2 | 0.4 | 0.0 |
| 38Q | GTF0974-T4/mut3325 | 0.5 | 96.3 | 1.3 | 0.1 | 0.9 | 0.4 | 0.5 | 0.0 |
| 38R | GTF0974-T5/mut3325 | 0.5 | 96.5 | 1.4 | 0.1 | 0.9 | 0.2 | 0.4 | 0.0 |
| 38K | GTF3808/mut3325 | 0.9 | 93.9 | 2.2 | 0.3 | 1.4 | 0.4 | 0.8 | 0.0 |
| 38S | GTF3808-T5/mut3325 | 0.5 | 96.2 | 1.3 | 0.2 | 1.1 | 0.2 | 0.4 | 0.0 |

### Example 40

### Viscosity of Soluble Oligosaccharide Fiber Produced by GTF-J and by GTF/Mutanase Combinations

Solutions of chromatographically-purified soluble oligosaccharide fibers prepared as described in Examples 19 to Example 26 were dried to a constant weight by lyophilization, and the resulting solids were used to prepare a 12 wt % solution of soluble fiber in distilled, deionized water. The viscosity of the soluble fiber solutions (reported in centipoise (cP), where 1 cP=1 millipascal-s (mPa-s)) (Table 56) was measured at 20° C. as described in the General Methods section.

TABLE 56
Viscosity of 12% (w/w) soluble oligosaccharide fiber solutions
measured at 20° C. (ND = not determined).

| Example | | viscosity |
|---|---|---|
| # | GTF/mutanase | (cP) |
| 31 | GTF7527/mut3264 | 1.4 |
| 32 | GTF2379/mut3264 | ND |
| 33 | GTF7527/mut3325 | 2.0 |
| 34 | GTF0874/mut3325 | 1.6 |
| 35 | GTF0459/mut3264 | 1.7 |
| 36 | GTF0544/mut3264 | 6.7 |
| 37 | GTF0459/mut3325 | 1.8 |
| 38 | GTF7527/no mutanase | ND |

### Example 40A

### Viscosity of Soluble Oligosaccharide Fiber Produced by GTF-S, GTF-S Homologs and GTF-S Non-Homologs in Combination with MUT3325

Solutions of chromatographically-purified soluble oligosaccharide fibers prepared as described in Examples 38A to Example 38P were dried to a constant weight by lyophilization, and the resulting solids were used to prepare a 12 wt % solution of soluble fiber in distilled, deionized water. The viscosity of the soluble fiber solutions (reported in centipoise (cP), where 1 cP=1 millipascal-s (mPa-s)) (Table 57) was measured at 20° C. as described in the General Methods section.

TABLE 57
Viscosity of 12% (w/w) soluble oligosaccharide
fiber solutions measured at 20° C.

| Example | | viscosity |
|---|---|---|
| # | GTF/mutanase | (cP) |
| 38A | GTF0974/mut3325 | 1.8 |
| 38B | GTF4336/mut3325 | 1.7 |
| 38C | GTF0470/mut3325 | 1.7 |
| 38D | GTF6549/mut3325 | 1.7 |
| 38E | GTF4491/mut3325 | 1.7 |
| 38F | GTF1645/mut3325 | 1.6 |
| 38G | GTF6099/mut3325 | 1.6 |
| 38H | GTF7317/mut3325 | 1.6 |
| 38I | GTF8487/mut3325 | 1.7 |
| 38J | GTF3879/mut3325 | 1.6 |
| 38K | GTF3808/mut3325 | 4.1 |
| 38L | GTF8467/mut3325 | 4.0 |
| 38M | GTF0060/mut3325 | 4.0 |
| 38N | GTF0479/mut3325 | 4.2 |
| 38O | GTF0487/mut3325 | 2.1 |
| 38P | GTF5360/mut3325 | 1.9 |
| 38Q | GTF0974T4/mut3325 | 1.7 |
| 38R | GTF0974T5/mut3325 | 1.7 |
| 38S | GTF3808T5/mut3325 | 1.7 |

### Example 41

### Digestibility of Soluble Oligosaccharide Fiber Produced by GTF-J and by GTF/Mutanase Combinations

Solutions of chromatographically-purified soluble oligosaccharide fibers prepared as described in Examples 19 to Example 26 and Examples 38A to Example 38P were dried to a constant weight by lyophilization. The digestibility test protocol was adapted from the Megazyme Integrated Total Dietary Fiber Assay (AOAC method 2009.01, Ireland). The final enzyme concentrations were kept the same as the AOAC method: 50 Unit/mL of pancreatic α-amylase (PAA), 3.4 Units/mL for amyloglucosidase (AMG). The substrate concentration in each reaction was 25 mg/mL as recommended by the AOAC method. The total volume for each reaction was 1 mL instead of 40 mL as suggested by the original protocol. Every sample was analyzed in duplicate with and without the treatment of the two digestive enzymes. The detailed procedure is described below:

The enzyme stock solution was prepared by dissolving 20 mg of purified porcine pancreatic α-amylase (150,000 Units/g; AOAC Method 2002.01) from the Integrated Total Dietary Fiber Assay Kit in 29 mL of sodium maleate buffer (50 mM, pH 6.0 plus 2 mM CaCl2) and stir for 5 min, followed by the addition of 60 uL amyloglucosidase solution (AMG, 3300 Units/mL) from the same kit. 0.5 mL of the enzyme stock solution was then mixed with 0.5 mL soluble fiber sample (50 mg/mL) in a glass vial and the digestion reaction mixture was incubated at 37° C. and 150 rpm in orbital motion in a shaking incubator for exactly 16 h. Duplicated reactions were performed in parallel for each fiber sample. The control reactions were performed in duplicate by mixing 0.5 mL maleate buffer (50 mM, pH 6.0 plus 2 mM CaCl2) and 0.5 mL soluble fiber sample (50 mg/mL) and reaction mixtures was incubated at 37° C. and 150 rpm in orbital motion in a shaking incubator for exactly 16 h. After 16 h, all samples were removed from the incubator and immediately 75 μL of 0.75 M TRIZMA® base solution was added to terminate the reaction. The vials were immediately placed in a heating block at 95-100° C., and incubate for 20 min with occasional shaking (by hand). The total volume of each reaction mixture is 1.075 mL after quenching. The amount of released glucose in each reaction was quantified by HPLC with the Aminex HPX-87C Columns (BioRad) as described in the General Methods. Maltodextrin (DE4-7, Sigma) was used as the positive control for the enzymes (Tables 58-60).

To calculate the digestibility, the following formula was used:

Digestibility=100%*[amount of glucose (mg) released after treatment with enzyme−amount of glucose (mg) released in the absence of enzyme]/1.1*amount of total fiber (mg)

TABLE 58
Digestibility of soluble oligosaccharide fiber.

| Example | | Digestibility |
|---|---|---|
| # | GTF/mutanase | (%) |
| 31 | GTF7527/mut3264 | 7.0 |
| 32 | GTF2379/mut3264 | ND |
| 33 | GTF7527/mut3325 | 0.0 |
| 34 | GTF0874/mut3325 | 8.2 |
| 35 | GTF0459/mut3264 | 0.0 |
| 36 | GTF0544/mut3264 | 9.0 |
| 37 | GTF0459/mut3325 | 2.1 |
| 38 | GTF7527/no mutanase | 0.0 |

TABLE 59
Digestibility of soluble oligosaccharide fiber.

| Example | | Digestibility |
|---|---|---|
| # | GTF/mutanase | (%) |
| 38A | GTF0974/mut3325 | 2.1 |
| 38B | GTF4336/mut3325 | 2.2 |
| 38C | GTF0470/mut3325 | 1.7 |
| 38D | GTF6549/mut3325 | 2.1 |
| 38E | GTF4491/mut3325 | 2.2 |
| 38F | GTF1645/mut3325 | 2.3 |
| 38G | GTF6099/mut3325 | 1.6 |
| 38H | GTF7317/mut3325 | 2.2 |
| 38I | GTF8487/mut3325 | 2.0 |
| 38J | GTF3879/mut3325 | 0.74 |
| 38K | GTF3808/mut3325 | 2.1 |
| 38L | GTF8467/mut3325 | 0.28 |
| 38M | GTF0060/mut3325 | 1.0 |
| 38N | GTF0479/mut3325 | 1.4 |
| 38O | GTF0487/mut3325 | 5.9 |
| 38P | GTF5360/mut3325 | 9.4 |

TABLE 60
Digestibility of soluble oligosaccharide fiber.

| Example | | Digestibility |
|---|---|---|
| # | GTF/mutanase | (%) |
| 38Q | GTF0974-T4/mut3325 | 0.59 |
| 38R | GTF0974-T5/mut3325 | 0.44 |
| 38S | GTF3808-T5/mut3325 | 1.00 |

### Example 42

### In Vitro Gas Production using Soluble Oligosaccharide/Polysaccharide Fiber as Carbon Source

Solutions of chromatographically-purified soluble oligosaccharide/polysaccharide fibers were dried to a constant weight by lyophilization. The individual soluble oligosaccharide/polysaccharide soluble fiber samples were subsequently evaluated as carbon source for in vitro gas production using the method described in the General Methods. PROMITOR® 85 (soluble corn fiber, Tate & Lyle), NUTRIOSE® FM06 (soluble corn fiber or dextrin, Roquette), FIBERSOL-2® 600F (digestion-resistant maltodextrin, Archer Daniels Midland Company & Matsutani Chemical), ORAFTI® GR (inulin from Beneo, Mannheim, Germany), LITESSE® Ultra™ (polydextrose, Danisco), GOS (galactooligosaccharide, Clasado Inc., Reading, UK), ORAFTI® P95 (oligofructose (fructo-oligosaccharide, FOS, Beneo), LACTITOL MC (4-O-β-D-Galactopyranosyl-D-glucitol monohydrate, Danisco) and glucose were included as control carbon sources. Table 61 lists the In vitro gas production by intestinal microbiota at 3 h and 24 h. Table 62 lists the in vitro gas production by intestinal microbiota from controls and the sequences identified from the GTF0459 homolog sequence search. Table 63 lists the in vitro gas production by intestinal microbiota from controls and truncations of homolog sequences identified from the GTF0459 homolog sequence search.

TABLE 61
In vitro gas production by intestinal microbiota.

| | mL gas | mL gas |
|---|---|---|
| | formation | formation |
| Sample | in 3 h | in 24 h |
| PROMITOR ® 85 | 2.6 | 8.5 |
| NUTRIOSE ® FM06 | 3.0 | 9.0 |
| FIBERSOL-2 ® 600F | 2.8 | 8.8 |
| ORAFTI ® GR | 3.0 | 7.3 |
| LITESSE ® ULTRA ™ | 2.3 | 5.8 |
| GOS | 2.6 | 5.2 |
| ORAFTI ® P95 | 2.6 | 7.5 |
| LACTITOL ® MC | 2.0 | 4.8 |
| Glucose | 2.4 | 5.2 |
| GTF7527 47° C. | 4.0 | 7.8 |
| GTF7527/mut3325 | 3.7 | 6.7 |
| GTF0459/mut3264 | 3.7 | 6.7 |
| GTF0459/mut3325 | 3.5 | 5.5 |
| GTF0874/mut3325 | 4.0 | 7.0 |

TABLE 62
In vitro gas production by intestinal microbiota..

| | | mL gas | mL gas | mL gas |
|---|---|---|---|---|
| Example | | formation | formation | formation |
| # | GTF/mutanase | in 3 h | in 26 h | in 24.5 h |
| | ORAFTI ® GR | 4.0 | | 8.0 |
| | LITESSE ® ULTRA ™ | 2.0 | | 6.0 |
| | LACTITOL ® MC | 2.0 | | 1.5 |
| | Glucose | 2.0 | | 1.5 |
| 38A | GTF0974/mut3325 | 3.5 | 2.0 | |
| 38B | GTF4336/mut3325 | 3.0 | 2.0 | |
| 38C | GTF0470/mut3325 | 3.5 | | 2.5 |
| 38D | GTF6549/mut3325 | 4.0 | | 2.0 |
| 38E | GTF4491/mut3325 | 4.0 | | 2.0 |
| 38F | GTF1645/mut3325 | 4.0 | | 2.0 |
| 38G | GTF6099/mut3325 | 3.5 | | 2.0 |
| 38H | GTF7317/mut3325 | 3.5 | | 2.0 |
| 38I | GTF8487/mut3325 | 3.5 | | 2.0 |
| 38J | GTF3879/mut3325 | 3.0 | | 2.0 |
| 38K | GTF3808/mut3325 | 3.0 | | 2.0 |
| 38L | GTF8467/mut3325 | 2.5 | | 2.0 |
| 38M | GTF0060/mut3325 | 3.0 | | 2.0 |
| 38N | GTF0479/mut3325 | 2.5 | | 2.0 |
| 38O | GTF0487/mut3325 | 3.5 | | 2.5 |
| 38P | GTF5360/mut3325 | 3.5 | | 3.0 |

TABLE 63
In vitro gas production by intestinal microbiota..

| | | mL gas | mL gas |
|---|---|---|---|
| Example | | formation | formation |
| # | GTF/mutanase | in 3 h | in 24.5 h |
| | LITESSE ® ULTRA ™ | 3.5 | 7.0 |
| | LACTITOL ® MC | 3.0 | 2.0 |
| | Glucose | 3.5 | 2.0 |
| 38Q | GTF0974-T4/mut3325 | 4.0 | 2.0 |
| 38R | GTF0974-T5/mut3325 | 4.0 | 2.0 |
| 38S | GTF3808-T5/mut3325 | 4.0 | 2.0 |

### Example 43

### Colonic Fermentation Modeling and Measurement of Fatty Acids

Colonic fermentation was modeled using a semi-continuous colon simulator as described by Makivuokko et al. (Nutri. Cancer (2005) 52(1):94-104); in short; a colon simulator consists of four glass vessels which contain a simulated ileal fluid as described by Macfarlane et al. (Microb. Ecol. (1998) 35(2):180-187). The simulator is inoculated with a fresh human faecal microbiota and fed every third hour with new ileal liquid and part of the contents is transferred from one vessel to the next. The ileal fluid contains one of the described test components at a concentration of 1%. The simulation lasts for 48 h after which the content of the four vessels is harvested for further analysis. The further analysis involves the determination of microbial metabolites such as short chain fatty acids (SCFA); also referred to as volatile fatty acids (VFA) and branched chain fatty acids (BCFA). Analysis was performed as described by Holben et al. (Microb. Ecol. (2002) 44:175-185); in short; simulator content was centrifuged and the supernatant was used for SCFA and BCFA analysis. Pivalic acid (internal standard) and water were mixed with the supernatant and centrifuged. After centrifugation, oxalic acid solution was added to the supernatant and then the mixture was incubated at 4° C., and then centrifuged again. The resulting supernatant was analyzed by gas chromatography using a flame ionization detector and helium as the carrier gas. Comparative data generated from samples of LITESSE® ULTRA™ (polydextrose, Danisco), ORAFTI® P95 (oligofructose; fructo-oligosaccharide, “FOS”, Beneo), lactitol (Lactitol MC (4-O-β-D-galactopyranosyl-D-glucitol monohydrate, Danisco), and a negative control is also provided. The concentration of acetic, propionic, butyric, isobutyric, valeric, isovaleric, 2-methylbutyric, and lactic acid was determined (Table 64).

TABLE 64
Simulator metabolism and measurement of fatty acid production.

| | | | | | | Short | Branched |
|---|---|---|---|---|---|---|---|
| | | | | | | Chain | Chain |
| | | | | | | Fatty | Fatty |
| | | | | | | Acids | Acids |
| | Acetic | Propionic | Butyric | Lactic | Valeric | (SCFA) | (BCFA) |
| Sample | (mM) | (mM) | (mM) | (mM) | (mM) | (mM) | (mM) |
| GTF7527/MUT | 340 | 55 | 233 | 1 | 6 | 585 | 4.9 |
| 3325 | | | | | | | |
| GTF0459/MUT | 407 | 55 | 200 | 10 | 5 | 678 | 4.7 |
| 3264 | | | | | | | |
| GTF7527- | 103 | 6.5 | 9.0 | 114 | 2 | 235 | 1.0 |
| 47° C. | | | | | | | |
| GTF0459/MUT | 442 | 73 | 169 | 18 | 2 | 704 | 3.6 |
| 3325 | | | | | | | |
| Control | 83 | 31 | 40 | 3 | 6 | 163 | 7.2 |
| LITESSE ® | 256 | 76 | 84 | 1 | 6 | 423 | 5.3 |
| polydextrose | | | | | | | |
| FOS | 91 | 9 | 8 | 14 | — | 152 | 2.1 |
| Lactitol | 318 | 42 | 94 | 52 | — | 506 | 7.5 |

### Example 44

### Preparation of Extracts of Glucosyltransferase (GTF) Enzymes for Fiber Production at Different Temperatures

The Streptococcus salivarius gtfJ enzyme (SEQ ID NO: 5) used in Examples 1 and 2 was expressed in E. coli strain DH10B using an isopropyl beta-D-1-thiogalactopyranoside (IPTG)-induced expression system. Briefly, E. coli DH10B cells were transformed to express SEQ ID NO: 5 from a DNA sequence (SEQ ID NO:4) codon-optimized to express the gtfJ enzyme in E. coli. This DNA sequence was contained in the expression vector, PJEXPRESS404® (DNA 2.0, Menlo Park Calif.). The transformed cells were inoculated to an initial optical density (OD at 600ₙₘ) of 0.025 in LB medium (10 g/L Tryptone; 5 g/L yeast extract, 10 g/L NaCl) and allowed to grow at 37° C. in an incubator while shaking at 250 rpm. The cultures were induced by addition of 1 mM IPTG when they reached an OD₆₀₀ of 0.8-1.0. Induced cultures were left on the shaker and harvested 3 hours post induction.

For harvesting gtfJ enzyme (SEQ ID NO: 5), the cells were centrifuged (25° C., 16,000 rpm) in an EPPENDORF® centrifuge, re-suspended in 5.0 mM phosphate buffer (pH 7.0) and cooled to 4° C. on ice. The cells were broken using a bead beater with 0.1 mm silica beads, and then centrifuged at 16,000 rpm at 4° C. to pellet the unbroken cells and cell debris. The crude extract (containing soluble gtfJ enzyme, SEQ ID NO: 5) was separated from the pellet and analyzed by Bradford protein assay to determine protein concentration (mg/mL).

The additional gtf enzymes used in Example 45 were prepared as follows. E. coli TOP10® cells (Invitrogen, Carlsbad Calif.) were transformed with a PJEXPRESS404®-based construct containing a particular gtf-encoding DNA sequence. Each sequence was codon-optimized to express the gtf enzyme in E. coli. Individual E. coli strains expressing a particular gtf enzyme were grown in LB medium with ampicillin (100 mg/mL) at 37° C. with shaking to OD₆₀₀=0.4-0.5, at which time IPTG was added to a final concentration of 0.5 mM. The cultures were incubated for 2-4 hours at 37° C. following IPTG induction. Cells were harvested by centrifugation at 5,000×g for 15 minutes and resuspended (20% w/v) in 50 mM phosphate buffer pH 7.0 supplemented with DTT (1.0 mM). Resuspended cells were passed through a French Pressure Cell (SLM Instruments, Rochester, N.Y.) twice to ensure >95% cell lysis. Lysed cells were centrifuged for 30 minutes at 12,000×g at 4° C. The resulting supernatant was analyzed by the BCA protein assay and SDS-PAGE to confirm expression of the gtf enzyme, and the supernatant was stored at −20° C.

### Analysis of Reaction Profiles

Periodic samples from reaction mixtures were taken and analyzed using an Agilent 1260C HPLC equipped with a refractive index detector. An Aminex HP-87C column, (BioRad) using deionized water at a flow rate of 0.6 mL/min and 85° C. was used to monitor sucrose and glucose. An Aminex HP-42A column (BioRad) using deionized water at a flow rate of 0.6 mL/min and 85° C. was used to quantitate oligosaccharides from DP2-DP7 which were previously calibrated using malto oligosaccharides.

### Example 45

### Oligosaccharide Production using GTF-J at Various Temperatures

The desired amount of sucrose, in some cases glucose, and 20 mM dihydrogen potassium phosphate were dissolved using deionized water and diluted to 750 mL in a 1 L unbaffled jacketed flask that was connected to a Lauda RK20 recirculating chiller. FERMASURE™ (DuPont, Wilmington, Del.) was then added (0.5 mL/L reaction), and the pH was adjusted to 5.5 using 5 wt % aqueous sodium hydroxide or 5 wt % aqueous sulfuric acid. The reaction was initiated by the addition of 0.3 vol % of crude enzyme extract containing GTF-J (SEQ ID NO: 5) as described in Example 44. Agitation to the reaction mixture was provided using a 4-blade PTFE overhead mechanical mixer at 100 rpm. After the reaction was determined to be complete by either complete consumption of sucrose or no change in sucrose concentration between subsequent measurements, the reaction slurry was filtered to remove the insoluble polymer. Yields of the soluble oligosaccharides were determined by HPLC according to the method in Example 44 and are presented in Table 65.

TABLE 65
Yield of oligosaccharides using gtf-J
under various operating conditions.

| | | | | g oligomers/ | g leucrose/ |
|---|---|---|---|---|---|
| T | Glucose | Sucrose | % sucrose | g sucrose | g sucrose |
| (° C.) | (g/L, t = 0) | (g/L, t = 0) | converted | reacted | reacted |
| 25 | 0 | 94.9 | 95 | 0.12 | 0.32 |
| 25 | 25.2 | 100.4 | 93 | 0.30 | 0.21 |
| 25 | 0 | 407.9 | 96 | 0.20 | 0.56 |
| 42 | 0 | 94.5 | 99 | 0.13 | 0.26 |
| 47 | 0 | 95.0 | 90 | 0.25 | 0.35 |
| 47 | 25.7 | 101.1 | 92 | 0.39 | 0.15 |
| 47 | 103.4 | 102.1 | 81 | 0.65 | 0.09 |
| 47 | 26.6 | 255.7 | 94 | 0.26 | 0.23 |
| 47 | 105.2 | 408.4 | 91 | 0.47 | 0.26 |
| 47 | 27.6 | 415.3 | 94 | 0.29 | 0.33 |

These results demonstrate that the yield of soluble oligosaccharides is increased when the reaction is run above 42° C., that the yield of oligosaccharides can be further increased by adding an acceptor molecule, such as glucose, and that the amount of leucrose formed decreases upon addition of an acceptor molecule.

### Example 46

### Oligosaccharide Production using Other GTF Enzymes

The desired amount of sucrose and 20 mM dihydrogen potassium phosphate were dissolved using deionized water and transferred to a glass bottle equipped with a polypropylene cap. Fermasure™ (DuPont, Wilmington, Del.) was then added (0.5 mL/L reaction), and the pH was adjusted to 5.5 using 5 wt % aqueous sodium hydroxide or 5 wt % aqueous sulfuric acid. The reaction was initiated by the addition of crude enzyme extract as prepared in Example 44. Additional truncated GTFs from the following were tested: Streptococcus sobrinus (GTF0874; SEQ ID NO: 16), Streptococcus downei (GTF1724; SEQ ID NO: 81), and Streptococcus dentirousetti (GTF5926; SEQ ID NO: 84). Agitation to the reaction mixture was provided using either a PTFE stirbar or an Inova 42 incubator shaker, and the reaction was heated either using a block heater or the incubator shaker. After the reaction was determined to be complete by either complete consumption of sucrose or no change in sucrose concentration between subsequent measurements, the reaction slurry was filtered to remove the insoluble polymer. Yield of the soluble oligosaccharides was determined by HPLC according to the method in Example 44 and are presented in Table 66.

TABLE 66
Comparison of oligomer yield using gtf enzymes under various
operating conditions.

| | SEQ | | Sucrose | | g oligomer / | g leucrose / |
|---|---|---|---|---|---|---|
| Scale | ID | T | (g/L, | % sucrose | g sucrose | g sucrose |
| (mL) | NO | (° C.) | t = 0) | converted | reacted | reacted |
| 100 | 16 | 37 | 146.0 | 97 | 0.24 | 0.39 |
| 10 | 16 | 50 | 149.1 | 95 | 0.30 | 0.24 |
| 100 | 81 | 37 | 146.1 | 99 | 0.25 | 0.33 |
| 10 | 81 | 50 | 149.1 | 99 | 0.33 | 0.24 |
| 100 | 84 | 37 | 145.8 | 74 | 0.21 | 0.29 |
| 10 | 84 | 50 | 149.1 | 99 | 0.30 | 0.28 |

These results demonstrate that behavior described in Example 44 is general to other gtf enzymes.

### Example 47

### Preparation of a Fiber Composition Containing the Soluble α-Glucan Fiber

This example describes the preparation of a composition containing the soluble α-glucan fiber disclosed herein.

Two soluble α-glucan fiber compositions were produced according to the processes disclosed above. The Brix and the concentration of oligosaccharides was determined by HPLC according to the previously given procedure. The results are shown in Table 67. The composition was used to produce fiber water, spoonable yogurt, and a snack bar, as described below.

TABLE 67
Properties of the soluble α-glucan fiber compositions.

| | soluble α- | soluble α- |
|---|---|---|
| DP | glucan fiber 1 | glucan fiber 2 |
| DP7+ | 14.5 | 48.3 |
| DP6 | 20.6 | 15.9 |
| DP5 | 24.3 | 14.2 |
| DP4 | 21.7 | 9.9 |
| DP3 | 10.6 | 5.0 |
| DP2 | 2.3 | 3.0 |
| Brix | 71.1 | 52.0 |

### Example 48

### Preparation of Fiber Water Formulations

The following example describes the preparation of fiber water formulations using the fiber compositions produced in Example 47.

TABLE 68
Components of the fiber water formulations

| | Fiber Water | Fiber Water |
|---|---|---|
| | Formulation 1 | Formulation 2 |
| Ingredient | Ingredient (grams) | |
| Water, deionized | 12852.02 | 12325.52 |
| Antho-Red 03899, food coloring | 1.13 | 1.13 |
| (available from Sensient | | |
| Technologies Corporation, | | |
| Milwaukee, Wisconsin) | | |
| Soluble α-glucan Fiber 1 | 1290.0 | 0 |
| Soluble α-glucan Fiber 2 | 0 | 1816.5 |
| Sucrose (available from Domino | 784.5 | 784.5 |
| Sugar, Baltimore, Maryland) | | |
| Citric Acid, anhydrous | 15.0 | 15.0 |
| (available from Jungbunzlauer | | |
| Jungbunzlauer Suisse AG, Basel, | | |
| Switzerland) | | |
| Cherry Flavor, available from | 1.50 | 1.50 |
| Virginia Dare, Brooklyn, New | | |
| York) | | |
| Raspberry Flavor, available from | 30.0 | 30.0 |
| Virginia Dare | | |
| Cranberry Flavor, available from | 19.95 | 19.95 |
| Virginia Dare | | |
| Salt (available from Cargill, | 2.25 | 2.25 |
| Minneapolis, Minnesota) | | |
| Vitamin C, ascorbic acid | 3.66 | 3.66 |

Two fiber water formulations were produced using the fiber composition of example 47. Deionized water was added to a suitable mixing vessel. The soluble α-glucan fiber, sucrose, citric acid, ascorbic acid and salt were added to the mixing vessel and the resulting mixture was blended for 5 minutes. The components of the mixture were added in the amounts detailed in table 68. Following the blending step, the red food coloring, the cherry flavor, raspberry flavor and cranberry flavors were added to the water mixture, with stirring. After this addition was completed, the mixture was subjected to an ultra-high temperature (UHT) pasteurization for 7 seconds at 106.7° C. at 3000 pounds per square inch (psig) (20.7 MPa) and the mixture was homogenized at 2500/500 psig (17.24/3.45 MPa) using an indirect steam (IDS) unit. The mixture was added to bottles and the bottles were cooled in an ice bath before storing in a refrigerator.

### Example 49

### Preparation of a Spoonable Yogurt Formulation

The following example describes the preparation of two spoonable yogurts using the fiber compositions produced in Example 47.

TABLE 69
Components of spoonable yogurts

| | Yogurt 1 | Yogurt 2 |
|---|---|---|
| Ingredient | Ingredient (grams) | |
| Skim Milk | 2986.84 | 2813.73 |
| Whole Milk | 687.46 | 686.47 |
| THERMTEX ® modified food | 121.5 | 121.5 |
| starch (available from Ingredion, | | |
| Bridgewater, New Jersey) | | |
| gelatin (250 B) | 13.5 | 13.5 |
| Nonfat diary milk solids | 78.70 | 94.85 |
| Sucrose | 225.0 | 225.0 |
| YO-MIX ® 860 yogurt Cultures | 20 DCU/100 L | 20 DCU/100 L |
| (add to pH break point), available | | |
| from DuPont Danisco, | | |
| Wilmington, Delaware | | |
| Soluble α-glucan fiber 1 | 387.0 | 0 |
| Soluble α-glucan fiber 2 | 0 | 544.95 |
| TOTAL | 4500.0 | 4500.0 |

Two spoonable yogurts were made using the ingredients detailed in table 69. The THERMTEX® food starch, gelatin (250 B) and the nonfat dairy milk solids were blended. This blend of solids was then added to a mixture of the whole and skim milk. The soluble α-glucan fiber was also added to the milk and the mixture was stirred. This mixture was pasteurized at 87.2° C. for 30 minutes via vat pasteurization. The pasteurized mixture was then homogenized in a two-stage homogenizer at 17.24 MPa (first stage) and 3.45 MPa (second stage). The mixture was then cooled to 43.3° C. and was inoculated with the yogurt culture. The inoculated culture was incubated to a pH of 4.6. After incubation, the mixture was cooled to 4.4° C. in a yogurt press. After cooling, the yogurt was packaged and stored in a refrigerator.

### Example 50

### Preparation of a Snack Bar

The following example describes the preparation of a snack bar using the fiber compositions produced in Example 47.

TABLE 70
Components of the snack bar

| Ingredients | Grams |
|---|---|
| DU-CROSE 63/43, corn syrup | 787.44 |
| Soluble α-glucan fiber 1 | 865.98 |
| SUPRO ® nugget 309 soy protein | 1155.15 |
| nuggets (available from DuPont | |
| Danisco, Wilmington, Delaware) | |
| Rolled Oats | 972.57 |
| Vanilla Cream | 33.66 |
| Bake Shoppe mini baking chip, | 379.95 |
| chocolate (available from The | |
| Hershey Company, Inc, Hershey, | |
| Pennsylvania) | |
| Coconut oil | 54.57 |
| Arabic Gum | 124.44 |
| Russet Cocoa Powder, 10-12% fat | 51.51 |
| Milk Chocolate Coating Compound | 674.73 |

A snack bar was prepared from the components detailed in table 70. The corn syrup and the soluble α-glucan fiber 1 were added to a suitable mixing vessel and warmed to 37.8° C. In a separate vessel, the coconut oil was heated to melt the oil. The liquid coconut oil was added to the corn syrup/fiber mixture and stirred for one minute. The SUPRO® soy protein nuggets, rolled oats, vanilla cream, mini baking chips, arabic gum and the cocoa powder was added to the mixture and stirred for 30 seconds. After stirring, the solids were scraped off of the sides of the mixing vessel and the stirring was continued until a dough formed. The dough was formed into bars and the bars were coated with the milk chocolate coating compound.

### Example 51

### Preparation of a Yogurt-Drinkable Smoothie

The following example describes a method for the preparation of a yogurt-drinkable smoothie using the present fibers.

TABLE 71

| Ingredients | wt % |
|---|---|
| Distilled Water | 49.00 |
| Supra XT40 Soy Protein Isolate | 6.50 |
| Fructose | 1.00 |
| Grindsted ASD525, Danisco | 0.30 |
| Apple Juice Concentrate (70 Brix) | 14.79 |
| Strawberry Puree, Single Strength | 4.00 |
| Banana Puree, Single Strength | 6.00 |
| Plain Lowfat Yogurt - Greek Style, Cabot | 9.00 |
| 1% Red 40 Soln | 0.17 |
| Strawberry Flavor (DD-148-459-6) | 0.65 |
| Banana Flavor (#29513) | 0.20 |
| 75/25 Malic/Citric Blend | 0.40 |
| Present Soluble Fiber Sample | 8.00 |
| Total | 100.00 |

Step No. Procedure

Pectin Solution Formation
1 Heat 50% of the formula water to 160° F. (˜71.1° C.).
2 Disperse the pectin with high shear; mix for 10 minutes.
3 Add the juice concentrates and yogurt; mix for 5-10 minutes until the yogurt is dispersed.

Protein Slurry
1 Into 50% of the batch water at 140° F. (60° C.), add the Supro XT40 and mix well.
2 Heat to 170° F. (˜76.7° C.) and hold for 15 minutes.
3 Add the pectin/juice/yogurt slurry to the protein solution; mix for 5 minutes.
4 Add the fructose, fiber, flavors and colors; mix for 3 minutes.
5 Adjust the pH using phosphoric acid to the desired range (pH range 4.0-4.1).
6 Ultra High Temperature (UHT) process at 224° F. (˜106.7° C.) for 7 seconds with UHT homogenization after heating at 2500/500 psig (17.24/3.45 MPa) using the indirect steam (IDS) unit.
7 Collect bottles and cool in ice bath.
8 Store product in refrigerated conditions.

### Example 52

### Preparation of a High Fiber Wafer

The following example describes the preparation of a high fiber wafer with the present fibers.

TABLE 72

| Ingredients | wt % |
|---|---|
| Flour, white plain | 38.17 |
| Present fiber | 2.67 |
| Oil, vegetable | 0.84 |
| GRINSTED ® CITREM 2-in-1¹ | 0.61 |
| citric acid ester made from sunflower | |
| or palm oil (emulsifier) | |
| Salt | 0.27 |
| Sodium bicarbonate | 0.11 |
| Water | 57.33 |

¹Danisco.

Step No. Procedure
1. High shear the water, oil and CITREM for 20 seconds.
2. Add dry ingredients slowly, high shear for 2-4 minutes.
3. Rest batter for 60 minutes.
4. Deposit batter onto hot plate set at 200° C. top and bottom, bake for 1 minute 30 seconds
5. Allow cooling pack as soon as possible.

### Example 53

### Preparation of a Soft Chocolate Chip Cookie

The following example describes the preparation of a soft chocolate chip cookie with the present fibers.

TABLE 73

| Ingredients | wt % |
|---|---|
| Stage 1 | |
| Lactitol, C | 16.00 |
| Cake margarine | 17.70 |
| Salt | 0.30 |
| Baking powder | 0.80 |
| Eggs, dried whole | 0.80 |
| Bicarbonate of soda | 0.20 |
| Vanilla flavor | 0.26 |
| Caramel flavor | 0.03 |
| Sucralose powder | 0.01 |
| Stage 2 | |
| Present Fiber Solution (70 brix) | 9.50 |
| water | 4.30 |
| Stage 3 | |
| Flour, pastry | 21.30 |
| Flour, high ratio cake | 13.70 |
| Stage Four | |
| Chocolate chips, 100% lactitol, | 15.10 |
| sugar free | |

Step No. Procedure

1. Cream together stage one, fast speed for 1 minute.

2. Blend stage two to above, slow speed for 2 minutes.

3. Add stage three, slow speed for 20 seconds.

4. Scrape down bowl; add stage four, slow speed for 20 seconds.

5. Divide into 30 g pieces, flatten, and place onto silicone lined baking trays.

6. Bake at 190° C. for 10 minutes approximately.

### Example 54

### Preparation of a Reduced Fat Short-Crust Pastry

The following example describes the preparation of a reduced fat short-crust pastry with the present fibers.

TABLE 74

| Ingredients | wt % |
|---|---|
| Flour, plain white | 56.6 |
| Water | 15.1 |
| Margarine | 11.0 |
| Shortening | 11.0 |
| Present fiber | 6.0 |
| Salt | 0.3 |

Step No. Procedure

1. Dry blend the flour, salt and present glucan fiber (dry)

2. Gently rub in the fat until the mixture resembles fine breadcrumbs.

3. Add enough water to make a smooth dough.

### Example 55

### Preparation of a Low Sugar Cereal Cluster

The following example describes the preparation of a low sugar cereal cluster with one of the present fibers.

TABLE 75

| Ingredients | wt % |
|---|---|
| Syrup Binder | 30.0 |
| Lactitol, MC 50% | |
| Present Fiber Solution (70 brix) | |
| 25% | |
| Water 25% | |
| Cereal Mix | 60.0 |
| Rolled Oats 70% | |
| Flaked Oats 10% | |
| Crisp Rice 10% | |
| Rolled Oats 10% | |
| Vegetable oil | 10.0 |

Step No. Procedure

1. Chop the fines.

2. Weight the cereal mix and add fines.

3. Add vegetable oil on the cereals and mix well.

4. Prepare the syrup by dissolving the ingredients.

5. Allow the syrup to cool down.

6. Add the desired amount of syrup to the cereal mix.

7. Blend well to ensure even coating of the cereals.

8. Spread onto a tray.

9. Place in a dryer/oven and allow to dry out.

10. Leave to cool down completely before breaking into clusters.

### Example 56

### Preparation of a Pectin Jelly

The following example describes the preparation of a pectin jelly with the present fibers.

TABLE 76

| Ingredients | wt % |
|---|---|
| Component A | |
| Xylitol | 4.4 |
| Pectin | 1.3 |
| Component B | |
| Water | 13.75 |
| Sodium citrate | 0.3 |
| Citric Acid, anhydrous | 0.3 |
| Component C | |
| Present Fiber Solution (70 brix) | 58.1 |
| Xylitol | 21.5 |
| Component D | |
| Citric acid | 0.35 |
| Flavor, Color | q.s. |

Step No. Procedure
1. Dry blend the pectin with the xylitol (Component A).
2. Heat Component B until solution starts to boil.
3. Add Component A gradually, and then boil until completely dissolved.
4. Add Component C gradually to avoid excessive cooling of the batch.
5. Boil to 113° C.
6. Allow to cool to <100° C. and then add colour, flavor and acid (Component D). Deposit immediately into starch molds.
7. Leave until firm, then de-starch.

### Example 57

### Preparation of a Chewy Candy

The following example describes the preparation of a chewy candy with the present fibers.

TABLE 77

| Ingredients | wt % |
|---|---|
| Present glucan fiber | 35 |
| Xylitol | 35 |
| Water | 10 |
| Vegetable fat | 4.0 |
| Glycerol Monostearate (GMS) | 0.5 |
| Lecithin | 0.5 |
| Gelatin 180 bloom (40% solution) | 4.0 |
| Xylitol, CM50 | 10.0 |
| Flavor, color & acid | q.s. |

Step No. Procedure
1. Mix the present glucan fiber, xylitol, water, fat, GMS and lecithin together and then cook gently to 158° C.
2. Cool the mass to below 90° C. and then add the gelatin solution, flavor, color and acid.
3. Cool further and then add the xylitol CM. Pull the mass immediately for 5 minutes.
4. Allow the mass to cool again before processing (cut and wrap or drop rolling).

### Example 58

### Preparation of a Coffee-Cherry Ice Cream

The following example describes the preparation of a coffee-cherry ice cream with the present fibers.

TABLE 78

| Ingredients | wt % |
|---|---|
| Fructose, C | 8.00 |
| Present glucan fiber | 10.00 |
| Skimmed milk powder | 9.40 |
| Anhydrous Milk Fat (AMF) | 4.00 |
| CREMODAN ® SE 709 | 0.65 |
| Emulsifier & Stabilizer System¹ | |
| Cherry Flavoring U35814¹ | 0.15 |
| Instant coffee | 0.50 |
| Tri-sodium citrate | 0.20 |
| Water | 67.10 |

¹Danisco.

Step No. Procedure

1. Add the dry ingredients to the water, while agitating vigorously.

2. Melt the fat.

3. Add the fat to the mix at 40° C.

4. Homogenize at 200 bar/70-75° C.

5. Pasteurize at 80-85° C./20-40 seconds.

6. Cool to ageing temperature (5° C.).

7. Age for minimum 4 hours.

8. Add flavor to the mix.

9. Freeze in continuous freezer to desired overrun (100% is recommended).

10. Harden and storage at −25° C.

What is claimed is:

1. A soluble α-glucan fiber composition comprising:
a. at least 75% α-(1,3) glycosidic linkages;
b. less than 25% α-(1,6) glycosidic linkages;
c. less than 10% α-(1,3,6) glycosidic linkages;
d. a weight average molecular weight of less than 5000 Daltons;
e. a viscosity of less than 0.25 Pascal second (Pa·s) at 12 wt % in water at 20° C.;
f. a dextrose equivalence (DE) in the range of 4 to 40; and
g. a digestibility of less than 12% as measured by the Association of Analytical Communities (AOAC) method 2009.01;
h. a solubility of at least 20% (w/w) in water at 25° C.; and
i. a polydispersity index of less than 5.

2. A carbohydrate composition comprising: 0.01 to 99 wt % (dry solids basis) of the soluble α-glucan fiber composition of claim 1.

3. A food product comprising the soluble α-glucan fiber composition of claim 1 or the carbohydrate composition of claim 2.

4. A method of producing a soluble α-glucan fiber composition comprising:
a. providing a set of reaction components comprising:
i. sucrose;
ii. at least one glucosyltransferase capable of catalyzing the synthesis of glucan polymers having at least 75% α-(1,3) glycosidic linkages;
iii. at least one α-glucanohydrolase capable of hydrolyzing glucan polymers having one or more α-(1,3) glycosidic linkages or one or more α-(1,6) glycosidic linkages; and
iv. optionally one or more acceptors;
b. combining the set of reaction components under suitable aqueous reaction conditions whereby a product comprising a soluble α-glucan fiber composition is produced; and
c. optionally isolating the soluble α-glucan fiber composition from the product of step (b).

5. A method to produce the soluble α-glucan fiber composition of claim 1 comprising:
a. providing a set of reaction components comprising:
i. sucrose;
ii. at least one glucosyltransferase capable of catalyzing the synthesis of glucan polymers having at least 75% α-(1,3) glycosidic linkages;
iii. at least one α-glucanohydrolase capable of hydrolyzing glucan polymers having one or more α-(1,3) glycosidic linkages or one or more α-(1,6) glycosidic linkages; and
iv. optionally one or more acceptors;
b. combining the set of reaction components under suitable aqueous reaction conditions to form a single reaction mixture, whereby a product mixture comprising glucose oligomers is formed;
c. isolating the soluble α-glucan fiber composition of claim 1 from the product mixture comprising glucose oligomers; and
d. optionally concentrating the soluble α-glucan fiber composition.

6. The method of claim 4 or 5 wherein combining the set of reaction components under suitable aqueous reaction conditions comprises combining the set of reaction components within a food product.

7. The method of claim 4 or 5 wherein the at least one glucosyltransferase comprises an amino acid sequence having at least 90% sequence identity to SEQ ID NO: 153.

8. The method of claim 4 or 5 wherein the at least one α-glucanohydrolase is a mutanase and the at least one glucosyltransferase and at least one mutanase comprise amino acid sequences having at least 90% identity to sequences selected from the following combinations of sequences, and truncations thereof:
a. glucosyltransferase GTF7527 (SEQ ID NOs: 3, 5 or a combination thereof) and mutanase MUT3325 (SEQ ID NO: 27);
b. glucosyltransferase GTF7527 (SEQ ID NOs: 3, 5 or a combination thereof) and mutanase MUT3264 (SEQ ID NOs: 21, 22, 24 or any combination thereof);
c. glucosyltransferase GTF0459 (SEQ ID NOs: 17, 19 or a combination thereof) or homologs thereof (SEQ ID NOs: 88, 90, 92, 94, 96, 98, 100, 102, 104, 106, 108, 110, 112 or a combination thereof) and mutanase MUT3325 (SEQ ID NO: 27); and
d. glucosyltransferase GTF0459 (SEQ ID NOs: 17, 19 or a combination thereof) or homologs thereof (SEQ ID NOs: 88, 90, 92, 94, 96, 98, 100, 102, 104, 106, 108, 110, 112 or a combination thereof) and mutanase MUT3264 (SEQ ID NOs: 21, 22, 24 or any combination thereof).

9. A method to produce the soluble α-glucan fiber composition of claim 1 comprising:
a. providing a set of reaction components comprising:
i. sucrose;
ii. at least one glucosyltransferase capable of catalyzing the synthesis of glucan polymers having one or more α-(1,3) glycosidic linkages; and
iii. optionally one or more acceptors;
b. combining the set of reaction components under suitable aqueous reaction conditions to form a single reaction mixture, wherein the reaction conditions comprise a reaction temperature greater than 45° C. and less than 55° C., whereby a product mixture comprising glucose oligomers is formed;
c. isolating the soluble α-glucan fiber composition of claim 1 from the product mixture comprising glucose oligomers; and
d. optionally concentrating the soluble α-glucan fiber composition.

10. A method to make a blended carbohydrate composition comprising combining the soluble α-glucan fiber composition of claim 1 with: a monosaccharide, a disaccharide, glucose, sucrose, fructose, leucrose, corn syrup, high fructose corn syrup, isomerized sugar, maltose, trehalose, panose, raffinose, cellobiose, isomaltose, honey, maple sugar, a fruit-derived sweetener, sorbitol, maltitol, isomaltitol, lactose, nigerose, kojibiose, xylitol, erythritol, dihydrochalcone, stevioside, α-glycosyl stevioside, acesulfame potassium, alitame, neotame, glycyrrhizin, thaumantin, sucralose, L-aspartyl-L-phenylalanine methyl ester, saccharine, maltodextrin, starch, potato starch, tapioca starch, dextran, soluble corn fiber, a resistant maltodextrin, a branched maltodextrin, inulin, polydextrose, a fructooligosaccharide, a galactooligosaccharide, a xylooligosaccharide, an arabinoxylooligosaccharide, a nigerooligosaccharide, a gentiooligosaccharide, hemicellulose, fructose oligomer syrup, an isomaltooligosaccharide, a filler, an excipient, a binder, or any combination thereof.

11. A method to reduce the glycemic index of a food or beverage comprising incorporating into the food or beverage the soluble α-glucan fiber composition of claim 1.

12. A method of inhibiting the elevation of blood-sugar level, lowering the lipid levels, treating constipation, or altering fatty acid production in a mammal comprising a step of administering the soluble α-glucan fiber composition of claim 1 to the mammal.

13. A cosmetic composition, a pharmaceutical composition, or a low cariogenicity composition comprising the soluble α-glucan fiber composition of claim 1.

14. Use of the soluble α-glucan fiber composition of claim 1 in a food composition suitable for consumption by animals, including humans.

15. A composition comprising 0.01 to 99 wt % (dry solids basis) of the soluble α-glucan fiber composition of claim 1 and: a synbiotic, a peptide, a peptide hydrolysate, a protein, a protein hydrolysate, a soy protein, a dairy protein, an amino acid, a polyol, a polyphenol, a vitamin, a mineral, an herbal, an herbal extract, a fatty acid, a polyunsaturated fatty acid (PUFAs), a phytosteroid, betaine, carotenoid, a digestive enzyme, a probiotic organism or any combination thereof.
